SEQUENCE LISTING (556 sequences)

SEQ ID NO: 1 - protein, 16 aa, synthetic construct
CACGTCTATACACCAC

SEQ ID NO: 2-16 - skipped (15 entries)

SEQ ID NO: 17 - protein, 16 aa, synthetic construct
TTTCTGAGGATTATCG

SEQ ID NO: 18 - protein, 16 aa, synthetic construct
TATCGTCAAGGCACTC

SEQ ID NO: 19 - protein, 16 aa, synthetic construct
CCTCATTGCACTGTAC

SEQ ID NO: 20 - protein, 16 aa, synthetic construct
TATCCAAACTGCCCTA

SEQ ID NO: 21-23 - skipped (3 entries)

SEQ ID NO: 24 - protein, 16 aa, synthetic construct
TGCCTACGCCACAAGC

SEQ ID NO: 25 - protein, 16 aa, synthetic construct
GCCTACGCCACAAGCT

SEQ ID NO: 26 - protein, 16 aa, synthetic construct
CCTACGCCACAAGCTC

SEQ ID NO: 27 - protein, 16 aa, synthetic construct
CTACGCCACAAGCTCC

SEQ ID NO: 28 - protein, 16 aa, synthetic construct
TACGCCACAAGCTCCA

SEQ ID NO: 29 - protein, 16 aa, synthetic construct
ACGCCACAAGCTCCAA

SEQ ID NO: 30 - protein, 16 aa, synthetic construct
CGCCACAAGCTCCAAC

SEQ ID NO: 31 - protein, 16 aa, synthetic construct
GCCACAAGCTCCAACT

SEQ ID NO: 32 - protein, 16 aa, synthetic construct
CCACAAGCTCCAACTA

SEQ ID NO: 33 - protein, 16 aa, synthetic construct
CACAAGCTCCAACTAC

SEQ ID NO: 34 - protein, 16 aa, synthetic construct
TTGCCTACGCCATCAG

SEQ ID NO: 35 - protein, 16 aa, synthetic construct
TGCCTACGCCATCAGC

SEQ ID NO: 36 - protein, 16 aa, synthetic construct
GCCTACGCCATCAGCT

SEQ ID NO: 37 - protein, 16 aa, synthetic construct
CCTACGCCATCAGCTC

SEQ ID NO: 38 - protein, 16 aa, synthetic construct
CTACGCCATCAGCTCC

SEQ ID NO: 39 - protein, 16 aa, synthetic construct
TACGCCATCAGCTCCA

SEQ ID NO: 40 - protein, 16 aa, synthetic construct
ACGCCATCAGCTCCAA

SEQ ID NO: 41 - protein, 16 aa, synthetic construct
CGCCATCAGCTCCAAC

SEQ ID NO: 42 - protein, 16 aa, synthetic construct
GCCATCAGCTCCAACT

SEQ ID NO: 43 - protein, 16 aa, synthetic construct
CCATCAGCTCCAACTA

SEQ ID NO: 44-64 - skipped (21 entries)

SEQ ID NO: 65 - protein, 15 aa, synthetic construct
ACGCCACAAGCTCCA

SEQ ID NO: 66 - protein, 14 aa, synthetic construct
CGCCACAAGCTCCA

SEQ ID NO: 67 - protein, 14 aa, synthetic construct
ACGCCACAAGCTCC

SEQ ID NO: 68 - protein, 15 aa, synthetic construct
CTACGCCATCAGCTC

SEQ ID NO: 69 - protein, 14 aa, synthetic construct
CTACGCCATCAGCT

SEQ ID NO: 70 - protein, 14 aa, synthetic construct
TACGCCATCAGCTC

SEQ ID NO: 71 - protein, 14 aa, synthetic construct
ACGCCATCAGCTCC

SEQ ID NO: 72 - protein, 16 aa, synthetic construct
CCTACGCCAGCAGCTC

SEQ ID NO: 73 - protein, 15 aa, synthetic construct
CTACGCCAGCAGCTC

SEQ ID NO: 74 - protein, 14 aa, synthetic construct
CTACGCCAGCAGCT

SEQ ID NO: 75 - protein, 14 aa, synthetic construct
TACGCCAGCAGCTC

SEQ ID NO: 76 - protein, 14 aa, synthetic construct
ACGCCAGCAGCTCC

SEQ ID NO: 77 - protein, 16 aa, synthetic construct
CCTACGCCAACAGCTC

SEQ ID NO: 78 - protein, 15 aa, synthetic construct
CTACGCCAACAGCTC

SEQ ID NO: 79 - protein, 14 aa, synthetic construct
CTACGCCAACAGCT

SEQ ID NO: 80 - protein, 14 aa, synthetic construct
TACGCCAACAGCTC

SEQ ID NO: 81 - protein, 14 aa, synthetic construct
ACGCCAACAGCTCC

SEQ ID NO: 82 - protein, 14 aa, synthetic construct
CGCCACAAGCTCCA

SEQ ID NO: 83-86 - skipped (4 entries)

SEQ ID NO: 87 - protein, 16 aa, synthetic construct
CTACGCCAGCAGCTCC

SEQ ID NO: 88-99 - skipped (12 entries)

SEQ ID NO: 100 - protein, 14 aa, synthetic construct
TCCTCTATTGTTGG

SEQ ID NO: 101 - protein, 14 aa, synthetic construct
ATCCTCTATTGTTG

SEQ ID NO: 102 - protein, 14 aa, synthetic construct
AATCCTCTATTGTT

SEQ ID NO: 103 - protein, 14 aa, synthetic construct
GAATCCTCTATTGT

SEQ ID NO: 104 - protein, 14 aa, synthetic construct
GGAATCCTCTATTG

SEQ ID NO: 105 - protein, 14 aa, synthetic construct
AGGAATCCTCTATT

SEQ ID NO: 106 - protein, 14 aa, synthetic construct
TAGGAATCCTCTAT

SEQ ID NO: 107 - protein, 14 aa, synthetic construct
GTAGGAATCCTCTA

SEQ ID NO: 108 - protein, 14 aa, synthetic construct
TGTAGGAATCCTCT

SEQ ID NO: 109 - protein, 14 aa, synthetic construct
CTGTAGGAATCCTC

SEQ ID NO: 110 - protein, 14 aa, synthetic construct
CCTGTAGGAATCCT

SEQ ID NO: 111 - protein, 14 aa, synthetic construct
TCCTGTAGGAATCC

SEQ ID NO: 112 - protein, 14 aa, synthetic construct
TTCCTGTAGGAATC

SEQ ID NO: 113 - protein, 14 aa, synthetic construct
CTTCCTGTAGGAAT

SEQ ID NO: 114 - protein, 14 aa, synthetic construct
GCTTCCTGTAGGAA

SEQ ID NO: 115 - protein, 14 aa, synthetic construct
TGCTTCCTGTAGGA

SEQ ID NO: 116 - protein, 14 aa, synthetic construct
TTGCTTCCTGTAGG

SEQ ID NO: 117 - protein, 14 aa, synthetic construct
CTTGCTTCCTGTAG

SEQ ID NO: 118 - protein, 14 aa, synthetic construct
ACTTGCTTCCTGTA

SEQ ID NO: 119 - protein, 14 aa, synthetic construct
TACTTGCTTCCTGT

SEQ ID NO: 120 - protein, 14 aa, synthetic construct
CTACTTGCTTCCTG

SEQ ID NO: 121 - protein, 14 aa, synthetic construct
ACTACTTGCTTCCT

SEQ ID NO: 122 - protein, 14 aa, synthetic construct
TACTACTTGCTTCC

SEQ ID NO: 123 - protein, 14 aa, synthetic construct
TTACTACTTGCTTC

SEQ ID NO: 124 - protein, 14 aa, synthetic construct
ATTACTACTTGCTT

SEQ ID NO: 125 - protein, 14 aa, synthetic construct
AATTACTACTTGCT

SEQ ID NO: 126 - protein, 14 aa, synthetic construct
CAATTACTACTTGC

SEQ ID NO: 127 - protein, 14 aa, synthetic construct
TCAATTACTACTTG

SEQ ID NO: 128 - protein, 14 aa, synthetic construct
ATCAATTACTACTT

SEQ ID NO: 129 - protein, 14 aa, synthetic construct
CATCAATTACTACT

SEQ ID NO: 130 - protein, 14 aa, synthetic construct
CCATCAATTACTAC

SEQ ID NO: 131 - protein, 14 aa, synthetic construct
TCCATCAATTACTA

SEQ ID NO: 132 - protein, 14 aa, synthetic construct
CTCCATCAATTACT

SEQ ID NO: 133 - protein, 14 aa, synthetic construct
TCTCCATCAATTAC

SEQ ID NO: 134 - protein, 14 aa, synthetic construct
TTCTCCATCAATTA

SEQ ID NO: 135 - protein, 14 aa, synthetic construct
TTTCTCCATCAATT

SEQ ID NO: 136 - protein, 14 aa, synthetic construct
GTTTCTCCATCAAT

SEQ ID NO: 137 - protein, 14 aa, synthetic construct
GGTTTCTCCATCAA

SEQ ID NO: 138 - protein, 14 aa, synthetic construct
AGGTTTCTCCATCA

SEQ ID NO: 139 - protein, 14 aa, synthetic construct
CAGGTTTCTCCATC

SEQ ID NO: 140 - protein, 14 aa, synthetic construct
ACAGGTTTCTCCAT

SEQ ID NO: 141 - protein, 14 aa, synthetic construct
GACAGGTTTCTCCA

SEQ ID NO: 142 - protein, 14 aa, synthetic construct
AGACAGGTTTCTCC

SEQ ID NO: 143 - protein, 14 aa, synthetic construct
GAGACAGGTTTCTC

SEQ ID NO: 144 - protein, 14 aa, synthetic construct
AGAGACAGGTTTCT

SEQ ID NO: 145 - protein, 14 aa, synthetic construct
AAGAGACAGGTTTC

SEQ ID NO: 146 - protein, 14 aa, synthetic construct
CAAGAGACAGGTTT

SEQ ID NO: 147 - protein, 14 aa, synthetic construct
CCAAGAGACAGGTT

SEQ ID NO: 148 - protein, 14 aa, synthetic construct
TCCAAGAGACAGGT

SEQ ID NO: 149 - protein, 14 aa, synthetic construct
ATCCAAGAGACAGG

SEQ ID NO: 150 - protein, 14 aa, synthetic construct
TATCCAAGAGACAG

SEQ ID NO: 151 - protein, 14 aa, synthetic construct
ATATCCAAGAGACA

SEQ ID NO: 152 - protein, 14 aa, synthetic construct
AATATCCAAGAGAC

SEQ ID NO: 153 - protein, 14 aa, synthetic construct
GAATATCCAAGAGA

SEQ ID NO: 154 - protein, 14 aa, synthetic construct
AGAATATCCAAGAG

SEQ ID NO: 155 - protein, 14 aa, synthetic construct
GAGAATATCCAAGA

SEQ ID NO: 156 - protein, 14 aa, synthetic construct
CGAGAATATCCAAG

SEQ ID NO: 157 - protein, 14 aa, synthetic construct
TCGAGAATATCCAA

SEQ ID NO: 158 - protein, 14 aa, synthetic construct
GTCGAGAATATCCA

SEQ ID NO: 159 - protein, 14 aa, synthetic construct
TGTCGAGAATATCC

SEQ ID NO: 160 - protein, 14 aa, synthetic construct
GTGTCGAGAATATC

SEQ ID NO: 161 - protein, 14 aa, synthetic construct
TGTGTCGAGAATAT

SEQ ID NO: 162 - protein, 14 aa, synthetic construct
CTGTGTCGAGAATA

SEQ ID NO: 163 - protein, 14 aa, synthetic construct
GCTGTGTCGAGAAT

SEQ ID NO: 164 - protein, 14 aa, synthetic construct
TGCTGTGTCGAGAA

SEQ ID NO: 165 - protein, 14 aa, synthetic construct
CTGCTGTGTCGAGA

SEQ ID NO: 166 - protein, 14 aa, synthetic construct
CCTGCTGTGTCGAG

SEQ ID NO: 167 - protein, 14 aa, synthetic construct
ACCTGCTGTGTCGA

SEQ ID NO: 168 - protein, 14 aa, synthetic construct
GACCTGCTGTGTCG

SEQ ID NO: 169 - protein, 14 aa, synthetic construct
TGACCTGCTGTGTC

SEQ ID NO: 170 - protein, 14 aa, synthetic construct
TTGACCTGCTGTGT

SEQ ID NO: 171 - protein, 14 aa, synthetic construct
CTTGACCTGCTGTG

SEQ ID NO: 172 - protein, 14 aa, synthetic construct
TCTTGACCTGCTGT

SEQ ID NO: 173 - protein, 14 aa, synthetic construct
CTCTTGACCTGCTG

SEQ ID NO: 174 - protein, 14 aa, synthetic construct
CCTCTTGACCTGCT

SEQ ID NO: 175 - protein, 14 aa, synthetic construct
TCCTCTTGACCTGC

SEQ ID NO: 176 - protein, 14 aa, synthetic construct
CTCCTCTTGACCTG

SEQ ID NO: 177 - protein, 14 aa, synthetic construct
ACTCCTCTTGACCT

SEQ ID NO: 178 - protein, 14 aa, synthetic construct
TACTCCTCTTGACC

SEQ ID NO: 179 - protein, 14 aa, synthetic construct
GTACTCCTCTTGAC

SEQ ID NO: 180 - protein, 14 aa, synthetic construct
TGTACTCCTCTTGA

SEQ ID NO: 181 - protein, 14 aa, synthetic construct
CTGTACTCCTCTTG

SEQ ID NO: 182 - protein, 14 aa, synthetic construct
ACTGTACTCCTCTT

SEQ ID NO: 183 - protein, 14 aa, synthetic construct
CACTGTACTCCTCT

SEQ ID NO: 184 - protein, 14 aa, synthetic construct
GCACTGTACTCCTC

SEQ ID NO: 185 - protein, 14 aa, synthetic construct
TGCACTGTACTCCT

SEQ ID NO: 186 - protein, 14 aa, synthetic construct
TTGCACTGTACTCC

SEQ ID NO: 187 - protein, 14 aa, synthetic construct
ATTGCACTGTACTC

SEQ ID NO: 188 - protein, 14 aa, synthetic construct
CATTGCACTGTACT

SEQ ID NO: 189 - protein, 14 aa, synthetic construct
TCATTGCACTGTAC

SEQ ID NO: 190 - protein, 14 aa, synthetic construct
CTCATTGCACTGTA

SEQ ID NO: 191 - protein, 14 aa, synthetic construct
CCTCATTGCACTGT

SEQ ID NO: 192 - protein, 14 aa, synthetic construct
CCCTCATTGCACTG

SEQ ID NO: 193 - protein, 14 aa, synthetic construct
TCCCTCATTGCACT

SEQ ID NO: 194 - protein, 14 aa, synthetic construct
GTCCCTCATTGCAC

SEQ ID NO: 195 - protein, 14 aa, synthetic construct
GGTCCCTCATTGCA

SEQ ID NO: 196 - protein, 14 aa, synthetic construct
TGGTCCCTCATTGC

SEQ ID NO: 197 - protein, 14 aa, synthetic construct
CTGGTCCCTCATTG

SEQ ID NO: 198 - protein, 14 aa, synthetic construct
ACTGGTCCCTCATT

SEQ ID NO: 199 - protein, 14 aa, synthetic construct
TACTGGTCCCTCAT

SEQ ID NO: 200 - protein, 14 aa, synthetic construct
GTACTGGTCCCTCA

SEQ ID NO: 201 - protein, 14 aa, synthetic construct
TGTACTGGTCCCTC

SEQ ID NO: 202 - protein, 14 aa, synthetic construct
ATGTACTGGTCCCT

SEQ ID NO: 203 - protein, 14 aa, synthetic construct
CATGTACTGGTCCC

SEQ ID NO: 204 - protein, 14 aa, synthetic construct
TCATGTACTGGTCC

SEQ ID NO: 205 - protein, 14 aa, synthetic construct
CTCATGTACTGGTC

SEQ ID NO: 206 - protein, 14 aa, synthetic construct
CCTCATGTACTGGT

SEQ ID NO: 207 - protein, 14 aa, synthetic construct
TCCTCATGTACTGG

SEQ ID NO: 208 - protein, 14 aa, synthetic construct
GTCCTCATGTACTG

SEQ ID NO: 209 - protein, 14 aa, synthetic construct
AGTCCTCATGTACT

SEQ ID NO: 210 - protein, 14 aa, synthetic construct
CAGTCCTCATGTAC

SEQ ID NO: 211 - protein, 14 aa, synthetic construct
CCAGTCCTCATGTA

SEQ ID NO: 212 - protein, 14 aa, synthetic construct
CCCAGTCCTCATGT

SEQ ID NO: 213 - protein, 14 aa, synthetic construct
CCCCAGTCCTCATG

SEQ ID NO: 214 - protein, 14 aa, synthetic construct
TCCCCAGTCCTCAT

SEQ ID NO: 215 - protein, 14 aa, synthetic construct
CTCCCCAGTCCTCA

SEQ ID NO: 216 - protein, 14 aa, synthetic construct
CCTCCCCAGTCCTC

SEQ ID NO: 217 - protein, 14 aa, synthetic construct
CCCTCCCCAGTCCT

SEQ ID NO: 218 - protein, 14 aa, synthetic construct
GCCCTCCCCAGTCC

SEQ ID NO: 219 - protein, 14 aa, synthetic construct
AGCCCTCCCCAGTC

SEQ ID NO: 220 - protein, 14 aa, synthetic construct
AAGCCCTCCCCAGT

SEQ ID NO: 221 - protein, 14 aa, synthetic construct
AAAGCCCTCCCCAG

SEQ ID NO: 222 - protein, 14 aa, synthetic construct
GAAAGCCCTCCCCA

SEQ ID NO: 223 - protein, 14 aa, synthetic construct
AGAAAGCCCTCCCC

SEQ ID NO: 224 - protein, 14 aa, synthetic construct
AAGAAAGCCCTCCC

SEQ ID NO: 225 - protein, 14 aa, synthetic construct
AAAGAAAGCCCTCC

SEQ ID NO: 226 - protein, 14 aa, synthetic construct
CAAAGAAAGCCCTC

SEQ ID NO: 227 - protein, 14 aa, synthetic construct
ACAAAGAAAGCCCT

SEQ ID NO: 228 - protein, 14 aa, synthetic construct
CACAAAGAAAGCCC

SEQ ID NO: 229 - protein, 14 aa, synthetic construct
ACACAAAGAAAGCC

SEQ ID NO: 230 - protein, 14 aa, synthetic construct
TACACAAAGAAAGC

SEQ ID NO: 231 - protein, 14 aa, synthetic construct
ATACACAAAGAAAG

SEQ ID NO: 232 - protein, 14 aa, synthetic construct
AATACACAAAGAAA

SEQ ID NO: 233 - protein, 14 aa, synthetic construct
AAATACACAAAGAA

SEQ ID NO: 234 - protein, 14 aa, synthetic construct
CAAATACACAAAGA

SEQ ID NO: 235 - protein, 14 aa, synthetic construct
GCAAATACACAAAG

SEQ ID NO: 236 - protein, 14 aa, synthetic construct
GGCAAATACACAAA

SEQ ID NO: 237 - protein, 14 aa, synthetic construct
TGGCAAATACACAA

SEQ ID NO: 238 - protein, 14 aa, synthetic construct
ATGGCAAATACACA

SEQ ID NO: 239 - protein, 14 aa, synthetic construct
TATGGCAAATACAC

SEQ ID NO: 240 - protein, 14 aa, synthetic construct
TTATGGCAAATACA

SEQ ID NO: 241 - protein, 14 aa, synthetic construct
TTTATGGCAAATAC

SEQ ID NO: 242 - protein, 14 aa, synthetic construct
ATTTATGGCAAATA

SEQ ID NO: 243 - protein, 14 aa, synthetic construct
TATTTATGGCAAAT

SEQ ID NO: 244 - protein, 14 aa, synthetic construct
TTATTTATGGCAAA

SEQ ID NO: 245 - protein, 14 aa, synthetic construct
ATTATTTATGGCAA

SEQ ID NO: 246 - protein, 14 aa, synthetic construct
TATTATTTATGGCA

SEQ ID NO: 247 - protein, 14 aa, synthetic construct
GTATTATTTATGGC

SEQ ID NO: 248 - protein, 14 aa, synthetic construct
AGTATTATTTATGG

SEQ ID NO: 249 - protein, 14 aa, synthetic construct
TAGTATTATTTATG

SEQ ID NO: 250 - protein, 14 aa, synthetic construct
TTAGTATTATTTAT

SEQ ID NO: 251 - protein, 14 aa, synthetic construct
TTTAGTATTATTTA

SEQ ID NO: 252 - protein, 14 aa, synthetic construct
ATTTAGTATTATTT

SEQ ID NO: 253 - protein, 14 aa, synthetic construct
GATTTAGTATTATT

SEQ ID NO: 254 - protein, 14 aa, synthetic construct
TGATTTAGTATTAT

SEQ ID NO: 255 - protein, 14 aa, synthetic construct
ATGATTTAGTATTA

SEQ ID NO: 256 - protein, 14 aa, synthetic construct
AATGATTTAGTATT

SEQ ID NO: 257 - protein, 14 aa, synthetic construct
AAATGATTTAGTAT

SEQ ID NO: 258 - protein, 14 aa, synthetic construct
CAAATGATTTAGTA

SEQ ID NO: 259 - protein, 14 aa, synthetic construct
TCAAATGATTTAGT

SEQ ID NO: 260 - protein, 14 aa, synthetic construct
TTCAAATGATTTAG

SEQ ID NO: 261 - protein, 14 aa, synthetic construct
CTTCAAATGATTTA

SEQ ID NO: 262 - protein, 14 aa, synthetic construct
TCTTCAAATGATTT

SEQ ID NO: 263 - protein, 14 aa, synthetic construct
ATCTTCAAATGATT

SEQ ID NO: 264 - protein, 14 aa, synthetic construct
TATCTTCAAATGAT

SEQ ID NO: 265 - protein, 14 aa, synthetic construct
ATATCTTCAAATGA

SEQ ID NO: 266 - protein, 14 aa, synthetic construct
AATATCTTCAAATG

SEQ ID NO: 267 - protein, 14 aa, synthetic construct
GAATATCTTCAAAT

SEQ ID NO: 268 - protein, 14 aa, synthetic construct
TGAATATCTTCAAA

SEQ ID NO: 269 - protein, 14 aa, synthetic construct
GTGAATATCTTCAA

SEQ ID NO: 270 - protein, 14 aa, synthetic construct
GGTGAATATCTTCA

SEQ ID NO: 271 - protein, 14 aa, synthetic construct
TGGTGAATATCTTC

SEQ ID NO: 272 - protein, 14 aa, synthetic construct
ATGGTGAATATCTT

SEQ ID NO: 273 - protein, 14 aa, synthetic construct
AATGGTGAATATCT

SEQ ID NO: 274 - protein, 14 aa, synthetic construct
TAATGGTGAATATC

SEQ ID NO: 275 - protein, 14 aa, synthetic construct
ATAATGGTGAATAT

SEQ ID NO: 276 - protein, 14 aa, synthetic construct
TATAATGGTGAATA

SEQ ID NO: 277 - protein, 14 aa, synthetic construct
CTATAATGGTGAAT

SEQ ID NO: 278 - protein, 14 aa, synthetic construct
TCTATAATGGTGAA

SEQ ID NO: 279 - protein, 14 aa, synthetic construct
CTCTATAATGGTGA

SEQ ID NO: 280 - protein, 14 aa, synthetic construct
TCTCTATAATGGTG

SEQ ID NO: 281 - protein, 14 aa, synthetic construct
TTCTCTATAATGGT

SEQ ID NO: 282 - protein, 14 aa, synthetic construct
GTTCTCTATAATGG

SEQ ID NO: 283 - protein, 14 aa, synthetic construct
TGTTCTCTATAATG

SEQ ID NO: 284 - protein, 14 aa, synthetic construct
TTGTTCTCTATAAT

SEQ ID NO: 285 - protein, 14 aa, synthetic construct
TTTGTTCTCTATAA

SEQ ID NO: 286 - protein, 14 aa, synthetic construct
ATTTGTTCTCTATA

SEQ ID NO: 287 - protein, 14 aa, synthetic construct
AATTTGTTCTCTAT

SEQ ID NO: 288 - protein, 14 aa, synthetic construct
TAATTTGTTCTCTA

SEQ ID NO: 289 - protein, 14 aa, synthetic construct
TTAATTTGTTCTCT

SEQ ID NO: 290 - protein, 14 aa, synthetic construct
TTTAATTTGTTCTC

SEQ ID NO: 291 - protein, 14 aa, synthetic construct
TTTTAATTTGTTCT

SEQ ID NO: 292 - protein, 14 aa, synthetic construct
CTTTTAATTTGTTC

SEQ ID NO: 293 - protein, 14 aa, synthetic construct
TCTTTTAATTTGTT

SEQ ID NO: 294 - protein, 14 aa, synthetic construct
CTCTTTTAATTTGT

SEQ ID NO: 295 - protein, 14 aa, synthetic construct
ACTCTTTTAATTTG

SEQ ID NO: 296 - protein, 14 aa, synthetic construct
AACTCTTTTAATTT

SEQ ID NO: 297 - protein, 14 aa, synthetic construct
TAACTCTTTTAATT

SEQ ID NO: 298 - protein, 14 aa, synthetic construct
TTAACTCTTTTAAT

SEQ ID NO: 299 - protein, 14 aa, synthetic construct
CTTAACTCTTTTAA

SEQ ID NO: 300 - protein, 14 aa, synthetic construct
CCTTAACTCTTTTA

SEQ ID NO: 301 - protein, 14 aa, synthetic construct
TCCTTAACTCTTTT

SEQ ID NO: 302 - protein, 14 aa, synthetic construct
GTCCTTAACTCTTT

SEQ ID NO: 303 - protein, 14 aa, synthetic construct
AGTCCTTAACTCTT

SEQ ID NO: 304 - protein, 14 aa, synthetic construct
GAGTCCTTAACTCT

SEQ ID NO: 305 - protein, 14 aa, synthetic construct
AGAGTCCTTAACTC

SEQ ID NO: 306 - protein, 14 aa, synthetic construct
CAGAGTCCTTAACT

SEQ ID NO: 307 - protein, 14 aa, synthetic construct
TCAGAGTCCTTAAC

SEQ ID NO: 308 - protein, 14 aa, synthetic construct
TTCAGAGTCCTTAA

SEQ ID NO: 309 - protein, 14 aa, synthetic construct
CTTCAGAGTCCTTA

SEQ ID NO: 310 - protein, 14 aa, synthetic construct
TCTTCAGAGTCCTT

SEQ ID NO: 311 - protein, 14 aa, synthetic construct
ATCTTCAGAGTCCT

SEQ ID NO: 312 - protein, 14 aa, synthetic construct
CATCTTCAGAGTCC

SEQ ID NO: 313 - protein, 14 aa, synthetic construct
ACATCTTCAGAGTC

SEQ ID NO: 314 - protein, 14 aa, synthetic construct
TACATCTTCAGAGT

SEQ ID NO: 315 - protein, 14 aa, synthetic construct
GTACATCTTCAGAG

SEQ ID NO: 316 - protein, 14 aa, synthetic construct
GGTACATCTTCAGA

SEQ ID NO: 317 - protein, 14 aa, synthetic construct
AGGTACATCTTCAG

SEQ ID NO: 318 - protein, 14 aa, synthetic construct
TAGGTACATCTTCA

SEQ ID NO: 319 - protein, 14 aa, synthetic construct
ATAGGTACATCTTC

SEQ ID NO: 320 - protein, 14 aa, synthetic construct
CATAGGTACATCTT

SEQ ID NO: 321 - protein, 14 aa, synthetic construct
CCATAGGTACATCT

SEQ ID NO: 322 - protein, 14 aa, synthetic construct
ACCATAGGTACATC

SEQ ID NO: 323 - protein, 14 aa, synthetic construct
GACCATAGGTACAT

SEQ ID NO: 324 - protein, 14 aa, synthetic construct
GGACCATAGGTACA

SEQ ID NO: 325 - protein, 14 aa, synthetic construct
AGGACCATAGGTAC

SEQ ID NO: 326 - protein, 14 aa, synthetic construct
TAGGACCATAGGTA

SEQ ID NO: 327 - protein, 14 aa, synthetic construct
CTAGGACCATAGGT

SEQ ID NO: 328 - protein, 14 aa, synthetic construct
ACTAGGACCATAGG

SEQ ID NO: 329 - protein, 14 aa, synthetic construct
TACTAGGACCATAG

SEQ ID NO: 330 - protein, 14 aa, synthetic construct
CTACTAGGACCATA

SEQ ID NO: 331 - protein, 14 aa, synthetic construct
CCTACTAGGACCAT

SEQ ID NO: 332 - protein, 14 aa, synthetic construct
TCCTACTAGGACCA

SEQ ID NO: 333 - protein, 14 aa, synthetic construct
TTCCTACTAGGACC

SEQ ID NO: 334 - protein, 14 aa, synthetic construct
TTTCCTACTAGGAC

SEQ ID NO: 335 - protein, 14 aa, synthetic construct
ATTTCCTACTAGGA

SEQ ID NO: 336 - protein, 14 aa, synthetic construct
TATTTCCTACTAGG

SEQ ID NO: 337 - protein, 14 aa, synthetic construct
TTATTTCCTACTAG

SEQ ID NO: 338 - protein, 14 aa, synthetic construct
TTTATTTCCTACTA

SEQ ID NO: 339 - protein, 14 aa, synthetic construct
ATTTATTTCCTACT

SEQ ID NO: 340 - protein, 14 aa, synthetic construct
CATTTATTTCCTAC

SEQ ID NO: 341 - protein, 14 aa, synthetic construct
ACATTTATTTCCTA

SEQ ID NO: 342 - protein, 14 aa, synthetic construct
CACATTTATTTCCT

SEQ ID NO: 343 - protein, 14 aa, synthetic construct
TCACATTTATTTCC

SEQ ID NO: 344 - protein, 14 aa, synthetic construct
ATCACATTTATTTC

SEQ ID NO: 345 - protein, 14 aa, synthetic construct
AATCACATTTATTT

SEQ ID NO: 346 - protein, 14 aa, synthetic construct
AAATCACATTTATT

SEQ ID NO: 347 - protein, 14 aa, synthetic construct
CAAATCACATTTAT

SEQ ID NO: 348 - protein, 14 aa, synthetic construct
GCAAATCACATTTA

SEQ ID NO: 349 - protein, 14 aa, synthetic construct
GGCAAATCACATTT

SEQ ID NO: 350 - protein, 14 aa, synthetic construct
AGGCAAATCACATT

SEQ ID NO: 351 - protein, 14 aa, synthetic construct
AAGGCAAATCACAT

SEQ ID NO: 352 - protein, 14 aa, synthetic construct
GAAGGCAAATCACA

SEQ ID NO: 353 - protein, 14 aa, synthetic construct
AGAAGGCAAATCAC

SEQ ID NO: 354 - protein, 14 aa, synthetic construct
TAGAAGGCAAATCA

SEQ ID NO: 355 - protein, 14 aa, synthetic construct
CTAGAAGGCAAATC

SEQ ID NO: 356 - protein, 14 aa, synthetic construct
TCTAGAAGGCAAAT

SEQ ID NO: 357 - protein, 14 aa, synthetic construct
TTCTAGAAGGCAAA

SEQ ID NO: 358 - protein, 14 aa, synthetic construct
GTTCTAGAAGGCAA

SEQ ID NO: 359 - protein, 14 aa, synthetic construct
TGTTCTAGAAGGCA

SEQ ID NO: 360 - protein, 14 aa, synthetic construct
CTGTTCTAGAAGGC

SEQ ID NO: 361 - protein, 14 aa, synthetic construct
ACTGTTCTAGAAGG

SEQ ID NO: 362 - protein, 14 aa, synthetic construct
TACTGTTCTAGAAG

SEQ ID NO: 363 - protein, 14 aa, synthetic construct
CTACTGTTCTAGAA

SEQ ID NO: 364 - protein, 14 aa, synthetic construct
TCTACTGTTCTAGA

SEQ ID NO: 365 - protein, 14 aa, synthetic construct
GTCTACTGTTCTAG

SEQ ID NO: 366 - protein, 14 aa, synthetic construct
TGTCTACTGTTCTA

SEQ ID NO: 367 - protein, 14 aa, synthetic construct
GTGTCTACTGTTCT

SEQ ID NO: 368 - protein, 14 aa, synthetic construct
TGTGTCTACTGTTC

SEQ ID NO: 369 - protein, 14 aa, synthetic construct
TTGTGTCTACTGTT

SEQ ID NO: 370 - protein, 14 aa, synthetic construct
TTTGTGTCTACTGT

SEQ ID NO: 371 - protein, 14 aa, synthetic construct
TTTTGTGTCTACTG

SEQ ID NO: 372 - protein, 14 aa, synthetic construct
GTTTTGTGTCTACT

SEQ ID NO: 373 - protein, 14 aa, synthetic construct
TGTTTTGTGTCTAC

SEQ ID NO: 374 - protein, 14 aa, synthetic construct
CTGTTTTGTGTCTA

SEQ ID NO: 375 - protein, 14 aa, synthetic construct
CCTGTTTTGTGTCT

SEQ ID NO: 376 - protein, 14 aa, synthetic construct
GCCTGTTTTGTGTC

SEQ ID NO: 377 - protein, 14 aa, synthetic construct
AGCCTGTTTTGTGT

SEQ ID NO: 378 - protein, 14 aa, synthetic construct
GAGCCTGTTTTGTG

SEQ ID NO: 379 - protein, 14 aa, synthetic construct
TGAGCCTGTTTTGT

SEQ ID NO: 380 - protein, 14 aa, synthetic construct
CTGAGCCTGTTTTG

SEQ ID NO: 381 - protein, 14 aa, synthetic construct
CCTGAGCCTGTTTT

SEQ ID NO: 382 - protein, 14 aa, synthetic construct
TCCTGAGCCTGTTT

SEQ ID NO: 383 - protein, 14 aa, synthetic construct
GTCCTGAGCCTGTT

SEQ ID NO: 384 - protein, 14 aa, synthetic construct
AGTCCTGAGCCTGT

SEQ ID NO: 385 - protein, 14 aa, synthetic construct
AAGTCCTGAGCCTG

SEQ ID NO: 386 - protein, 14 aa, synthetic construct
TAAGTCCTGAGCCT

SEQ ID NO: 387 - protein, 14 aa, synthetic construct
CTAAGTCCTGAGCC

SEQ ID NO: 388 - protein, 14 aa, synthetic construct
GCTAAGTCCTGAGC

SEQ ID NO: 389 - protein, 14 aa, synthetic construct
TGCTAAGTCCTGAG

SEQ ID NO: 390 - protein, 14 aa, synthetic construct
TTGCTAAGTCCTGA

SEQ ID NO: 391 - protein, 14 aa, synthetic construct
CTTGCTAAGTCCTG

SEQ ID NO: 392 - protein, 14 aa, synthetic construct
TCTTGCTAAGTCCT

SEQ ID NO: 393 - protein, 14 aa, synthetic construct
TTCTTGCTAAGTCC

SEQ ID NO: 394 - protein, 14 aa, synthetic construct
CTTCTTGCTAAGTC

SEQ ID NO: 395 - protein, 14 aa, synthetic construct
ACTTCTTGCTAAGT

SEQ ID NO: 396 - protein, 14 aa, synthetic construct
AACTTCTTGCTAAG

SEQ ID NO: 397 - protein, 14 aa, synthetic construct
TAACTTCTTGCTAA

SEQ ID NO: 398 - protein, 14 aa, synthetic construct
ATAACTTCTTGCTA

SEQ ID NO: 399 - protein, 14 aa, synthetic construct
CATAACTTCTTGCT

SEQ ID NO: 400 - protein, 14 aa, synthetic construct
CCATAACTTCTTGC

SEQ ID NO: 401 - protein, 14 aa, synthetic construct
TCCATAACTTCTTG

SEQ ID NO: 402 - protein, 14 aa, synthetic construct
TTCCATAACTTCTT

SEQ ID NO: 403 - protein, 14 aa, synthetic construct
ATTCCATAACTTCT

SEQ ID NO: 404 - protein, 14 aa, synthetic construct
AATTCCATAACTTC

SEQ ID NO: 405 - protein, 14 aa, synthetic construct
GAATTCCATAACTT

SEQ ID NO: 406 - protein, 14 aa, synthetic construct
GGAATTCCATAACT

SEQ ID NO: 407 - protein, 14 aa, synthetic construct
AGGAATTCCATAAC

SEQ ID NO: 408 - protein, 14 aa, synthetic construct
AAGGAATTCCATAA

SEQ ID NO: 409 - protein, 14 aa, synthetic construct
AAAGGAATTCCATA

SEQ ID NO: 410 - protein, 14 aa, synthetic construct
AAAAGGAATTCCAT

SEQ ID NO: 411 - protein, 14 aa, synthetic construct
TAAAAGGAATTCCA

SEQ ID NO: 412 - protein, 14 aa, synthetic construct
ATAAAAGGAATTCC

SEQ ID NO: 413 - protein, 14 aa, synthetic construct
AATAAAAGGAATTC

SEQ ID NO: 414 - protein, 14 aa, synthetic construct
CAATAAAAGGAATT

SEQ ID NO: 415 - protein, 14 aa, synthetic construct
TCAATAAAAGGAAT

SEQ ID NO: 416 - protein, 14 aa, synthetic construct
TTCAATAAAAGGAA

SEQ ID NO: 417 - protein, 14 aa, synthetic construct
TTTCAATAAAAGGA

SEQ ID NO: 418 - protein, 14 aa, synthetic construct
GTTTCAATAAAAGG

SEQ ID NO: 419 - protein, 14 aa, synthetic construct
TGTTTCAATAAAAG

SEQ ID NO: 420 - protein, 14 aa, synthetic construct
ATGTTTCAATAAAA

SEQ ID NO: 421 - protein, 14 aa, synthetic construct
GATGTTTCAATAAA

SEQ ID NO: 422 - protein, 14 aa, synthetic construct
TGATGTTTCAATAA

SEQ ID NO: 423 - protein, 14 aa, synthetic construct
CTGATGTTTCAATA

SEQ ID NO: 424 - protein, 14 aa, synthetic construct
GCTGATGTTTCAAT

SEQ ID NO: 425 - protein, 14 aa, synthetic construct
TGCTGATGTTTCAA

SEQ ID NO: 426 - protein, 14 aa, synthetic construct
TTGCTGATGTTTCA

SEQ ID NO: 427 - protein, 14 aa, synthetic construct
TTTGCTGATGTTTC

SEQ ID NO: 428 - protein, 14 aa, synthetic construct
CTTTGCTGATGTTT

SEQ ID NO: 429 - protein, 14 aa, synthetic construct
TCTTTGCTGATGTT

SEQ ID NO: 430 - protein, 14 aa, synthetic construct
GTCTTTGCTGATGT

SEQ ID NO: 431 - protein, 14 aa, synthetic construct
TGTCTTTGCTGATG

SEQ ID NO: 432 - protein, 14 aa, synthetic construct
TTGTCTTTGCTGAT

SEQ ID NO: 433 - protein, 14 aa, synthetic construct
CTTGTCTTTGCTGA

SEQ ID NO: 434 - protein, 14 aa, synthetic construct
TCTTGTCTTTGCTG

SEQ ID NO: 435 - protein, 14 aa, synthetic construct
GTCTTGTCTTTGCT

SEQ ID NO: 436 - protein, 14 aa, synthetic construct
TGTCTTGTCTTTGC

SEQ ID NO: 437 - protein, 14 aa, synthetic construct
CTGTCTTGTCTTTG

SEQ ID NO: 438 - protein, 14 aa, synthetic construct
TCTGTCTTGTCTTT

SEQ ID NO: 439 - protein, 14 aa, synthetic construct
CTCTGTCTTGTCTT

SEQ ID NO: 440 - protein, 14 aa, synthetic construct
TCTCTGTCTTGTCT

SEQ ID NO: 441 - protein, 14 aa, synthetic construct
CTCTCTGTCTTGTC

SEQ ID NO: 442 - protein, 14 aa, synthetic construct
ACTCTCTGTCTTGT

SEQ ID NO: 443 - protein, 14 aa, synthetic construct
CACTCTCTGTCTTG

SEQ ID NO: 444 - protein, 14 aa, synthetic construct
CCACTCTCTGTCTT

SEQ ID NO: 445 - protein, 14 aa, synthetic construct
TCCACTCTCTGTCT

SEQ ID NO: 446 - protein, 14 aa, synthetic construct
CTCCACTCTCTGTC

SEQ ID NO: 447 - protein, 14 aa, synthetic construct
CCTCCACTCTCTGT

SEQ ID NO: 448 - protein, 14 aa, synthetic construct
TCCTCCACTCTCTG

SEQ ID NO: 449 - protein, 14 aa, synthetic construct
ATCCTCCACTCTCT

SEQ ID NO: 450 - protein, 14 aa, synthetic construct
CATCCTCCACTCTC

SEQ ID NO: 451 - protein, 14 aa, synthetic construct
GCATCCTCCACTCT

SEQ ID NO: 452 - protein, 14 aa, synthetic construct
AGCATCCTCCACTC

SEQ ID NO: 453 - protein, 14 aa, synthetic construct
AAGCATCCTCCACT

SEQ ID NO: 454 - protein, 14 aa, synthetic construct
AAAGCATCCTCCAC

SEQ ID NO: 455 - protein, 14 aa, synthetic construct
AAAAGCATCCTCCA

SEQ ID NO: 456 - protein, 14 aa, synthetic construct
AAAAAGCATCCTCC

SEQ ID NO: 457 - protein, 14 aa, synthetic construct
TAAAAAGCATCCTC

SEQ ID NO: 458 - protein, 14 aa, synthetic construct
ATAAAAAGCATCCT

SEQ ID NO: 459 - protein, 14 aa, synthetic construct
TATAAAAAGCATCC

SEQ ID NO: 460 - protein, 14 aa, synthetic construct
GTATAAAAAGCATC

SEQ ID NO: 461 - protein, 14 aa, synthetic construct
TGTATAAAAAGCAT

SEQ ID NO: 462 - protein, 14 aa, synthetic construct
ATGTATAAAAAGCA

SEQ ID NO: 463 - protein, 14 aa, synthetic construct
AATGTATAAAAAGC

SEQ ID NO: 464 - protein, 14 aa, synthetic construct
CAATGTATAAAAAG

SEQ ID NO: 465 - protein, 14 aa, synthetic construct
CCAATGTATAAAAA

SEQ ID NO: 466 - protein, 14 aa, synthetic construct
ACCAATGTATAAAA

SEQ ID NO: 467 - protein, 14 aa, synthetic construct
CACCAATGTATAAA

SEQ ID NO: 468 - protein, 14 aa, synthetic construct
TCACCAATGTATAA

SEQ ID NO: 469 - protein, 14 aa, synthetic construct
CTCACCAATGTATA

SEQ ID NO: 470 - protein, 14 aa, synthetic construct
TCTCACCAATGTAT

SEQ ID NO: 471 - protein, 14 aa, synthetic construct
CTCTCACCAATGTA

SEQ ID NO: 472 - protein, 14 aa, synthetic construct
TCTCTCACCAATGT

SEQ ID NO: 473 - protein, 14 aa, synthetic construct
CTCTCTCACCAATG

SEQ ID NO: 474 - protein, 14 aa, synthetic construct
TCTCTCTCACCAAT

SEQ ID NO: 475 - protein, 14 aa, synthetic construct
ATCTCTCTCACCAA

SEQ ID NO: 476 - protein, 14 aa, synthetic construct
GATCTCTCTCACCA

SEQ ID NO: 477 - protein, 14 aa, synthetic construct
GGATCTCTCTCACC

SEQ ID NO: 478 - protein, 14 aa, synthetic construct
CGGATCTCTCTCAC

SEQ ID NO: 479 - protein, 14 aa, synthetic construct
TCGGATCTCTCTCA

SEQ ID NO: 480 - protein, 14 aa, synthetic construct
GTCGGATCTCTCTC

SEQ ID NO: 481 - protein, 14 aa, synthetic construct
TGTCGGATCTCTCT

SEQ ID NO: 482 - protein, 14 aa, synthetic construct
TTGTCGGATCTCTC

SEQ ID NO: 483 - protein, 14 aa, synthetic construct
ATTGTCGGATCTCT

SEQ ID NO: 484 - protein, 14 aa, synthetic construct
TATTGTCGGATCTC

SEQ ID NO: 485 - protein, 14 aa, synthetic construct
GTATTGTCGGATCT

SEQ ID NO: 486 - protein, 14 aa, synthetic construct
TGTATTGTCGGATC

SEQ ID NO: 487 - protein, 14 aa, synthetic construct
CTGTATTGTCGGAT

SEQ ID NO: 488 - protein, 14 aa, synthetic construct
TCTGTATTGTCGGA

SEQ ID NO: 489 - protein, 14 aa, synthetic construct
ATCTGTATTGTCGG

SEQ ID NO: 490 - protein, 14 aa, synthetic construct
AATCTGTATTGTCG

SEQ ID NO: 491 - protein, 14 aa, synthetic construct
CAATCTGTATTGTC

SEQ ID NO: 492 - protein, 14 aa, synthetic construct
TCAATCTGTATTGT

SEQ ID NO: 493 - protein, 14 aa, synthetic construct
TTCAATCTGTATTG

SEQ ID NO: 494 - protein, 14 aa, synthetic construct
TTTCAATCTGTATT

SEQ ID NO: 495 - protein, 14 aa, synthetic construct
TTTTCAATCTGTAT

SEQ ID NO: 496 - protein, 14 aa, synthetic construct
TTTTTCAATCTGTA

SEQ ID NO: 497 - protein, 14 aa, synthetic construct
TTTTTTCAATCTGT

SEQ ID NO: 498 - protein, 14 aa, synthetic construct
TTTTTTTCAATCTG

SEQ ID NO: 499 - protein, 14 aa, synthetic construct
ATTTTTTTCAATCT

SEQ ID NO: 500 - protein, 14 aa, synthetic construct
GATTTTTTTCAATC

SEQ ID NO: 501 - protein, 14 aa, synthetic construct
TGATTTTTTTCAAT

SEQ ID NO: 502 - protein, 14 aa, synthetic construct
CTGATTTTTTTCAA

SEQ ID NO: 503 - protein, 14 aa, synthetic construct
GCTGATTTTTTTCA

SEQ ID NO: 504 - protein, 14 aa, synthetic construct
TGCTGATTTTTTTC

SEQ ID NO: 505 - protein, 14 aa, synthetic construct
TTGCTGATTTTTTT

SEQ ID NO: 506 - protein, 14 aa, synthetic construct
TTTGCTGATTTTTT

SEQ ID NO: 507 - protein, 14 aa, synthetic construct
CTTTGCTGATTTTT

SEQ ID NO: 508 - protein, 14 aa, synthetic construct
TCTTTGCTGATTTT

SEQ ID NO: 509 - protein, 14 aa, synthetic construct
TTCTTTGCTGATTT

SEQ ID NO: 510 - protein, 14 aa, synthetic construct
CTTCTTTGCTGATT

SEQ ID NO: 511 - protein, 14 aa, synthetic construct
TCTTCTTTGCTGAT

SEQ ID NO: 512 - protein, 14 aa, synthetic construct
TTCTTCTTTGCTGA

SEQ ID NO: 513 - protein, 14 aa, synthetic construct
TTTCTTCTTTGCTG

SEQ ID NO: 514 - protein, 14 aa, synthetic construct
TTTTCTTCTTTGCT

SEQ ID NO: 515 - protein, 14 aa, synthetic construct
CTTTTCTTCTTTGC

SEQ ID NO: 516 - protein, 14 aa, synthetic construct
TCTTTTCTTCTTTG

SEQ ID NO: 517 - protein, 14 aa, synthetic construct
GTCTTTTCTTCTTT

SEQ ID NO: 518 - protein, 14 aa, synthetic construct
AGTCTTTTCTTCTT

SEQ ID NO: 519 - protein, 14 aa, synthetic construct
GAGTCTTTTCTTCT

SEQ ID NO: 520 - protein, 14 aa, synthetic construct
GGAGTCTTTTCTTC

SEQ ID NO: 521 - protein, 14 aa, synthetic construct
AGGAGTCTTTTCTT

SEQ ID NO: 522 - protein, 14 aa, synthetic construct
CAGGAGTCTTTTCT

SEQ ID NO: 523 - protein, 14 aa, synthetic construct
CCAGGAGTCTTTTC

SEQ ID NO: 524 - protein, 14 aa, synthetic construct
GCCAGGAGTCTTTT

SEQ ID NO: 525 - protein, 14 aa, synthetic construct
AGCCAGGAGTCTTT

SEQ ID NO: 526 - protein, 14 aa, synthetic construct
CAGCCAGGAGTCTT

SEQ ID NO: 527 - protein, 14 aa, synthetic construct
ACAGCCAGGAGTCT

SEQ ID NO: 528 - protein, 14 aa, synthetic construct
CACAGCCAGGAGTC

SEQ ID NO: 529 - protein, 14 aa, synthetic construct
ACACAGCCAGGAGT

SEQ ID NO: 530 - protein, 14 aa, synthetic construct
CACACAGCCAGGAG

SEQ ID NO: 531 - protein, 14 aa, synthetic construct
TCACACAGCCAGGA

SEQ ID NO: 532 - protein, 14 aa, synthetic construct
TTCACACAGCCAGG

SEQ ID NO: 533 - protein, 14 aa, synthetic construct
TTTCACACAGCCAG

SEQ ID NO: 534 - protein, 14 aa, synthetic construct
TTTTCACACAGCCA

SEQ ID NO: 535 - protein, 14 aa, synthetic construct
ATTTTCACACAGCC

SEQ ID NO: 536 - protein, 14 aa, synthetic construct
AATTTTCACACAGC

SEQ ID NO: 537 - protein, 14 aa, synthetic construct
TAATTTTCACACAG

SEQ ID NO: 538 - protein, 14 aa, synthetic construct
TTAATTTTCACACA

SEQ ID NO: 539 - protein, 14 aa, synthetic construct
TTTAATTTTCACAC

SEQ ID NO: 540 - protein, 14 aa, synthetic construct
TTTTAATTTTCACA

SEQ ID NO: 541 - protein, 14 aa, synthetic construct
TTTTTAATTTTCAC

SEQ ID NO: 542 - protein, 14 aa, synthetic construct
TTTTTTAATTTTCA

SEQ ID NO: 543 - protein, 14 aa, synthetic construct
ATTTTTTAATTTTC

SEQ ID NO: 544 - protein, 14 aa, synthetic construct
CATTTTTTAATTTT

SEQ ID NO: 545 - protein, 14 aa, synthetic construct
GCATTTTTTAATTT

SEQ ID NO: 546 - protein, 14 aa, synthetic construct
TGCATTTTTTAATT

SEQ ID NO: 547 - protein, 14 aa, synthetic construct
ATGCATTTTTTAAT

SEQ ID NO: 548 - protein, 14 aa, synthetic construct
AATGCATTTTTTAA

SEQ ID NO: 549 - protein, 14 aa, synthetic construct
TAATGCATTTTTTA

SEQ ID NO: 550 - protein, 14 aa, synthetic construct
ATAATGCATTTTTT

SEQ ID NO: 551 - protein, 14 aa, synthetic construct
TATAATGCATTTTT

SEQ ID NO: 552 - protein, 14 aa, synthetic construct
TTATAATGCATTTT

SEQ ID NO: 553 - protein, 14 aa, synthetic construct
ATTATAATGCATTT

SEQ ID NO: 554 - protein, 14 aa, synthetic construct
CATTATAATGCATT

SEQ ID NO: 555 - protein, 14 aa, synthetic construct
ACATTATAATGCAT

SEQ ID NO: 556 - protein, 14 aa, synthetic construct
TACATTATAATGCA

(19) United States
(12) Patent Application Publication
(10) Pub. No.: US 2024/0392293 A1
(43) Pub. Date: Nov. 28, 2024

(54) COMPOSITIONS AND METHODS FOR MODULATING KRAS EXPRESSION

(71) Applicant: Molecular Axiom, LLC, San Diego, CA (US)

(72) Inventors: Lishan CHEN, San Diego, CA (US); Bohan JIN, San Diego, CA (US)

(21) Appl. No.: 18/688,568

(22) PCT Filed: Sep. 1, 2022
(86) PCT No.: PCT/US2022/042393

§ 371 Date: Mar. 1, 2024

Related U.S. Application Data
(60) Provisional application No. 63/315,669, filed on Mar. 2, 2022.
(60) Provisional application No. 63/240,226, filed on Sep. 2, 2021.

(51) Int. Cl. C12N 15/113; A61P 35/00

(52) U.S. Cl. CPC C12N 15/1135; A61P 35/00; C12N 2310/11; C12N 2310/313; C12N 2310/315; C12N 2310/321; C12N 2310/322; C12N 2310/3231; C12N 2310/351; C12N 2320/31

(57) ABSTRACT

Described herein are compositions for modulating gene expressions. Also described herein are methods for using the compositions described herein for modulating gene expressions.

### CROSS-REFERENCE

This application claims the benefit of U.S. Provisional Application Ser. No. 63/240,226 filed on Sep. 2, 2021, and U.S. Provisional Application Ser. No. 63/315,669 filed on Mar. 2, 2022, the entirety of which is hereby incorporated by reference herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BACKGROUND

Certain diseases or conditions are caused by genetic mutations or deregulated signaling pathways due to over- or under-expression of one or more genes affecting the signaling pathway. To treat such diseases or conditions, one of the most sought after treatment option involves direct editing of the genetic mutations, or transcriptional/translational regulation using gene silencing tools or methods. RNA-induced gene silencing controls RNA expression of target genes in various aspects including transcription inactivation, mRNA degradation, transcriptional attenuation. Therefore, there remains a need for compositions and methods for effective editing gene expression at RNA levels.

### SUMMARY

Described herein, in some aspects, is composition comprising an antisense oligonucleotide capable of binding to KRAS mRNA. In some embodiments, the KRAS mRNA is a mutated KRAS mRNA. In some embodiments, the antisense oligonucleotide comprises a sequence that is at least 80%, 85%, or 90% identical to one of the following sequences: SEQ ID NOs: 100-556. In some embodiments, the antisense oligonucleotide comprises a sequence that is at least 80%, 85%, or 90% identical to any one of the following sequences: SEQ ID NOs: 24-43, 65-82, or 87. In some embodiments, the antisense oligonucleotide comprises a sequence that is at least 80%, 85%, or 90% identical to any one of the following sequences: SEQ ID NO: 129, SEQ ID NO: 213, SEQ ID NO: 214, SEQ ID NO: 215, SEQ ID NO: 216, SEQ ID NO: 217, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 392, SEQ ID NO: 393, SEQ ID NO: 394, SEQ ID NO: 399, SEQ ID NO: 400, SEQ ID NO: 401, SEQ ID NO: 402, SEQ ID NO: 427, SEQ ID NO: 428, SEQ ID NO: 429, SEQ ID NO: 430, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 494, SEQ ID NO: 495, SEQ ID NO: 496, SEQ ID NO: 497, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 18, SEQ ID NO: 19, or SEQ ID NO: 20. In some embodiments, the antisense oligonucleotide comprises a nucleic acid sequence that is at least 80%, 85%, or 90% identical to any one of SEQ ID NO: 18-20, SEQ ID NOs: 24-43, SEQ ID NO: 65-82, or SEQ ID NO: 87. In some embodiments, the antisense oligonucleotide comprises 12-30 nucleotides in length. In some embodiments, the antisense oligonucleotide comprises a gap segment and a wing segment. In some embodiments, the antisense oligonucleotide comprises a 5′-wing segment and a 3′-wing segment. In some embodiments, the each of the 5′-wing segment and 3′-wing segment is three linked nucleotides. In some embodiments, the antisense oligonucleotide comprises at least one 2′-modified nucleoside, at least one modified internucleotide linkage, or at least one inverted abasic moiety. In some embodiments, the at least one 2′ modified nucleotide: comprises 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleotide, locked nucleic acid (LNA), constrained ethyl (cEt) sugar, ethylene nucleic acid (ENA), or a combination thereof. In some embodiments, the at least one modified internucleotide linkage comprises a phosphorothioate linkage or a phosphorodithioate linkage. In some embodiments, the antisense oligonucleotide comprises a phosphorodiamidate morpholino oligomer (PMO), locked nucleic acid (LNA), a thiomorpholino, constrained ethyl (cEt) sugar, or a combination thereof. In some embodiments, the antisense oligonucleotide is conjugated with a peptide, antibody, lipid, carbohydrates, aptamer or a polymer. In some embodiments, the antisense oligonucleotide is conjugated with a peptide, antibody, lipid, carbohydrates, aptamer or a polymer via a linker. In some embodiments, the composition comprises a combination of an antisense oligonucleotide specifically binds to the KRAS mRNA and an antisense oligonucleotide specifically binds to the mutated KRAS mRNA. In some embodiments, the composition comprises an antisense oligonucleotide capable of binding to both KRAS mRNA and mutated KRAS mRNA. In some embodiments, the composition further comprises an excipient. In some embodiments, the composition is formulated for parenteral or inhalation administration. In some embodiments, the mutated KRAS mRNA encodes a mutated KRAS protein comprising a G12C mutation, a G12V mutation, a G12A mutation, or a G12D mutation. In some embodiments, the antisense oligonucleotide comprises a nucleic acid sequence that is at least 80%, 85%, or 90% identical to any one of SEQ ID NOs: 19, 27, 28, 37, 44, or 65-81. In some embodiments, the antisense oligonucleotide comprises a nucleic acid sequence that is at least 80%, 85%, or 90% identical to any one of SEQ ID NOs: 19, 28, 44, 67, 72-77, or 79-81. In some embodiments, the antisense oligonucleotide comprises a nucleic acid sequence that is at least 80%, 85%, or 90% identical to SEQ ID NO: 19. In some embodiments, the antisense oligonucleotide comprises a nucleic acid sequence that is at least 80%, 85%, or 90% identical to SEQ ID NO: 28. In some embodiments, the antisense oligonucleotide comprises a nucleic acid sequence that is at least 80%, 85%, or 90% identical to SEQ ID NO: 44. In some embodiments, the antisense oligonucleotide comprises a nucleic acid sequence that is at least 80%, 85%, or 90% identical to SEQ ID NO: 67. In some embodiments, the antisense oligonucleotide comprises a nucleic acid sequence that is at least 80%, 85%, or 90% identical to SEQ ID NO: 72. In some embodiments, the antisense oligonucleotide comprises a nucleic acid sequence that is at least 80%, 85%, or 90% identical to SEQ ID NO: 73. In some embodiments, the antisense oligonucleotide comprises a nucleic acid sequence that is at least 80%, 85%, or 90% identical to SEQ ID NO: 74. In some embodiments, the antisense oligonucleotide comprises a nucleic acid sequence that is at least 80%, 85%, or 90% identical to SEQ ID NO: 75. In some embodiments, the antisense oligonucleotide comprises a nucleic acid sequence that is at least 80%, 85%, or 90% identical to SEQ ID NO: 76. In some embodiments, the antisense oligonucleotide comprises a nucleic acid sequence that is at least 80%, 85%, or 90% identical to SEQ ID NO: 77. In some embodiments, the antisense oligonucleotide comprises a nucleic acid sequence that is at least 80%, 85%, or 90% identical to SEQ ID NO: 79. In some embodiments, the antisense oligonucleotide comprises a nucleic acid sequence that is at least 80%, 85%, or 90% identical to SEQ ID NO: 80. In some embodiments, the antisense oligonucleotide comprises a nucleic acid sequence that is at least 80%, 85%, or 90% identical to SEQ ID NO: 81. In some embodiments, the antisense oligonucleotide comprises a nucleic acid sequence that is any one of SEQ ID NOs: 19, 27, 28, 37, 44, or 65-81. In some embodiments, the antisense oligonucleotide comprises a nucleic acid sequence that is at any one of SEQ ID NOs: 19, 28, 44, 67, 72-77, or 79-81. In some embodiments, the antisense oligonucleotide comprises a nucleic acid sequence of SEQ ID NO: 19. In some embodiments, the antisense oligonucleotide comprises a nucleic acid sequence of SEQ ID NO: 28. In some embodiments, the antisense oligonucleotide comprises a nucleic acid sequence of SEQ ID NO: 44. In some embodiments, the antisense oligonucleotide comprises a nucleic acid sequence of SEQ ID NO: 67. In some embodiments, the antisense oligonucleotide comprises a nucleic acid sequence of SEQ ID NO: 72. In some embodiments, the antisense oligonucleotide comprises a nucleic acid sequence of SEQ ID NO: 73. In some embodiments, the antisense oligonucleotide comprises a nucleic acid sequence of SEQ ID NO: 74. In some embodiments, the antisense oligonucleotide comprises a nucleic acid sequence of SEQ ID NO: 75. In some embodiments, the antisense oligonucleotide comprises a nucleic acid sequence of SEQ ID NO: 76. In some embodiments, the antisense oligonucleotide comprises a nucleic acid sequence of SEQ ID NO: 77. In some embodiments, the antisense oligonucleotide comprises a nucleic acid sequence of SEQ ID NO: 79. In some embodiments, the antisense oligonucleotide comprises a nucleic acid sequence of SEQ ID NO: 80. In some embodiments, the antisense oligonucleotide comprises a nucleic acid sequence of SEQ ID NO: 81.

Described herein, in some aspects, is a method of modulating KRAS-mediated signaling pathway in a cancer cell, comprising: treating the cancer cell with a composition comprising antisense oligonucleotide capable of binding to KRAS mRNA or mutated KRAS mRNA, thereby reducing expression of KRAS mRNA or mutated KRAS mRNA in the cancer cell. In some embodiments, the cancer cell is a lung cancer cell, a pancreatic cancer cell, or a colon cancer cell. In some embodiments, the antisense oligonucleotide comprises a sequence having at least 80%, 85%, or 90% similarity to one of the following sequences: SEQ ID NOs: 100-556. In some embodiments, the antisense oligonucleotide comprises a sequence having at least 80%, 85%, or 90% similarity to one of the following sequences: SEQ ID NOs: 24-43, 65-82, or 87. In some embodiments, the antisense oligonucleotide comprises a sequence having at least 80%, 85%, or 90% similarity to one of the following sequences: SEQ ID NO: 129, SEQ ID NO: 213, SEQ ID NO: 214, SEQ ID NO: 215, SEQ ID NO: 216, SEQ ID NO: 217, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 392, SEQ ID NO: 393, SEQ ID NO: 394, SEQ ID NO: 399, SEQ ID NO: 400, SEQ ID NO: 401, SEQ ID NO: 402, SEQ ID NO: 427, SEQ ID NO: 428, SEQ ID NO: 429, SEQ ID NO: 430, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 494, SEQ ID NO: 495, SEQ ID NO: 496, SEQ ID NO: 497, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 18, SEQ ID NO: 19, or SEQ ID NO: 20. In some embodiments, the composition comprises a combination of an antisense oligonucleotide specifically binds to the KRAS mRNA and an antisense oligonucleotide specifically binds to the mutated KRAS mRNA. In some embodiments, the composition comprises an antisense oligonucleotide capable of binding to both KRAS mRNA and mutated KRAS mRNA. In some embodiments, the antisense oligonucleotide comprises at least one 2′-modified nucleoside, at least one modified internucleotide linkage, or at least one inverted abasic moiety. In some embodiments, the at least one 2′ modified nucleotide: comprises 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleotide; comprises locked nucleic acid (LNA), locked nucleic acid (LNA), constrained ethyl (cEt) sugar or ethylene nucleic acid (ENA), a thiomorpholino, or a combination thereof. In some embodiments, the expression of KRAS protein, mutated KRAS protein, KRAS mRNA, or mutated KRAS mRNA is decreased by at least 30%, at least 40%, at least 50% after the treatment. In some embodiments, the antisense oligonucleotide comprises a nucleic acid sequence that is at least 80%, 85%, or 90% identical to any one of SEQ ID NOs: 19, 27, 28, 37, 44, or 65-81. In some embodiments, the antisense oligonucleotide comprises a nucleic acid sequence that is at least 80%, 85%, or 90% identical to any one of SEQ ID NOs: 19, 28, 44, 67, 72-77, or 79-81. In some embodiments, the antisense oligonucleotide comprises a nucleic acid sequence that is at least 80%, 85%, or 90% identical to SEQ ID NO: 19. In some embodiments, the antisense oligonucleotide comprises a nucleic acid sequence that is at least 80%, 85%, or 90% identical to SEQ ID NO: 28. In some embodiments, the antisense oligonucleotide comprises a nucleic acid sequence that is at least 80%, 85%, or 90% identical to SEQ ID NO: 44. In some embodiments, the antisense oligonucleotide comprises a nucleic acid sequence that is at least 80%, 85%, or 90% identical to SEQ ID NO: 67. In some embodiments, the antisense oligonucleotide comprises a nucleic acid sequence that is at least 80%, 85%, or 90% identical to SEQ ID NO: 72. In some embodiments, the antisense oligonucleotide comprises a nucleic acid sequence that is at least 80%, 85%, or 90% identical to SEQ ID NO: 73. In some embodiments, the antisense oligonucleotide comprises a nucleic acid sequence that is at least 80%, 85%, or 90% identical to SEQ ID NO: 74. In some embodiments, the antisense oligonucleotide comprises a nucleic acid sequence that is at least 80%, 85%, or 90% identical to SEQ ID NO: 75. In some embodiments, the antisense oligonucleotide comprises a nucleic acid sequence that is at least 80%, 85%, or 90% identical to SEQ ID NO: 76. In some embodiments, the antisense oligonucleotide comprises a nucleic acid sequence that is at least 80%, 85%, or 90% identical to SEQ ID NO: 77. In some embodiments, the antisense oligonucleotide comprises a nucleic acid sequence that is at least 80%, 85%, or 90% identical to SEQ ID NO: 79. In some embodiments, the antisense oligonucleotide comprises a nucleic acid sequence that is at least 80%, 85%, or 90% identical to SEQ ID NO: 80. In some embodiments, the antisense oligonucleotide comprises a nucleic acid sequence that is at least 80%, 85%, or 90% identical to SEQ ID NO: 81. In some embodiments, the antisense oligonucleotide comprises a nucleic acid sequence that is any one of SEQ ID NOs: 19, 27, 28, 37, 44, or 65-81. In some embodiments, the antisense oligonucleotide comprises a nucleic acid sequence that is at any one of SEQ ID NOs: 19, 28, 44, 67, 72-77, or 79-81. In some embodiments, the antisense oligonucleotide comprises a nucleic acid sequence of SEQ ID NO: 19. In some embodiments, the antisense oligonucleotide comprises a nucleic acid sequence of SEQ ID NO: 28. In some embodiments, the antisense oligonucleotide comprises a nucleic acid sequence of SEQ ID NO: 44. In some embodiments, the antisense oligonucleotide comprises a nucleic acid sequence of SEQ ID NO: 67. In some embodiments, the antisense oligonucleotide comprises a nucleic acid sequence of SEQ ID NO: 72. In some embodiments, the antisense oligonucleotide comprises a nucleic acid sequence of SEQ ID NO: 73. In some embodiments, the antisense oligonucleotide comprises a nucleic acid sequence of SEQ ID NO: 74. In some embodiments, the antisense oligonucleotide comprises a nucleic acid sequence of SEQ ID NO: 75. In some embodiments, the antisense oligonucleotide comprises a nucleic acid sequence of SEQ ID NO: 76. In some embodiments, the antisense oligonucleotide comprises a nucleic acid sequence of SEQ ID NO: 77. In some embodiments, the antisense oligonucleotide comprises a nucleic acid sequence of SEQ ID NO: 79. In some embodiments, the antisense oligonucleotide comprises a nucleic acid sequence of SEQ ID NO: 80. In some embodiments, the antisense oligonucleotide comprises a nucleic acid sequence of SEQ ID NO: 81.

Described herein, in some aspects, is a method of treating a cancer in a subject in need thereof, the method comprising: administering the subject a composition comprising an antisense oligonucleotide described herein, thereby treating the cancer in the subject. In some embodiments, the cancer is associated with an abnormality of KRAS-mediated signaling pathway. In some embodiments, the caner is a lung cancer, a pancreatic cancer, or a colon cancer. In some embodiments, the composition is administered to the subject in a dose and schedule sufficient to increase survival rate of the subject at least 5%. In some embodiments, the composition is administered to the subject in a dose and schedule sufficient to inhibit growth of the tumor. In some embodiments, the cancer is associated with KRAS or mutated KRAS. In some embodiments, the mutated KRAS mRNA encodes a mutated KRAS protein comprising a G12C mutation, a G12V mutation, a G12A mutation, or a G12D mutation.

### BRIEF DESCRIPTION OF THE DRAWINGS

This patent application contains at least one drawing executed in color. Copies of this patent or patent application with color drawing(s) will be provided by the Office upon request and payment of the necessary fee.

FIG. 1 illustrates knockdown of KRAS mRNA encoding a mutated KRAS protein comprising the G12C mutation mediated by the oligonucleotide described herein (e.g., ASO SEQ ID NO: 19, 44, 28, 37, 67, or 80).

FIG. 2 illustrates knockdown of the expression of KRAS protein (G12C mutation or wildtype KRAS) and downstream biomarkers (pERK, pS6 and cPARP) mediated by the oligonucleotide described herein (e.g., ASO SEQ ID NO: 28) in two different cell lines (NCI-H358 and A375).

FIG. 3 illustrates knockdown of the expression of mutated KRAS protein and downstream biomarkers (pERK, pS6 and cPARP) in NCI-H358 cell line mediated by the oligonucleotide described herein (e.g., ASO SEQ ID NO:28 or ASO SEQ ID NO:67).

FIG. 4 illustrates three-dimensional (3D) cell proliferation inhibition due to inhibiting expression of mutated KRAS by contacting the cell harboring the KRAS mutation (NCI-H358) with the oligonucleotide described herein (e.g., ASO SEQ ID NO: 19, 44, 28, 67, 72, 74, 75, 76, 77, 78, 79, 80 or 81).

FIG. 5 illustrates knockdown of KRAS mRNA encoding a mutated KRAS protein (NCI-H441) comprising the G12V mutation mediated by the oligonucleotide described herein (e.g., ASO SEQ ID NO: 19, 44, 77, 78, 79, 80, 81, 28, 67, 37, 70 and 72).

FIG. 6 illustrates knockdown of the expression of mutated KRAS protein and downstream biomarkers (pERK, pAKT, and pS6) in LCLC97TM1 (cell harboring the G12V mutation) and wildtype KRAS (A375) mediated by the ASO SEQ ID NO: 80.

FIG. 7 illustrates knockdown of the expression of mutated KRAS protein and downstream biomarkers (pERK, pAKT, and pS6) in LCLC97TM1 (cell harboring the G12V mutation) mediated by the ASO SEQ ID NO: 81.

FIG. 8 illustrates 3D cell proliferation inhibition by inhibiting expression of mutated KRAS by contacting the cell harboring the KRAS mutation (LCLC97TM1 cell, NCI-H441 cell, or CFPAC-1 cell having the G12V mutation) with the oligonucleotide described herein (e.g., ASO SEQ ID NO: 1, 19, 44, 28, 67, 72, 74, 75, 76, 77, 78, 79, 80 or 81).

FIG. 9 illustrates 3D cell proliferation inhibition by inhibiting expression of mutated KRAS by contacting the cell harboring the KRAS mutation (NCI-H2009 cells harboring the G12A mutation) with the oligonucleotide described herein (e.g., ASO SEQ ID NO: 1, 19, 44, 28, 67, 72, 73, 74, 75, 76, 77, 79, 80 or 81).

FIG. 10 illustrates knockdown of KRAS mRNA encoding a mutated KRAS protein comprising the G12D mutation (Panc1 and AsPC1) mediated by the oligonucleotide described herein (e.g., ASO SEQ ID NO: 19, 28, 37, 67, 78 and 81).

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

### Overview

Described herein are compositions and methods for modulating gene or signaling pathway expressions. Also described herein are composition and methods for treating a disease or condition by modulating the gene or signaling pathway expression associated with the disease or condition. In some aspects, the composition comprises at least one oligonucleotide that, upon delivered into a cell, binds to an endogenous nucleic acid, which leads to the degradation of the target nucleic acid. In some aspects, described herein is a method for utilizing the composition or the oligonucleotide described herein. In some aspects, the methods treats the disease or condition by contacting a cell with the oligonucleotide to decrease the gene or signaling pathway expression associated with the disease or condition.

In some aspects, the oligonucleotide is an antisense oligonucleotide, where the oligonucleotide is complementary and binds to at least one endogenous nucleic acid (e.g., an mRNA). In some aspects, the binding of the oligonucleotide to the endogenous nucleic acid leads to degradation of the endogenous nucleic acid or blocking of translation of the target protein from the endogenous nucleic acid, hence decrease of the expression of the gene encoded by the endogenous nucleic acid. For example, the binding of the oligonucleotide to the endogenous nucleic acid comprising an mRNA creates a duplex nucleic acid molecule, which can then recruit endogenous nuclease for degradation of the mRNA.

In some aspects, the oligonucleotide comprises at least one gap segment. In some aspects, the oligonucleotide comprises at least one wing segment. In some aspects, the oligonucleotide comprises at least one gap segment flanked by two wing segments. For example, the oligonucleotide comprises a gap segment flanked by a 5′-wing segment and a 3′-wing segment. In some aspects, the gap segment or the wing segment comprises at least one chemical modification.

In some aspects, the gene modulated by the oligonucleotide is part of the signaling pathway. In some aspects, the signaling pathway is a KRAS signaling pathway. In some aspects, the KRAS signaling pathway comprises a KRAS-RAF-MEK-ERK signaling pathway. In some aspects, the KRAS signaling pathway comprises phosphoinositide 3-kinase (PI3K) signaling pathway, mitogen-activated protein kinase (MAPK) signaling pathway, or Ral guanine nucleotide exchange factor (Ral-GEF) signaling pathway. As such, In some aspects, the decreasing of the expression of the gene due to the binding of the oligonucleotide to the endogenous nucleic acid can further decrease a signaling pathway expression comprising the gene modulated by the oligonucleotide. In some aspects, the decreasing gene or signaling pathway expression leads to therapeutic effects for treating the disease or condition. In some aspects, the disease or condition is caused by increased gene or signaling pathway expression. In some aspects, the disease or condition described herein is caused by genetic mutations associated with the gene or signaling pathway.

### Compositions

Described herein, in some aspects, are compositions comprising at least one oligonucleotide described herein. In some aspects, the composition comprises at least two, three, four, five, six, seven, eight, nine, ten, or more oligonucleotides. In some aspects, the oligonucleotides comprise same or difference nucleic acid sequences. In some aspects, the oligonucleotide described herein is an antisense oligonucleotide for targeting and bind to an endogenous nucleic acid. In some aspects, the binding of the oligonucleotide to the endogenous nucleic acid recruits endogenous nuclease for degrading the endogenous nucleic acid. In some aspects, the degradation of the endogenous nucleic acid decreases expression of the gene encoded by the endogenous nucleic acid. In some aspects, the degradation of the endogenous nucleic acid can treat a disease or condition described herein.

In some aspects, the oligonucleotide comprises a length of at least five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, or more nucleic acid bases. In some aspects, the oligonucleotide comprises a length of at least 10, 11, 12, 13, 14, 15, 16, 17, 18, 19, or 20 nucleic acid bases. In some aspects, the oligonucleotide comprises 10 nucleic acid bases. In some aspects, the oligonucleotide comprises 11 nucleic acid bases. In some aspects, the oligonucleotide comprises 12 nucleic acid bases. In some aspects, the oligonucleotide comprises 13 nucleic acid bases. In some aspects, the oligonucleotide comprises 14 nucleic acid bases. In some aspects, the oligonucleotide comprises 15 nucleic acid bases. In some aspects, the oligonucleotide comprises 16 nucleic acid bases. In some aspects, the oligonucleotide comprises 17 nucleic acid bases. In some aspects, the oligonucleotide comprises 18 nucleic acid bases. In some aspects, the oligonucleotide comprises 19 nucleic acid bases. In some aspects, the oligonucleotide comprises 20 nucleic acid bases.

In some aspects, the oligonucleotide comprises at least one gap segment. In some aspects, the gap segment comprises at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, or more nucleic acid bases. In some aspects, the gap segment comprises at least one four, five, six, seven, right, nine, 10, 11, 12, 13, or 14 nucleic acid bases. In some aspects, the gap segment comprises four nucleic acid bases. In some aspects, the gap segment comprises five nucleic acid bases. In some aspects, the gap segment comprises six nucleic acid bases. In some aspects, the gap segment comprises seven nucleic acid bases. In some aspects, the gap segment comprises eight nucleic acid bases. In some aspects, the gap segment comprises nine nucleic acid bases. In some aspects, the gap segment comprises 10 nucleic acid bases. In some aspects, the gap segment comprises 11 nucleic acid bases. In some aspects, the gap segment comprises 12 nucleic acid bases. In some aspects, the gap segment comprises 13 nucleic acid bases. In some aspects, the gap segment comprises 14 nucleic acid bases.

In some aspects, the oligonucleotide comprises at least one wing segment. In some aspects, the at least one wing segment is a 5′-end wing segment that is covalently connected to the gap segment at the 5′-end of the gap segment. In some aspects, the at least one wing segment is a 3′-end wing segment that is covalently connected to the gap segment at the 3′-end of the gap segment. In some aspects, the gap segment is flanked by the wing segments at both the 5′-end and the 3′-end of the gap segment. In some aspects, the wing segment comprises at least one, two, three, four, five, six, seven, eight, nine, 10, 11, 12, or more nucleic acid bases. In some aspects, the wing segment comprises one nucleic acid base. In some aspects, the wing segment comprises two nucleic acid bases. In some aspects, the wing segment comprises three nucleic acid bases. In some aspects, the wing segment comprises four nucleic acid bases. In some aspects, the wing segment comprises five nucleic acid bases. In some aspects, the wing segment comprises six nucleic acid bases. In some aspects, the wing segment comprises seven nucleic acid bases. In some aspects, the wing segment comprises eight nucleic acid bases. In some aspects, the wing segment comprises nine nucleic acid bases. In some aspects, the wing segment comprises 10 nucleic acid bases.

In some aspects, the oligonucleotide comprises a 5′-end wing segment followed by a gap segment followed by a 3′-end wing segment. In such arrangement, the 5′-end wing segment comprises one, two, three, four, five, six, seven, eight, nine, 10, 11, 12, or more nucleic acid bases and the 3′-end wing segment comprises one, two, three, four, five, six, seven, eight, nine, 10, 11, 12, or more nucleic acid bases. In some aspects, the 5′-end wing segment and the 3′end wing segment comprises the same number of nucleic acid base. In some aspects, the 5′-end wing segment and the 3′end wing segment comprises a different number of nucleic acid bases. In some aspects, the 5′-end wing segment comprises one nucleic acid base. In some aspects, the 5′-end wing segment comprises two nucleic acid bases. In some aspects, the 5′-end wing segment comprises three nucleic acid bases. In some aspects, the 5′-end wing segment comprises four nucleic acid bases. In some aspects, the 5′-end wing segment comprises five nucleic acid bases. In some aspects, the 5′-end wing segment comprises six nucleic acid bases. In some aspects, the 5′-end wing segment comprises seven nucleic acid bases. In some aspects, the 5′-end wing segment comprises eight nucleic acid bases. In some aspects, the 5′-end wing segment comprises nine nucleic acid bases. In some aspects, the 5′-end wing segment comprises 10 nucleic acid bases. In some aspects, the 3′-end wing segment comprises one nucleic acid base. In some aspects, the 3′-end wing segment comprises two nucleic acid bases. In some aspects, the 3′-end wing segment comprises three nucleic acid bases. In some aspects, the 3′-end wing segment comprises four nucleic acid bases. In some aspects, the 3′-end wing segment comprises five nucleic acid bases. In some aspects, the 3′-end wing segment comprises six nucleic acid bases. In some aspects, the 3′-end wing segment comprises seven nucleic acid bases. In some aspects, the 3′-end wing segment comprises eight nucleic acid bases. In some aspects, the 3′-end wing segment comprises nine nucleic acid bases. In some aspects, the 3′-end wing segment comprises 10 nucleic acid bases.

In some aspects, the oligonucleotide comprises a 5′-end wing segment comprising one nucleic acid base and a 3′-end wing segment comprising one nucleic acid base. In some aspects, the oligonucleotide comprises a 5′-end wing segment comprising two nucleic acid bases and a 3′-end wing segment comprising two nucleic acid bases. In some aspects, the oligonucleotide comprises a 5′-end wing segment comprising three nucleic acid bases and a 3′-end wing segment comprising three nucleic acid bases. In some aspects, the oligonucleotide comprises a 5′-end wing segment comprising four nucleic acid bases and a 3′-end wing segment comprising four nucleic acid bases. In some aspects, the oligonucleotide comprises a 5′-end wing segment comprising five nucleic acid bases and a 3′-end wing segment comprising five nucleic acid bases. In some aspects, the oligonucleotide comprises a 5′-end wing segment comprising six nucleic acid bases and a 3′-end wing segment comprising six nucleic acid bases. In some aspects, the oligonucleotide comprises a 5′-end wing segment comprising seven nucleic acid bases and a 3′-end wing segment comprising seven nucleic acid bases. In some aspects, the oligonucleotide comprises a 5′-end wing segment comprising eight nucleic acid bases and a 3′-end wing segment comprising eight nucleic acid bases. In some aspects, the oligonucleotide comprises a 5′-end wing segment comprising nine nucleic acid bases and a 3′-end wing segment comprising nine nucleic acid bases. In some aspects, the oligonucleotide comprises a 5′-end wing segment comprising 10 nucleic acid bases and a 3′-end wing segment comprising 10 nucleic acid bases.

In some aspects, the oligonucleotide is an antisense oligonucleotide. In some aspects, the antisense oligonucleotide binds to a target nucleic acid. In some aspects, the target nucleic acid is an endogenous nucleic acid. In some aspects, the target nucleic acid comprises a nuclear RNA, a cytoplasmic RNA, or a mitochondrial RNA. In some aspects, the target RNA comprises an intergenic DNA (including, without limitation, heterochromatic DNA), a messenger RNA (mRNA), a pre-messenger RNA (pre-mRNA), a transfer RNA (tRNA), a ribosomal RNA (rRNA), a ribozyme, cDNA, a recombinant polynucleotide, a branched polynucleotide, a plasmid, a vector, an isolated DNA of a sequence, an isolated RNA of a sequence, a sgRNA, a oligonucleotide, a nucleic acid probe, a primer, an snRNA, a long non-coding RNA, a small RNA, a snoRNA, a siRNA, a miRNA, a tRNA-derived small RNA (tsRNA), an antisense RNA, an shRNA, or a small rDNA-derived RNA (srRNA). In some aspects, the oligonucleotide comprises a nucleic acid sequence that allows the oligonucleotide to bind to target nucleic acid by base pairing such as Watson Crick base pairing. Compositions and methods provided herein can be utilized to modulate expression of a gene or signaling pathway. Modulation can refer to altering the expression of a gene or portion thereof at one of various stages, with a view to alleviate a disease or condition associated with the gene or a mutation in the gene. Modulation can be mediated at the level of transcription or post-transcriptionally. Modulating transcription can correct aberrant expression of splice variants generated by a mutation in a gene. In some cases, compositions and methods provided herein can be utilized to regulate gene translation of a target. Modulation can refer to decreasing or knocking down the expression of a gene or portion thereof by decreasing the abundance of a transcript. The decreasing the abundance of a transcript can be mediated by decreasing the processing, splicing, turnover or stability of the transcript; or by decreasing the accessibility of the transcript by translational machinery such as ribosome. In some cases, an oligonucleotide described herein can facilitate a knockdown. A knockdown can reduce the expression of a target RNA. In some cases, a knockdown can be accompanied by modulating of an mRNA. In some cases, a knockdown can occur with substantially little to no modulating of an mRNA. In some instances, a knockdown can occur by targeting an untranslated region of the target RNA, such as a 3′ UTR, a 5′ UTR or both. In some cases, a knockdown can occur by targeting a coding region of the target RNA.

In some aspects, the oligonucleotide is an antisense oligonucleotide for targeting and binding any one of the genes described herein. In some aspects, the gene(s) being targeted and bound by the oligonucleotide is KRAS or mutated KRAS. In some embodiments, the mutated KRAS gene encodes a mutated KRAS protein comprising a G12C mutation. In some embodiments, the mutated KRAS gene encodes a mutated KRAS protein comprising a G12V mutation. In some embodiments, the mutated KRAS gene encodes a mutated KRAS protein comprising a G12A mutation. In some embodiments, the mutated KRAS gene encodes a mutated KRAS protein comprising a G12D mutation.

In some aspects, the oligonucleotide targets and binds to an mRNA of KRAS or an mRNA of mutated KRAS. In some aspects, the oligonucleotide comprises an nucleic acid sequence that is at least 70%, 75%, 80%, 85%, 90%, 95%, or 99% identical to any one of the nucleic acid sequence of Tables 7-9. In some aspects, the oligonucleotide comprises an nucleic acid sequence that is at least 70%, 75%, 80%, 85%, 90%, 95%, or 99% identical to any one SEQ ID NOs: 100-556, 24-43, 65-82, or 87. In some aspects, the oligonucleotide comprises an nucleic acid sequence that is at least 70%, 75%, 80%, 85%, 90%, 95%, or 99% identical to any one of SEQ ID NO: 129, SEQ ID NO: 213, SEQ ID NO: 214, SEQ ID NO: 215, SEQ ID NO: 216, SEQ ID NO: 217, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 392, SEQ ID NO: 393, SEQ ID NO: 394, SEQ ID NO: 399, SEQ ID NO: 400, SEQ ID NO: 401, SEQ ID NO: 402, SEQ ID NO: 427, SEQ ID NO: 428, SEQ ID NO: 429, SEQ ID NO: 430, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 494, SEQ ID NO: 495, SEQ ID NO: 496, SEQ ID NO: 497, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 18, SEQ ID NO: 19, or SEQ ID NO: 20. In some aspects, the oligonucleotide comprises an nucleic acid sequences that is at least 70%, 75%, 80%, 85%, 90%, 95%, or 99% identical to any one of SEQ ID NOS: 24-43, 65-82, or 87.

In some aspects, the oligonucleotide comprises at least one gap segment. In some aspects, the at least one gap segment comprises an nucleic acid sequence that is at least 70%, 75%, 80%, 85%, 90%, 95%, or 99% identical to at least a portion of any one of the nucleic acid sequence of SEQ ID NOs: 100-556, 24-43, 65-82, or 87. In some aspects, the at least one gap segment comprises an nucleic acid sequence that is at least 70%, 75%, 80%, 85%, 90%, 95%, or 99% identical to at least 5, 6, 7, 8, 9, 10 consecutive sequences of any one of the nucleic acid sequence of SEQ ID NOs: 100-556, 24-43, 65-82, or 87. In some aspects, the at least one gap segment comprises an nucleic acid sequence that is at least 70%, 75%, 80%, 85%, 90%, 95%, or 99% identical to at least a portion of, and/or at least 5, 6, 7, 8, 9, 10 consecutive sequences of SEQ ID NO: 129, SEQ ID NO: 213, SEQ ID NO: 214, SEQ ID NO: 215, SEQ ID NO: 216, SEQ ID NO: 217, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 392, SEQ ID NO: 393, SEQ ID NO: 394, SEQ ID NO: 399, SEQ ID NO: 400, SEQ ID NO: 401, SEQ ID NO: 402, SEQ ID NO: 427, SEQ ID NO: 428, SEQ ID NO: 429, SEQ ID NO: 430, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 494, SEQ ID NO: 495, SEQ ID NO: 496, SEQ ID NO: 497, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 18, SEQ ID NO: 19, or SEQ ID NO: 20.

In some aspects, the oligonucleotide comprising a nucleic acid sequence of any one of SEQ ID NOs: 18-20 can bind or preferentially bind to a wild type KRAS sequence. In some embodiments, the oligonucleotide comprising a nucleic acid sequence of any one of SEQ ID NOs: 24-33, 65-67, or 82 can bind or preferentially bind to a mutated KRAS sequence encoding a G12C mutation. In some embodiments, the oligonucleotide comprising a nucleic acid sequence of any one of SEQ ID NOs: 77-81 can bind or preferentially bind to a mutated KRAS sequence encoding a G12V mutation. In some embodiments, the oligonucleotide comprising a nucleic acid sequence of nay one of SEQ ID NOs: 72-76 or 87 can bind or preferentially bind to a mutated KRAS sequence encoding a G12A mutation. In some embodiments, the oligonucleotide comprising a nucleic acid sequence of any one of SEQ ID NOs: 34-43 or 68-71 can bind or preferentially bind to a mutated KRAS sequence encoding a G12D mutation.

In some aspects, the oligonucleotide described herein targets and binds an endogenous nucleic acid encoding a gene associated with the KRAS-RAF-MEK-ERK signaling pathway. In some aspects, the gene associated with the KRAS-RAF-MEK-ERK signaling pathway is KRAS. In some aspects, the gene associated with the KRAS-RAF-MEK-ERK signaling pathway is mutated KRAS. In some aspects, the oligonucleotide described herein modulates or affects the expression or activity of a gene in or associated with the KRAS-RAF-MEK-ERK signaling pathway. In some aspects, the gene associated with the KRAS-RAF-MEK-ERK signaling pathway is RAS. In some aspects, the gene associated with the KRAS-RAF-MEK-ERK signaling pathway is RAF. In some aspects, the gene associated with the KRAS-RAF-MEK-ERK signaling pathway is MEK. In some aspects, the gene associated with the KRAS-RAF-MEK-ERK signaling pathway is ERK.

In some aspects, the oligonucleotide described herein targets and binds an endogenous nucleic acid encoding a gene associated with the PI3K signaling pathway, the MAPK signaling pathway, or the Ral-GEF signaling pathway.

In some aspects, the oligonucleotide, upon binding to the endogenous nucleic acid, forms a duplex with the endogenous nucleic acid and recruits an endogenous nuclease for degrading the endogenous nucleic acid. In some aspects, the endogenous nuclease is a deoxyribonuclease. In some aspects, the endogenous nuclease is a ribonuclease. In some aspects, the ribonucleases is an endoribonuclease. In some aspects, the endoribonuclease comprises endoribonuclease or RNase A, P, H, I, III, T1, T2, U2, V1, PhyM, or V. In some aspects, the ribonuclease is an exoribonuclease. In some aspects, the exoribonuclease comprises RNase PH, II, R, D, or T. In some aspects, the nuclease comprises polynucleotide phosphorylase (PNPase), oligoribonuclease, exoribonuclease I, or exoribonuclease II. In some aspects, the ribonuclease recruited by the oligonucleotide binding to the endogenous nucleic acid is RNase H.

In some aspects, the oligonucleotide comprises at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more chemical modifications. In some aspects, the oligonucleotide comprises at least one gap segment comprising at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more chemical modifications. In some aspects, the oligonucleotide comprises at least one wing segment comprising at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more chemical modifications. In some aspects, the oligonucleotide comprises at least one gap segment and at least one wing segment comprising at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more chemical modifications.

In some aspects, the oligonucleotide described herein binds to an endogenous nucleic acid (e.g., a mRNA) encoding KRAS, where the binding of the oligonucleotide to the KRAS endogenous nucleic acid decreases the endogenous expression of KRAS in a cell by at least 10%, 20%, 30%, 40%, 50%, 60%, 70%, 80%, 90%, or more compared to the endogenous expression of KRAS not modulated by the oligonucleotide. In some aspects, the oligonucleotide described herein binds to an endogenous nucleic acid (e.g., a mRNA) encoding mutated KRAS, where the binding of the oligonucleotide to the mutated KRAS endogenous nucleic acid decreases the endogenous expression of mutated KRAS in a cell by at least 10%, 20%, 30%, 40%, 50%, 60%, 70%, 80%, 90%, or more compared to the endogenous expression of mutated KRAS not modulated by the oligonucleotide. In some embodiments, the mutated KRAS endogenous nucleic acid encodes a mutated KRAS protein comprising a G12C mutation, a G12V mutation, a G12A mutation, or a G12D mutation.

In some aspects, the oligonucleotide described herein binds to an endogenous nucleic acid (e.g., a mRNA) encoding KRAS, where the binding of the oligonucleotide to the KRAS endogenous nucleic acid decreases the endogenous expression of the KRAS-RAF-MEK-ERK signaling pathway, the PI3K signaling pathway, the MAPK signaling pathway, or the Ral-GEF signaling pathway in a cell by at least 10%, 20%, 30%, 40%, 50%, 60%, 70%, 80%, 90%, or more compared to the endogenous expression of the KRAS-RAF-MEK-ERK signaling pathway, the PI3K signaling pathway, the MAPK signaling pathway, or the Ral-GEF signaling pathway not modulated by the oligonucleotide.

In some aspects, the oligonucleotide described herein binds to an endogenous nucleic acid (e.g., a mRNA) encoding mutated KRAS, where the binding of the oligonucleotide to the mutated KRAS endogenous nucleic acid decreases the endogenous expression of a gene in, or an activity of, the KRAS-RAF-MEK-ERK signaling pathway, the PI3K signaling pathway, the MAPK signaling pathway, or the Ral-GEF signaling pathway in a cell by at least 10%, 20%, 30%, 40%, 50%, 60%, 70%, 80%, 90%, or more compared to the endogenous expression of a gene in, or an activity of the KRAS-RAF-MEK-ERK signaling pathway, the PI3K signaling pathway, the MAPK signaling pathway, or the Ral-GEF signaling pathway not modulated by the oligonucleotide.

In some aspects, the composition comprises at least two oligonucleotides, where a first oligonucleotide binds to the KRAS endogenous nucleic acid (e.g., a KRAS mRNA) and a second oligonucleotide binds to the mutated KRAS endogenous nucleic acid (e.g., a mutated KRAS mRNA). In some embodiments, the mutated KRAS endogenous nucleic acid encodes a mutated KRAS protein comprising a G12C mutation, a G12V mutation, a G12A mutation, or a G12D mutation.

In some aspects, the binding of the oligonucleotides to both KRAS and mutated KRAS endogenous nucleic acids decreases the endogenous expression of a gene in, or an activity of, the KRAS-RAF-MEK-ERK signaling pathway, the PI3K signaling pathway, the MAPK signaling pathway, or the Ral-GEF signaling pathway in a cell by at least 10%, 20%, 30%, 40%, 50%, 60%, 70%, 80%, 90%, or more compared to the endogenous expression of a gene in, or an activity of the KRAS-RAF-MEK-ERK signaling pathway, the PI3K signaling pathway, the MAPK signaling pathway, or the Ral-GEF signaling pathway not modulated by the oligonucleotide.

In some aspects, the composition is formulated for administration to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations. In some aspects, the composition is formulated into a dosage form. In some aspects, the composition is formulated to include at least one excipient. In some aspects, the excipient is a pharmaceutically acceptable excipient.

In some aspects, the composition comprising the oligonucleotide described herein treats a disease or condition by decreasing the expression of the gene or the signaling pathway associated with the disease or condition. In some aspects, the composition comprising the oligonucleotide describe herein treats the disease or condition described herein by directly decreasing the gene expression associated with disease or condition described herein. In some aspects, the composition comprising the oligonucleotide treats the disease or condition by decreasing the gene expression as part of a signaling pathway described herein. In some aspects, the composition comprising the oligonucleotide described herein treats a disease or condition by decreasing the endogenous KRAS expression. In some aspects, the composition comprising the oligonucleotide described herein treats a disease or condition by decreasing the endogenous mutated KRAS expression. In some aspects, the composition comprising the oligonucleotide described herein treats a disease or condition by decreasing both endogenous KRAS and mutated KRAS expressions. In some aspects, the composition comprising the oligonucleotide described herein treats a disease or condition by decreasing the endogenous KRAS expression. In some aspects, the composition comprising the oligonucleotide described herein treats a disease or condition by decreasing the endogenous the KRAS-RAF-MEK-ERK signaling pathway, the PI3K signaling pathway, the MAPK signaling pathway, or the Ral-GEF signaling pathway expression or activity. In some aspects, the disease or condition described herein is cancer.

### Chemical Modifications

Described herein, In some aspects, is an oligonucleotide comprising at least one chemical modification. In some aspects, the oligonucleotide is single-stranded. In some aspects, the oligonucleotide is an antisense oligonucleotide. In some aspects, the oligonucleotide comprises at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more chemical modifications. In some aspects, the oligonucleotide does not have an intramolecular structure feature. In some aspects, the oligonucleotide comprises at least one gap segment comprising at least one, two, three, four, five, six, seven, eight, nine, ten, 11, 12, 13, 14, 15, or more chemically modified nucleotides. In some aspects, the oligonucleotide comprises at least one wing segment comprising at least one, two, three, four, five, six, seven, eight, nine, ten, or more chemically modified nucleotides. In some aspects, the oligonucleotide comprises a 5′-end wing segment comprising at least one, two, three, four, five, six, seven, eight, nine, ten, or more chemically modified nucleotides. In some aspects, the oligonucleotide comprises a 3′-end wing segment comprising at least one, two, three, four, five, six, seven, eight, nine, ten, or more chemically modified nucleotides. In some aspects, the at least one wing segment is covalently fused to the 5′-end of the gap segment. In some aspects, the at least one wing segment is covalently fused to the 3′-end of the gap segment.

In some aspects, the oligonucleotide comprises at least one, two, three, four, five, six, seven, eight, nine, ten, 11, 12, 13, 14, 15, 16, 17, 18, 19, 20 or more chemically modified nucleotides at the 5′ end of the oligonucleotide. In some aspects, the oligonucleotide comprises at least one, two, three, four, five, six, seven, eight, nine, ten, 11, 12, 13, 14, 15, 16, 17, 18, 19, 20 or more chemically modified nucleotides at the 3′ end of the oligonucleotide. In some aspects, the oligonucleotide comprises at least one, two, three, four, five, six, seven, eight, nine, ten, 11, 12, 13, 14, 15, 16, 17, 18, 19, 20 or more chemically modified nucleotides at both the 5′ and the 3′ end of the oligonucleotide. In some aspects, the oligonucleotide comprises at least one chemical modification in the gap segment of the oligonucleotide. In some aspects, the oligonucleotide comprises at least one chemical modification in the nucleotide bases adjacent the gap segment. In some aspects, at least 10%, 20%, 30%, 40%, 50%, 60%, 70%, 80%, 90% of the bases or internucleotide linkage of the oligonucleotide comprises modifications. In some aspects, the oligonucleotide comprises 100% modified nucleotide bases.

In some aspects, chemical modification can occur at 3′OH, group, 5′OH group, at the backbone, at the sugar component, or at the nucleotide base. Chemical modification can include non-naturally occurring linker molecules of interstrand or intrastrand cross links. In one aspect, the chemically modified nucleic acid comprises modification of one or more of the 3′OH or 5′OH group, the backbone, the sugar component, or the nucleotide base, or addition of non-naturally occurring linker molecules. In some aspects, chemically modified backbone comprises a backbone other than a phosphodiester backbone. In some aspects, a modified sugar comprises a sugar other than deoxyribose (in modified DNA) or other than ribose (modified RNA). In some aspects, a modified base comprises a base other than adenine, guanine, cytosine, thymine or uracil. In some aspects, the oligonucleotide comprises at least one chemically modified base. In some instances, the comprises at least one, two, three, four, five, six, seven, eight, nine, 10, 15, 20, or more modified bases. In some cases, chemical modifications to the base moiety include natural and synthetic modifications of adenine, guanine, cytosine, thymine, or uracil, and purine or pyrimidine bases.

In some aspects, the at least one chemical modification of the oligonucleotide comprises a modification of any one of or any combination of 2′ modified nucleotide comprising 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA); modification of one or both of the non-linking phosphate oxygens in the phosphodiester backbone linkage; modification of one or more of the linking phosphate oxygens in the phosphodiester backbone linkage; modification of a constituent of the ribose sugar; replacement of the phosphate moiety with “dephospho” linkers; modification or replacement of a naturally occurring nucleobase; modification of the ribose-phosphate backbone; modification of 5′ end of polynucleotide; modification of 3′ end of polynucleotide; modification of the deoxyribose phosphate backbone; substitution of the phosphate group; modification of the ribophosphate backbone; modifications to the sugar of a nucleotide; modifications to the base of a nucleotide; or stereopure of nucleotide. Non limiting examples of chemical modification to the oligonucleotide can include: modification of one or both of non-linking or linking phosphate oxygens in the phosphodiester backbone linkage (e.g., sulfur (S), selenium (Se), BR3 (wherein R can be, e.g., hydrogen, alkyl, or aryl), C (e.g., an alkyl group, an aryl group, and the like), H, NR2, wherein R can be, e.g., hydrogen, alkyl, or aryl, or wherein R can be, e.g., alkyl or aryl); replacement of the phosphate moiety with “dephospho” linkers (e.g., replacement with methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo, or methyleneoxymethylimino); modification or replacement of a naturally occurring nucleobase with nucleic acid analog; modification of deoxyribose-phosphate or ribose-phosphate backbone (e.g., modifying the ribose-phosphate backbone to incorporate phosphorothioate, phosphonothioacetate, phosphoroselenates, boranophosphates, borano phosphate esters, hydrogen phosphonates, phosphonocarboxylate, phosphoroamidates, alkyl or aryl phosphonates, phosphonoacetate, or phosphotriesters; modification of 5′ end (e.g., 5′ cap or modification of 5′ cap —OH) or 3′ end of the nucleic acid sequence (3′ tail or modification of 3′ end —OH); substitution of the phosphate group with methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo, or methyleneoxymethylimino; modification of the ribophosphate backbone to incorporate morpholino (phosphorodiamidate morpholino oligomer PMO), thiomorpholino, cyclobutyl, pyrrolidine, or peptide nucleic acid (PNA) nucleoside surrogates; modifications to the sugar of a nucleotide to incorporate locked nucleic acid (LNA), unlocked nucleic acid (UNA), ethylene nucleic acid (ENA), constrained ethyl (cEt) sugar, or bridged nucleic acid (BNA); modification of a constituent of the ribose sugar (e.g., 2′-O-methyl, 2′-O-methoxy-ethyl (2′-MOE), 2′-fluoro, 2′-aminoethyl, 2′-deoxy-2′-fuloarabinou cleic acid, 2′-deoxy, 2′-O-methyl, 3′-phosphorothioate, 3′-phosphonoacetate (PACE), or 3′-phosphonothioacetate (thioPACE)); modification to the base of a nucleotide (of A, T, C, G, or U); and stereopure of nucleotide (e.g., S conformation of phosphorothioate or R conformation of phosphorothioate).

In some aspects, the chemical modification of the oligonucleotide comprises at least one substitution of one or both of non-linking phosphate oxygen atoms in a phosphodiester backbone linkage of the oligonucleotide. In some aspects, the at least one chemical modification of the oligonucleotide comprises a substitution of one or more of linking phosphate oxygen atoms in a phosphodiester backbone linkage of the oligonucleotide. A non-limiting example of a chemical modification of a phosphate oxygen atom is a sulfur atom. In some aspects, the chemical modification of the oligonucleotide comprises at least one chemical modification to a sugar of a nucleotide of the oligonucleotide. In some aspects, the chemical modification of the oligonucleotide comprises at least one chemical modification to the sugar of the nucleotide, where the chemical modification comprises at least one locked nucleic acid (LNA). In some aspects, the chemical modification of the oligonucleotide comprises at least one chemical modification to the sugar of the nucleotide of the oligonucleotide comprising at least one unlocked nucleic acid (UNA). In some aspects, the chemical modification of the oligonucleotide comprises at least one chemical modification to the sugar of the nucleotide of the oligonucleotide comprising at least one ethylene nucleic acid (ENA). In some aspects, the chemical modification of the oligonucleotide comprises at least one chemical modification to the sugar comprising a modification of a constituent of the sugar, where the sugar is a ribose sugar. In some aspects, the chemical modification of the oligonucleotide comprises at least one chemical modification to the constituent of the ribose sugar of the nucleotide of the oligonucleotide comprising a 2′-O-Methyl group. In some aspects, the chemical modification of the oligonucleotide comprises at least one chemical modification comprising replacement of a phosphate moiety of the oligonucleotide with a dephospho linker. In some aspects, the chemical modification of the oligonucleotide comprises at least one chemical modification of a phosphate backbone of the oligonucleotide. In some aspects, the oligonucleotide comprises a phosphothioate group. In some aspects, the chemical modification of the oligonucleotide comprises at least one chemical modification comprising a modification to a base of a nucleotide of the oligonucleotide. In some aspects, the chemical modification of the oligonucleotide comprises at least one chemical modification comprising an unnatural base of a nucleotide. In some aspects, the chemical modification of the oligonucleotide comprises at least one chemical modification comprising a morpholino group (e.g., a phosphorodiamidate morpholino oligomer, PMO), a cyclobutyl group, pyrrolidine group, or peptide nucleic acid (PNA) nucleoside surrogate. In some aspects, the chemical modification of the oligonucleotide comprises at least one chemical modification comprising at least one stereopure nucleic acid. In some aspects, the at least one chemical modification can be positioned proximal to a 5′ end of the oligonucleotide. In some aspects, the at least one chemical modification can be positioned proximal to a 3′ end of the oligonucleotide. In some aspects, the at least one chemical modification can be positioned proximal to both 5′ and 3′ ends of the oligonucleotide.

In some aspects, an oligonucleotide comprises a backbone comprising a plurality of sugar and phosphate moieties covalently linked together. In some cases, a backbone of an oligonucleotide comprises a phosphodiester bond linkage between a first hydroxyl group in a phosphate group on a 5′ carbon of a deoxyribose in DNA or ribose in RNA and a second hydroxyl group on a 3′ carbon of a deoxyribose in DNA or ribose in RNA.

In some aspects, a backbone of an oligonucleotide can lack a 5′ reducing hydroxyl, a 3′ reducing hydroxyl, or both, capable of being exposed to a solvent. In some aspects, a backbone of an oligonucleotide can lack a 5′ reducing hydroxyl, a 3′ reducing hydroxyl, or both, capable of being exposed to nucleases. In some aspects, a backbone of an oligonucleotide can lack a 5′ reducing hydroxyl, a 3′ reducing hydroxyl, or both, capable of being exposed to hydrolytic enzymes. In some instances, a backbone of an oligonucleotide can be represented as a polynucleotide sequence in a circular 2-dimensional format with one nucleotide after the other. In some instances, a backbone of an oligonucleotide can be represented as a polynucleotide sequence in a looped 2-dimensional format with one nucleotide after the other. In some cases, a 5′ hydroxyl, a 3′ hydroxyl, or both, are joined through a phosphorus-oxygen bond. In some cases, a 5′ hydroxyl, a 3′ hydroxyl, or both, are modified into a phosphoester with a phosphorus-containing moiety.

In some aspects, the oligonucleotide described herein comprises at least one chemical modification. A chemical modification can be a substitution, insertion, deletion, chemical modification, physical modification, stabilization, purification, or any combination thereof. In some cases, a modification is a chemical modification. Suitable chemical modifications comprise any one of: 5′ adenylate, 5′ guanosine-triphosphate cap, 5′N7-Methylguanosine-triphosphate cap, 5′triphosphate cap, 3′phosphate, 3′thiophosphate, 5′phosphate, 5′thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′deoxyribonucleoside analog purine, 2′deoxyribonucleoside analog pyrimidine, ribonucleoside analog, 2′-O-methyl ribonucleoside analog, sugar modified analogs, wobble/universal bases, fluorescent dye label, 2′fluoro RNA, 2′O-methyl RNA, methylphosphonate, phosphodiester DNA, phosphodiester RNA, phosphothioate DNA, phosphorothioate RNA, UNA, LNA, cEt, pseudouridine-5′-triphosphate, 5-methylcytidine-5′-triphosphate, 2-O-methyl-phosphorothioate or any combinations thereof.

In some cases, a modification can be permanent. In other cases, a modification can be transient. In some cases, multiple modifications are made to the oligonucleotide. the oligonucleotide modification can alter physio-chemical properties of a nucleotide, such as their conformation, polarity, hydrophobicity, chemical reactivity, base-pairing interactions, or any combination thereof.

A chemical modification can also be a phosphorothioate substitute. In some cases, a natural phosphodiester bond can be susceptible to rapid degradation by cellular nucleases and; a modification of internucleotide linkage using phosphorothioate (PS) bond substitutes can be more stable towards hydrolysis by cellular degradation. A modification can increase stability in a polynucleic acid. A modification can also enhance biological activity. In some cases, a phosphorothioate enhanced RNA polynucleic acid can inhibit RNase A, RNase T1, calf serum nucleases, or any combinations thereof. These properties can allow the use of PS-RNA polynucleic acids to be used in applications where exposure to nucleases is of high probability in vivo or in vitro. For example, phosphorothioate (PS) bonds can be introduced between the last 3-5 nucleotides at the 5′- or 3′-end of a polynucleic acid which can inhibit exonuclease degradation. In some cases, phosphorothioate bonds can be added throughout an entire polynucleic acid to reduce attack by endonucleases. In some aspects, the oligonucleotide described herein comprises at least one, two, three, four, five, six, seven, eight, nine, 10, 11, 12, 13, 14, 15, 16, 17, 18, 19, 20, 21, 22, 23, 24, 25, 26, 27, 28, 29, 30, 50, 100, or more internucleotide linkage comprising PS bond. In some aspects, the oligonucleotide described herein comprises only PS bond as the internucleotide linkage modification. In some aspects, all internucleotide linkages of the oligonucleotide described herein are fully PS-modified or include phosphorothioate internucleotide linkages. In some aspects, the oligonucleotide comprising PS bond as the internucleotide linkage modification comprises a 5′-end wing segment comprising one nucleic acid base. In some aspects, the oligonucleotide comprising PS bond as the internucleotide linkage modification comprises a 5′-end wing segment comprising two nucleic acid bases. In some aspects, the oligonucleotide comprising PS bond as the internucleotide linkage modification comprises a 5′-end wing segment comprising three nucleic acid bases. In some aspects, the oligonucleotide comprising PS bond as the internucleotide linkage modification comprises a 5′-end wing segment comprising four nucleic acid bases. In some aspects, the oligonucleotide comprising PS bond as the internucleotide linkage modification comprises a 5′-end wing segment comprising five nucleic acid bases. In some aspects, the oligonucleotide comprising PS bond as the internucleotide linkage modification comprises a 5′-end wing segment comprising six nucleic acid bases. In some aspects, the oligonucleotide comprising PS bond as the internucleotide linkage modification comprises a 5′-end wing segment comprising seven nucleic acid bases. In some aspects, the oligonucleotide comprising PS bond as the internucleotide linkage modification comprises a 5′-end wing segment comprising eight nucleic acid bases. In some aspects, the oligonucleotide comprising PS bond as the internucleotide linkage modification comprises a 5′-end wing segment comprising nine nucleic acid bases. In some aspects, the oligonucleotide comprising PS bond as the internucleotide linkage modification comprises a 5′-end wing segment comprising 10 nucleic acid bases. In some aspects, the oligonucleotide comprising PS bond as the internucleotide linkage modification comprises a 3′-end wing segment comprising one nucleic acid base. In some aspects, the oligonucleotide comprising PS bond as the internucleotide linkage modification comprises a 3′-end wing segment comprising two nucleic acid bases. In some aspects, the oligonucleotide comprising PS bond as the internucleotide linkage modification comprises a 3′-end wing segment comprising three nucleic acid bases. In some aspects, the oligonucleotide comprising PS bond as the internucleotide linkage modification comprises a 3′-end wing segment comprising four nucleic acid bases. In some aspects, the oligonucleotide comprising PS bond as the internucleotide linkage modification comprises a 3′-end wing segment comprising five nucleic acid bases. In some aspects, the oligonucleotide comprising PS bond as the internucleotide linkage modification comprises a 3′-end wing segment comprising six nucleic acid bases. In some aspects, the oligonucleotide comprising PS bond as the internucleotide linkage modification comprises a 3′-end wing segment comprising seven nucleic acid bases. In some aspects, the oligonucleotide comprising PS bond as the internucleotide linkage modification comprises a 3′-end wing segment comprising eight nucleic acid bases. In some aspects, the oligonucleotide comprising PS bond as the internucleotide linkage modification comprises a 3′-end wing segment comprising nine nucleic acid bases. In some aspects, the oligonucleotide comprising PS bond as the internucleotide linkage modification comprises a 3′-end wing segment comprising 10 nucleic acid bases.

In some aspects, the oligonucleotide comprising PS bond as the internucleotide linkage modification comprises a 5′-end wing segment comprising one nucleic acid base and a 3′-end wing segment comprising one nucleic acid base. In some aspects, the oligonucleotide comprising PS bond as the internucleotide linkage modification comprises a 5′-end wing segment comprising two nucleic acid bases and a 3′-end wing segment comprising two nucleic acid bases. In some aspects, the oligonucleotide comprising PS bond as the internucleotide linkage modification comprises a 5′-end wing segment comprising three nucleic acid bases and a 3′-end wing segment comprising three nucleic acid bases. In some aspects, the oligonucleotide comprising PS bond as the internucleotide linkage modification comprises a 5′-end wing segment comprising four nucleic acid bases and a 3′-end wing segment comprising four nucleic acid bases. In some aspects, the oligonucleotide comprising PS bond as the internucleotide linkage modification comprises a 5′-end wing segment comprising five nucleic acid bases and a 3′-end wing segment comprising five nucleic acid bases. In some aspects, the oligonucleotide comprising PS bond as the internucleotide linkage modification comprises a 5′-end wing segment comprising six nucleic acid bases and a 3′-end wing segment comprising six nucleic acid bases. In some aspects, the oligonucleotide comprising PS bond as the internucleotide linkage modification comprises a 5′-end wing segment comprising seven nucleic acid bases and a 3′-end wing segment comprising seven nucleic acid bases. In some aspects, the oligonucleotide comprising PS bond as the internucleotide linkage modification comprises a 5′-end wing segment comprising eight nucleic acid bases and a 3′-end wing segment comprising eight nucleic acid bases. In some aspects, the oligonucleotide comprising PS bond as the internucleotide linkage modification comprises a 5′-end wing segment comprising nine nucleic acid bases and a 3′-end wing segment comprising nine nucleic acid bases. In some aspects, the oligonucleotide comprising PS bond as the internucleotide linkage modification comprises a 5′-end wing segment comprising 10 nucleic acid bases and a 3′-end wing segment comprising 10 nucleic acid bases.

In some aspects, the oligonucleotide comprises a 5′-end wing segment comprising one nucleic acid base, a gapmer, and a 3′-end wing segment comprising one nucleic acid base, where the internucleotide linkages of the oligonucleotide joining the 5′-end wing segment, the gapmer, and the 3′-end wing segment comprises only PS bonds. In some aspects, the oligonucleotide comprises a 5′-end wing segment comprising two nucleic acid bases, a gapmer, and a 3′-end wing segment comprising two nucleic acid bases, where the internucleotide linkages of the oligonucleotide joining the 5′-end wing segment, the gapmer, and the 3′-end wing segment comprises only PS bonds. In some aspects, the oligonucleotide comprises a 5′-end wing segment comprising three nucleic acid bases, a gapmer, and a 3′-end wing segment comprising three nucleic acid bases, where the internucleotide linkages of the oligonucleotide joining the 5′-end wing segment, the gapmer, and the 3′-end wing segment comprises only PS bonds. In some aspects, the oligonucleotide comprises a 5′-end wing segment comprising four nucleic acid bases, a gapmer, and a 3′-end wing segment comprising four nucleic acid bases, where the internucleotide linkages of the oligonucleotide joining the 5′-end wing segment, the gapmer, and the 3′-end wing segment comprises only PS bonds. In some aspects, the oligonucleotide comprises a 5′-end wing segment comprising five nucleic acid bases, a gapmer, and a 3′-end wing segment comprising five nucleic acid bases, where the internucleotide linkages of the oligonucleotide joining the 5′-end wing segment, the gapmer, and the 3′-end wing segment comprises only PS bonds. In some aspects, the oligonucleotide comprises a 5′-end wing segment comprising six nucleic acid bases, a gapmer, and a 3′-end wing segment comprising six nucleic acid bases, where the internucleotide linkages of the oligonucleotide joining the 5′-end wing segment, the gapmer, and the 3′-end wing segment comprises only PS bonds. In some aspects, the oligonucleotide comprises a 5′-end wing segment comprising seven nucleic acid bases, a gapmer, and a 3′-end wing segment comprising seven nucleic acid bases, where the internucleotide linkages of the oligonucleotide joining the 5′-end wing segment, the gapmer, and the 3′-end wing segment comprises only PS bonds. In some aspects, the oligonucleotide comprises a 5′-end wing segment comprising eight nucleic acid bases, a gapmer, and a 3′-end wing segment comprising eight nucleic acid bases, where the internucleotide linkages of the oligonucleotide joining the 5′-end wing segment, the gapmer, and the 3′-end wing segment comprises only PS bonds. In some aspects, the oligonucleotide comprises a 5′-end wing segment comprising nine nucleic acid bases, a gapmer, and a 3′-end wing segment comprising nine nucleic acid bases, where the internucleotide linkages of the oligonucleotide joining the 5′-end wing segment, the gapmer, and the 3′-end wing segment comprises only PS bonds. In some aspects, the oligonucleotide comprises a 5′-end wing segment comprising 10 nucleic acid bases, a gapmer, and a 3′-end wing segment comprising 10 nucleic acid bases, where the internucleotide linkages of the oligonucleotide joining the 5′-end wing segment, the gapmer, and the 3′-end wing segment comprises only PS bonds.

In some aspects, the oligonucleotide comprising the 5′-end wing segment, a gapmer, the 3′-end wing segment, and PS bond as internucleotide linkage comprises a nucleic acid sequence that is at least 70%, 75%, 80%, 85%, 90%, 95%, or 99% identical to SEQ ID NOs: 100-556 In some aspects, the oligonucleotide comprising the 5′-end wing segment, a gapmer, the 3′-end wing segment, and PS bond as internucleotide linkage comprises a nucleic acid sequence that is at least 70%, 75%, 80%, 85%, 90%, 95%, or 99% identical to SEQ ID NO: 129, SEQ ID NO: 213, SEQ ID NO: 214, SEQ ID NO: 215, SEQ ID NO: 216, SEQ ID NO: 217, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 392, SEQ ID NO: 393, SEQ ID NO: 394, SEQ ID NO: 399, SEQ ID NO: 400, SEQ ID NO: 401, SEQ ID NO: 402, SEQ ID NO: 427, SEQ ID NO: 428, SEQ ID NO: 429, SEQ ID NO: 430, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 494, SEQ ID NO: 495, SEQ ID NO: 496, SEQ ID NO: 497, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 18, SEQ ID NO: 19, or SEQ ID NO: 20.

In some aspects, the oligonucleotide comprises the 5′-end wing segment, a gapmer, the 3′-end wing segment, and PS bond as internucleotide linkage, where the gapmer comprises a nucleic acid sequence that is at least 70%, 75%, 80%, 85%, 90%, 95%, or 99% identical to any one of SEQ ID NOs: 100-556, 24-43, 65-82, or 87. In some aspects, the oligonucleotide comprises the 5′-end wing segment, a gapmer, the 3′-end wing segment, and PS bond as internucleotide linkage, where the gapmer comprises a nucleic acid sequence that is at least 70%, 75%, 80%, 85%, 90%, 95%, or 99% identical to SEQ ID NO: 129, SEQ ID NO: 213, SEQ ID NO: 214, SEQ ID NO: 215, SEQ ID NO: 216, SEQ ID NO: 217, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 392, SEQ ID NO: 393, SEQ ID NO: 394, SEQ ID NO: 399, SEQ ID NO: 400, SEQ ID NO: 401, SEQ ID NO: 402, SEQ ID NO: 427, SEQ ID NO: 428, SEQ ID NO: 429, SEQ ID NO: 430, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 494, SEQ ID NO: 495, SEQ ID NO: 496, SEQ ID NO: 497, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 18, SEQ ID NO: 19, or SEQ ID NO: 20.

In some instances, chemical modifications to enhance guide stability, synthesis, localization, intracellular retention, or lengthen half-lives may not be genetically encodable. An oligonucleotide can be circular, substantially circular, or otherwise linked in a contiguous fashion (e.g. can be arranged as a loop) and can also retain a substantially similar secondary structure as a substantially similar oligonucleotide that may not be circular or may not be a loop.

### Modification of Phosphate Backbone

In some aspects, the chemical modification comprises modification of one or both of the non-linking phosphate oxygens in the phosphodiester backbone linkage or modification of one or more of the linking phosphate oxygens in the phosphodiester backbone linkage. As used herein, “alkyl” is meant to refer to a saturated hydrocarbon group which is straight-chained or branched. Example alkyl groups include methyl (Me), ethyl (Et), propyl (e.g., n-propyl or isopropyl), butyl (e.g., n-butyl, isobutyl, or t-butyl), or pentyl (e.g., n-pentyl, isopentyl, or neopentyl). An alkyl group can contain from 1 to about 20, from 2 to about 20, from 1 to about 12, from 1 to about 8, from 1 to about 6, from 1 to about 4, or from 1 to about 3 carbon atoms. As used herein, “aryl” refers to monocyclic or polycyclic (e.g., having 2, 3, or 4 fused rings) aromatic hydrocarbons such as, for example, phenyl, naphthyl, anthracenyl, phenanthrenyl, indanyl, or indenyl. In some aspects, aryl groups have from 6 to about 20 carbon atoms. As used herein, “alkenyl” refers to an aliphatic group containing at least one double bond. As used herein, “alkynyl” refers to a straight or branched hydrocarbon chain containing 2-12 carbon atoms and characterized in having one or more triple bonds. Examples of alkynyl groups can include ethynyl, propargyl, or 3-hexynyl. “Arylalkyl”

or “aralkyl” refers to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups. “Cycloalkyl” refers to a cyclic, bicyclic, tricyclic, or polycyclic non-aromatic hydrocarbon groups having 3 to 12 carbons. Examples of cycloalkyl moieties include, but are not limited to, cyclopropyl, cyclopentyl, and cyclohexyl. “Heterocyclyl” refers to a monovalent radical of a heterocyclic ring system. Representative heterocyclyls include, without limitation, tetrahydrofuranyl, tetrahydrothienyl, pyrrolidinyl, pyrrolidonyl, piperidinyl, pyrrolinyl, piperazinyl, dioxanyl, dioxolanyl, diazepinyl, oxazepinyl, thiazepinyl, and morpholinyl. “Heteroaryl” refers to a monovalent radical of a heteroaromatic ring system. Examples of heteroaryl moieties can include imidazolyl, oxazolyl, thiazolyl, triazolyl, pyrrolyl, furanyl, indolyl, thiophenyl pyrazolyl, pyridinyl, pyrazinyl, pyridazinyl, pyrimidinyl, indolizinyl, purinyl, naphthyridinyl, quinolyl, and pteridinyl.

In some aspects, the phosphate group of a chemically modified nucleotide can be modified by replacing one or more of the oxygens with a different substituent. In some aspects, the chemically modified nucleotide can include replacement of an unmodified phosphate moiety with a modified phosphate as described herein. In some aspects, the modification of the phosphate backbone can include alterations that result in either an uncharged linker or a charged linker with unsymmetrical charge distribution. Examples of modified phosphate groups can include phosphorothioate, phosphonothioacetate, phosphoroselenates, boranophosphates, boranophosphate esters, hydrogen phosphonates, phosphoroamidates, alkyl or aryl phosphonates and phosphotriesters. In some aspects, one of the non-bridging phosphate oxygen atoms in the phosphate backbone moiety can be replaced by any of the following groups: sulfur (S), selenium (Se), BR3 (wherein R can be, e.g., hydrogen, alkyl, or aryl), C (e.g., an alkyl group, an aryl group, and the like), H, NR2 (wherein R can be, e.g., hydrogen, alkyl, or aryl), or (wherein R can be, e.g., alkyl or aryl). The phosphorous atom in an unmodified phosphate group can be achiral. However, replacement of one of the non-bridging oxygens with one of the above atoms or groups of atoms can render the phosphorous atom chiral. A phosphorous atom in a phosphate group modified in this way is a stereogenic center. The stereogenic phosphorous atom can possess either the “R” configuration (herein Rp) or the “S” configuration (herein Sp). In some cases, the oligonucleotide comprises stereopure nucleotides comprising S conformation of phosphorothioate or R conformation of phosphorothioate. In some aspects, the chiral phosphate product is present in a diastereomeric excess of 50%, 60%, 70%, 80%, 90%, or more. In some aspects, the chiral phosphate product is present in a diastereomeric excess of 95%. In some aspects, the chiral phosphate product is present in a diastereomeric excess of 96%. In some aspects, the chiral phosphate product is present in a diastereomeric excess of 97%. In some aspects, the chiral phosphate product is present in a diastereomeric excess of 98%. In some aspects, the chiral phosphate product is present in a diastereomeric excess of 99%. In some aspects, both non-bridging oxygens of phosphorodithioates can be replaced by sulfur. The phosphorus center in the phosphorodithioates can be achiral which precludes the formation of oligoribonucleotide diastereomers. In some aspects, modifications to one or both non-bridging oxygens can also include the replacement of the non-bridging oxygens with a group independently selected from S, Se, B, C, H, N, and OR (R can be, e.g., alkyl or aryl). In some aspects, the phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either or both of the linking oxygens.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S).

Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and can be used in any combination. Other non-phosphate linkages may also be used.

In some aspects, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) is attached to the sugar or sugar analog moiety in and can be a monophosphate, diphosphate, triphosphate, alkylphosphonate, phosphorothioate, phosphorodithioate, phosphoramidate or the like.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos. A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. It is also understood in a nucleotide substitute that both the sugar and the phosphate moieties of the nucleotide can be replaced, by for example an amide type linkage (aminoethylglycine) (PNA). It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1-di-O-hexadecyl-rac-glycero-S—H-phosphonate, a polyamine or a polyethylene glycol chain, or adamantane acetic acid, a palmityl moiety, or an octadecylamine or hexylamino-carbonyl-oxy cholesterol moiety.

In some aspects, the chemical modification described herein comprises modification of a phosphate backbone. In some aspects, the oligonucleotide described herein comprises at least one chemically modified phosphate backbone. Exemplary chemically modification of the phosphate group or backbone can include replacing one or more of the oxygens with a different substituent. Furthermore, the modified nucleotide present in the oligonucleotide can include the replacement of an unmodified phosphate moiety with a modified phosphate as described herein. In some aspects, the modification of the phosphate backbone can include alterations resulting in either an uncharged linker or a charged linker with unsymmetrical charge distribution. Exemplary modified phosphate groups can include, phosphorothioate, phosphonothioacetate, phosphoroselenates, borano phosphates, borano phosphate esters, hydrogen phosphonates, phosphoroamidates, alkyl or aryl phosphonates and phosphotriesters. In some aspects, one of the non-bridging phosphate oxygen atoms in the phosphate backbone moiety can be replaced by any of the following groups: sulfur (S), selenium (Se), BR₃ (wherein R can be, e.g., hydrogen, alkyl, or aryl), C (e.g., an alkyl group, an aryl group, and the like), H, NR₂ (wherein R can be, e.g., hydrogen, alkyl, or aryl), or (wherein R can be, e.g., alkyl or aryl). The phosphorous atom in an unmodified phosphate group is achiral. However, replacement of one of the non-bridging oxygens with one of the above atoms or groups of atoms can render the phosphorous atom chiral; that is to say that a phosphorous atom in a phosphate group modified in this way is a stereogenic center. The stereogenic phosphorous atom can possess either the “R” configuration (herein Rp) or the “S” configuration (herein Sp). In such case, the chemically modified oligonucleotide can be stereopure (e.g. S or R confirmation). In some cases, the chemically modified oligonucleotide comprises stereopure phosphate modification. For example, the chemically modified oligonucleotide comprises S conformation of phosphorothioate or R conformation of phosphorothioate.

Phosphorodithioates have both non-bridging oxygens replaced by sulfur. The phosphorus center in the phosphorodithioates is achiral which precludes the formation of oligoribonucleotide diastereomers. In some aspects, modifications to one or both non-bridging oxygens can also include the replacement of the non-bridging oxygens with a group independently selected from S, Se, B, C, H, N, and OR (R can be, e.g., alkyl or aryl).

The phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

### Replacement of Phosphate Moiety

In some aspects, at least one phosphate group of the oligonucleotide can be chemically modified. In some aspects, the phosphate group can be replaced by non-phosphorus containing connectors. In some aspects, the phosphate moiety can be replaced by dephospho linker. In some aspects, the charge phosphate group can be replaced by a neutral group. In some cases, the phosphate group can be replaced by methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. In some aspects, nucleotide analogs described herein can also be modified at the phosphate group. Modified phosphate group can include modification at the linkage between two nucleotides with phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates (e.g. 3′-amino phosphoramidate and aminoalkylphosphoramidates), thionophosphoramidates, thionoalkylphosphonates, thionoalkylphosphotriesters, and boranophosphates. The phosphate or modified phosphate linkage between two nucleotides can be through a 3′-5′ linkage or a 2′-5′ linkage, and the linkage contains inverted polarity such as 3′-5′ to 5′-3′ or 2′-5′ to 5′-2′.

### Substitution of Phosphate Group

In some aspects, the chemical modification described herein comprises modification by replacement of a phosphate group. In some aspects, the oligonucleotide described herein comprises at least one chemically modification comprising a phosphate group substitution or replacement. Exemplary phosphate group replacement can include non-phosphorus containing connectors. In some aspects, the phosphate group substitution or replacement can include replacing charged phosphate group can by a neutral moiety. Exemplary moieties which can replace the phosphate group can include methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

### Modification of the Ribophosphate Backbone

In some aspects, the chemical modification described herein comprises modifying ribophosphate backbone of the oligonucleotide. In some aspects, the oligonucleotide described herein comprises at least one chemically modified ribophosphate backbone. Exemplary chemically modified ribophosphate backbone can include scaffolds that can mimic nucleic acids can also be constructed wherein the phosphate linker and ribose sugar are replaced by nuclease resistant nucleoside or nucleotide surrogates. In some aspects, the nucleobases can be tethered by a surrogate backbone. Examples can include morpholino such as a phosphorodiamidate morpholino oligomer (PMO), cyclobutyl, pyrrolidine and peptide nucleic acid (PNA) nucleoside surrogates.

### Modification of Sugar

In some aspects, the chemical modification described herein comprises modification of sugar. In some aspects, the oligonucleotide described herein comprises at least one chemically modified sugar. Exemplary chemically modified sugar can include 2′ hydroxyl group (OH) modified or replaced with a number of different “oxy” or “deoxy” substituents. In some aspects, modifications to the 2′ hydroxyl group can enhance the stability of the nucleic acid since the hydroxyl can no longer be deprotonated to form a 2′-alkoxide ion. The 2′-alkoxide can catalyze degradation by intramolecular nucleophilic attack on the linker phosphorus atom. Examples of “oxy”-2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR, wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some aspects, the “oxy”-2′ hydroxyl group modification can include (LNA, in which the 2′ hydroxyl can be connected, e.g., by a Ci₋₆ alkylene or Cj-6 heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some aspects, the “oxy”-2′ hydroxyl group modification can include the methoxyethyl group (MOE), (OCH₂CH₂OCH₃, e.g., a PEG derivative). In some cases, the deoxy modifications can include hydrogen (i.e., deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which can be optionally substituted with e.g., an amino as described herein. In some instances, the sugar group can also contain one or more carbons that possess the opposite stereochemical configuration than that of the corresponding carbon in ribose. Thus, a modified nucleic acid can include nucleotides containing e.g., arabinose, as the sugar. The nucleotide “monomer” can have an alpha linkage at the F position on the sugar, e.g., alpha-nucleosides. The modified nucleic acids can also include “abasic” sugars, which lack a nucleobase at C—. The abasic sugars can also be further modified at one or more of the constituent sugar atoms. The modified nucleic acids can also include one or more sugars that are in the L form, e.g. L-nucleosides. In some aspects, the oligonucleotide described herein includes the sugar group ribose, which is a 5-membered ring having an oxygen. Exemplary modified nucleosides and modified nucleotides can include replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). In some aspects, the modified nucleotides can include multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid. In some aspects, the modifications to the sugar of the oligonucleotide comprises modifying the oligonucleotide to include locked nucleic acid (LNA), unlocked nucleic acid (UNA), ethylene nucleic acid (ENA), constrained ethyl (cEt) sugar, or bridged nucleic acid (BNA).

### Modification of a Constituent of the Ribose Sugar

In some aspects, the oligonucleotide described herein comprises at least one chemical modification of a constituent of the ribose sugar. In some aspects, the chemical modification of the constituent of the ribose sugar can include 2′-O-methyl, 2′-O-methoxyethyl (2′-O-MOE), 2′-fluoro, 2′-aminoethyl, 2′-deoxy-2′-fuloarabinou-cleic acid, 2′-deoxy, 2′-deoxy-2′-fluoro, 2′-O-methyl, 3′-phosphorothioate, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA) 3′-phosphonoacetate (PACE), or 3′-phosphonothioacetate (thioPACE). In some aspects, the chemical modification of the constituent of the ribose sugar comprises unnatural nucleic acid. In some instances, the unnatural nucleic acids include modifications at the 5′-position and the 2′-position of the sugar ring, such as 5′-CH₂-substituted 2′-O-protected nucleosides. In some cases, unnatural nucleic acids include amide linked nucleoside dimers have been prepared for incorporation into oligonucleotides wherein the 3′ linked nucleoside in the dimer (5′ to 3′) comprises a 2′-OCH₃ and a 5′-(S)—CH₃. Unnatural nucleic acids can include 2′-substituted 5′-CH₂ (or O) modified nucleosides. Unnatural nucleic acids can include 5′-methylenephosphonate DNA and RNA monomers, and dimers. Unnatural nucleic acids can include 5′-phosphonate monomers having a 2′-substitution and other modified 5′-phosphonate monomers. Unnatural nucleic acids can include 5′-modified methylenephosphonate monomers. Unnatural nucleic acids can include analogs of 5′ or 6′-phosphonate ribonucleosides comprising a hydroxyl group at the 5′ and/or 6′-position. Unnatural nucleic acids can include 5′-phosphonate deoxyribonucleoside monomers and dimers having a 5′-phosphate group. Unnatural nucleic acids can include nucleosides having a 6′-phosphonate group wherein the 5′ or/and 6′-position is unsubstituted or substituted with a thio-tert-butyl group (SC(CH₃)₃) (and analogs thereof); a methyleneamino group (CH₂NH₂) (and analogs thereof) or a cyano group (CN) (and analogs thereof).

In some aspects, unnatural nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids; replacement of the ribosyl ring oxygen atom with S, N(R), or C(R₁)(R₂) (R═H, C₁-C₁₂ alkyl or a protecting group); and combinations thereof.

In some instances, the oligonucleotide described herein comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety can be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol-like ribonucleosides.

Modifications to the sugar moiety include natural modifications of the ribose and deoxy ribose as well as unnatural modifications. Sugar modifications include, but are not limited to, the following modifications at the 2′ position: OH; F; O—, S—, or N-alkyl; O—, S—, or N-alkenyl; O-, S- or N-alkynyl; or O-alkyl-O-alkyl, wherein the alkyl, alkenyl and alkynyl can be substituted or unsubstituted C₁ to C₁₀, alkyl or C₂ to C₁₀ alkenyl and alkynyl. 2′ sugar modifications also include but are not limited to —O[(CH₂)ₙO]ₘ CH₃, —O(CH₂)ₙOCH₃, —O(CH₂)ₙNH₂, —O(CH₂)ₙCH₃, —O(CH₂)ₙONH₂, and —O(CH₂)ₙON[(CH₂)ₙ CH₃)]₂, where n and m are from 1 to about 10. Other chemical modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂ CH₃, ONO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Chemically modified sugars also include those that contain modifications at the bridging ring oxygen, such as CH₂ and S. Nucleotide sugar analogs can also have sugar mimetics such as cyclobutyl moieties in place of the pentofuranosyl sugar. Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′ (LNA); 4′-(CH₂)—S-2′; 4′—(CH₂)₂—O-2′ (ENA); 4′-CH(CH₃)—O-2′ and 4′-CH(CH₂OCH₃)—O-2′, and analogs thereof; 4′-C(CH₃)(CH₃)—O-2′ and analogs thereof.

### Modifications on the Base of Nucleotide

In some aspects, the chemical modification described herein comprises modification of the base of nucleotide (e.g. the nucleobase). Exemplary nucleobases can include adenine (A), thymine (T), guanine (G), cytosine (C), and uracil (U). These nucleobases can be modified or replaced to in the oligonucleotide described herein. The nucleobase of the nucleotide can be independently selected from a purine, a pyrimidine, a purine or pyrimidine analog. In some aspects, the nucleobase can be naturally-occurring or synthetic derivatives of a base.

In some aspects, the chemical modification described herein comprises modifying an uracil. In some aspects, the oligonucleotide described herein comprises at least one chemically modified uracil. Exemplary chemically modified uracil can include pseudouridine, pyridin-4-one ribonucleoside, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy-uridine, 5-aminoallyl-uridine, 5-halo-uridine (e.g., 5-iodo-uridine or 5-bromo-uridine), 3-methyl-uridine, 5-methoxy-uridine, uridine 5-oxyacetic acid, uridine 5-oxyacetic acid methyl ester, 5-carboxymethyl-uridine, 1-carboxymethyl-pseudouridine, 5-carboxyhydroxymethyl-uridine, 5-carboxyhydroxymethyl-uridine methyl ester, 5-methoxycarbonylmethyl-uridine, 5-methoxycarbonylmethyl-2-thio-uridine, 5-aminomethyl-2-thio-uridine, 5-methylaminomethyl-uridine, 5-methylaminomethyl-2-thio-uridine, 5-methylaminomethyl-2-seleno-uridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl-pseudouridine, 5-taurinomethyl-2-thio-uridine, 1-taurinomethyl-4-thio-pseudouridine, 5-methyl-uridine, 1 methyl-pseudouridine, 5-methyl-2-thio-uridine, 1-methyl-4-thio-pseudouridine, 4-thio-1-methyl-pseudouridine, 3-methyl-pseudouridine, 2-thio-1-methyl-pseudouridine, 1-methyl-1-deaza-pseudouridine, 2-thio-1-methyl-1-deaza-pseudouridine, dihydroundine, dihydropseudoundine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine, 1-methyl-3-(3-amino-3-carboxypropy pseudouridine, 5-(isopentenylaminomethyl) uridine, 5-(isopentenylaminomethy])-2-thio-uridine, α-thio-uridine, 2′-O-methyl-uridine, 5,2′-O-dimethyl-uridine, 2′-O-methyl-pseudouridine, 2-thio-2′-O-methyl-uridine, 5-methoxycarbonylmethyl-2′-O-methyl-uridine, 5-carbamoylmethyl-2′-O-methyl-uridine, 5-carboxymethylaminomethyl-2′-O-methyl-uridine, 3,2′-O-dimethyl-uridine, 5-(isopentenylaminomethyl)-2′-O-methyl-uridine, l-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, pyrazolo[3,4-d]pyrimidines, xanthine, and hypoxanthine.

In some aspects, the chemical modification described herein comprises modifying a cytosine. In some aspects, the oligonucleotide described herein comprises at least one chemically modified cytosine. Exemplary chemically modified cytosine can include 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine, 5-hydroxymethyl-cytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine, a-thio-cytidine, 2′-O-methyl-cytidine, 5,2′-O-dimethyl-cytidine, N4-acetyl-2′-O-methyl-cytidine, N4,2′-O-dimethyl-cytidine, 5-formyl-2′-O-methyl-cytidine, N4,N4,2′-O-trimethyl-cytidine, 1-thio-cytidine, 2′-F-ara-cytidine, 2′-F-cytidine, and 2′-OH-ara-cytidine.

In some aspects, the chemical modification described herein comprises modifying a adenine. In some aspects, the oligonucleotide described herein comprises at least one chemically modified adenine. Exemplary chemically modified adenine can include 2-amino-purine, 2,6-diaminopurine, 2-amino-6-halo-purine (e.g., 2-amino-6-chloro-purine), 6-halo-purine (e.g., 6-chloi-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine, 2-methyl-adenine, N6-methyl-adenosine, 2-methylthio-N6-methyl-adenosine, N6-isopentenyl-adenosine, 2-methylthio-N6-isopentenyl-adenosine, N6-(cis-hydroxyisopentenyl) adenosine, 2-methylthio-N6-(cis-hydroxyisopentenyl) adenosine, N6-glycinylcarbamoyl-adenosine, N6-threonylcarbamoyl-adenosine, N6-methyl-N6-threonylcarbamoyl-adenosine, 2-methylthio-N6-threonylcarbamoyl-adenosine, N6, N6-dimethyladenosine, N6-hydroxynorvalylcarbamoyl-adenosine, 2-methylthio-N6-hydroxynorvalylcarbamoyl-adenosine, N6-acetyl-adenosine, 7-methyl-adenine, 2-methylthio-adenine, 2-methoxy-adenine, a-thio-adenosine, 2′-O-methyl-adenosine, N6, 2′—O-dimethyladenosine, N6-Methyl-2′-deoxyadenosine, N6, N6, 2′—O-trimethyl-adenosine, 1,2′-O-dimethyladenosine, 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some aspects, the chemical modification described herein comprises modifying a guanine. In some aspects, the oligonucleotide described herein comprises at least one chemically modified guanine. Exemplary chemically modified guanine can include inosine, 1-methyl-inosine, wyosine, methylwyosine, 4-demethyl-wyosine, isowyosine, wybutosine, peroxywybutosine, hydroxywybutosine, undermodified hydroxywybutosine, 7-deaza-guanosine, queuosine, epoxyqueuosine, galactosyl-queuosine, mannosyl-queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, 6-methoxy-guanosine, 1-methyl-guanosine, N2-methyl-guanosine, N2, N2-dimethyl-guanosine, N2, 7-dimethyl-guanosine, N2, N2, 7-dimethyl-guanosine, 8-oxo-guanosine, 7-methyl-8-oxo-guanosine, 1-meththio-guanosine, N2-methyl-6-thio-guanosine, N2,N2-dimethyl-6-thio-guanosine, a-thio-guanosine, 2′-O-methyl-guanosine, N2-methyl-2′-O-methyl-guanosine, N2,N2-dimethyl-2′-O-methyl-guanosine, 1-methyl-2′-O-methyl-guanosine, N2, 7-dimethyl-2′-O-methyl-guanosine, 2′-O-methyl-inosine, 1, 2′—O-dimethyl-inosine, 6-O-phenyl-2′-deoxyinosine, 2′-O-ribosylguanosine, 1-thio-guanosine, 6-O-methylguanosine, O⁶-Methyl-2′-deoxyguanosine, 2′-F-ara-guanosine, and 2′-F-guanosine.

In some cases, the chemical modification of the oligonucleotide can include introducing or substituting a nucleic acid analog or an unnatural nucleic acid into the oligonucleotide. In some aspects, nucleic acid analog can be any one of the chemically modified nucleic acid described herein. all of which are expressly incorporated by reference in their entireties. The chemically modified nucleotide described herein can include a variant of guanosine, uridine, adenosine, thymidine, and cytosine, including any natively occurring or non-natively occurring guanosine, uridine, adenosine, thymidine or cytidine that has been altered chemically, for example by acetylation, methylation, hydroxylation. Exemplary chemically modified nucleotide can include 1-methyl-adenosine, 1-methyl-guanosine, 1-methyl-inosine, 2,2-dimethyl-guanosine, 2,6-diaminopurine, 2′-amino-2′-deoxyadenosine, 2′-amino-2′-deoxycytidine, 2′-amino-2′-deoxyguanosine, 2′-amino-2′-deoxyuridine, 2-amino-6-chloropurineriboside, 2-aminopurine-riboside, 2′-araadenosine, 2′-aracytidine, 2′-arauridine, 2′-azido-2′-deoxyadenosine, 2′-azido-2′-deoxycytidine, 2′-azido-2′-deoxyguanosine, 2′-azido-2′-deoxyuridine, 2-chloroadenosine, 2′-fluoro-2′-deoxyadenosine, 2′-fluoro-2′-deoxycytidine, 2′-fluoro-2′-deoxyguanosine, 2′-fluoro-2′-deoxyuridine, 2′-fluorothymidine, 2-methyl-adenosine, 2-methyl-guanosine, 2-methyl-thio-N6-isopenenyl-adenosine, 2′-O-methyl-2-aminoadenosine, 2′-O-methyl-2′-deoxyadenosine, 2′-O-methyl-2′-deoxycytidine, 2′—O-methyl-2′-deoxyguanosine, 2, —O-methyl-2′-deoxyuridine, 2′-O-methyl-5-methyluridine, 2′-O-methylinosine, 2′-O-methylpseudouridine, 2-thiocytidine, 2-thio-cytidine, 3-methyl-cytidine, 4-acetyl-cytidine, 4-thiouridine, 5-(carboxyhydroxymethyl)-uridine, 5,6-dihydrouridine, 5-aminoallylcytidine, 5-aminoallyl-deoxyuridine, 5-bromouridine, 5-carboxymethylaminomethyl-2-thio-uracil, 5-carboxymethylamonomethyl-uracil, 5-chloro-ara-cytosine, 5-fluoro-uridine, 5-iodouridine, 5-methoxycarbonylmethyl-uridine, 5-methoxy-uridine, 5-methyl-2-thio-uridine, 6-Azacytidine, 6-azauridine, 6-chloro-7-deaza-guanosine, 6-chloropurineriboside, 6-mercapto-guanosine, 6-methyl-mercaptopurine-riboside, 7-deaza-2′-deoxy-guanosine, 7-deazaadenosine, 7-methyl-guanosine, 8-azaadenosine, 8-bromo-adenosine, 8-bromo-guanosine, 8-mercapto-guanosine, 8-oxoguanosine, benzimidazole-riboside, beta-D-mannosyl-queosine, dihydro-uridine, inosine, N1-methyladenosine, N6-([6-aminohexyl]carbamoylmethyl)-adenosine, N6-isopentenyl-adenosine, N6-methyl-adenosine, N7-methyl-xanthosine, N-uracil-5-oxyacetic acid methyl ester, puromycin, queosine, uracil-5-oxyacetic acid, uracil-5-oxyacetic acid methyl ester, wybutoxosine, xanthosine, and xylo-adenosine. In some aspects, the chemically modified nucleic acid as described herein comprises at least one chemically modified nucleotide selected from 2-amino-6-chloropurineriboside-5′-triphosphate, 2-aminopurine-riboside-5′-triphosphate, 2-aminoadenosine-5′-triphosphate, 2′-amino-2′-deoxycytidine-triphosphate, 2-thiocytidine-5′-triphosphate, 2-thiouridine-5′-triphosphate, 2′-fluorothymidine-5′-triphosphate, 2′-O-methyl-inosine-5′-triphosphate, 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-triphosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 6-methylguanosine-5′-triphosphate, pseudouridine-5′-triphosphate, puromycin-5′-triphosphate, or xanthosine-5′-triphosphate. In some aspects, the chemically modified nucleic acid as described herein comprises at least one chemically modified nucleotide selected from pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some aspects, the artificial nucleic acid as described herein comprises at least one chemically modified nucleotide selected from 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some aspects, the chemically modified nucleic acid as described herein comprises at least one chemically modified nucleotide selected from 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In other embodiments, the chemically modified nucleic acid as described herein comprises at least one chemically modified nucleotide selected from inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In certain embodiments, the chemically modified nucleic acid as described herein comprises at least one chemically modified nucleotide selected from 6-aza-cytidine, 2-thio-cytidine, alpha-thio-cytidine, pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxythymidine, 5-methyl-uridine, pyrrolo-cytidine, inosine, alpha-thio-guanosine, 6-methyl-guanosine, 5-methyl-cytidine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, alpha-thio-adenosine, 8-azido-adenosine, 7-deazaadenosine.

A modified base of a unnatural nucleic acid includes, but is not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, 0-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and 0-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C—CH₃) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one), those in which the purine or pyrimidine base is replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, or 2-amino-2′-deoxyadenosine.

In some cases, the at least one chemical modification comprises chemically modifying the 5′ or 3′ end such as 5′ cap or 3′ tail of the oligonucleotide. In some aspects, the oligonucleotide comprises a chemical modification comprising 3′ nucleotides which can be stabilized against degradation, e.g., by incorporating one or more of the modified nucleotides described herein. In this embodiment, uridines can be replaced with modified uridines, e.g., 5-(2-amino) propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein. In some aspects, deaza nucleotides, e.g., 7-deaza-adenosine, can be incorporated into the gRNA. In some aspects, O- and N-alkylated nucleotides, e.g., N6-methyladenosine, can be incorporated into the gRNA. In some aspects, sugar-modified ribonucleotides can be incorporated, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In some aspects, the phosphate backbone can be modified as described herein, e.g., with a phosphothioate group. In some aspects, the nucleotides in the overhang region of the gRNA can each independently be a modified or unmodified nucleotide including, but not limited to 2′-sugar modified, such as, 2-F 2′-O-methyl, thymidine (T), 2′-O-methoxyethyl-5-methyluridine (Teo), 2′-O-methoxyethyladenosine (Aeo), 2′-O-methoxyethyl-5-methylcytidine (m5Ceo), or any combinations thereof.

In some aspects, the oligonucleotide comprising at least one chemical modification, upon binding to the target RNA, is more specific in recruiting the endogenous nuclease for decreasing expression the target RNA compared to an oligonucleotide sharing identical nucleic acid sequence, but without any chemical modification, with the oligonucleotide comprising at least one chemical modification. In some aspects, the oligonucleotide comprising at least one chemical modification is at least 10%, 20%, 30%, 40%, 50%, 60%, 70%, 80%, 90%, two fold, three fold, four fold, five fold, six fold, seven fold, eight fold, nine fold, 10 fold, 20 fold, 30 fold, 40 fold, 50 fold, 100 fold, 500 fold, 1000 fold, or more specific in recruiting the endogenous nuclease for decreasing expression the target RNA compared to an oligonucleotide sharing identical nucleic acid sequence, but without any chemical modification, with the oligonucleotide comprising at least one chemical modification.

In some aspects, the oligonucleotide comprising at least one chemical modification comprises an increased resistance towards degradation by hydrolysis compared to an oligonucleotide sharing identical nucleic acid sequence, but without any chemical modification, with the oligonucleotide comprising at least one chemical modification. In some aspects, the oligonucleotide comprising the at least one chemical modification is at least 10%, 20%, 30%, 40%, 50%, 60%, 70%, 80%, 90%, two fold, three fold, four fold, five fold, six fold, seven fold, eight fold, nine fold, 10 fold, 20 fold, 30 fold, 40 fold, 50 fold, 100 fold, 500 fold, 1000 fold, or more resistant towards degradation by hydrolysis compared to an oligonucleotide sharing identical nucleic acid sequence, but without any chemical modification, with the oligonucleotide comprising at least one chemical modification.

In some aspects, the oligonucleotide comprising at least one chemical modification comprises an increased resistance towards degradation by nuclease digestion compared to an oligonucleotide sharing identical nucleic acid sequence, but without any chemical modification, with the oligonucleotide comprising at least one chemical modification. In some aspects, the oligonucleotide comprising the at least one chemical modification is at least 10%, 20%, 30%, 40%, 50%, 60%, 70%, 80%, 90%, two fold, three fold, four fold, five fold, six fold, seven fold, eight fold, nine fold, 10 fold, 20 fold, 30 fold, 40 fold, 50 fold, 100 fold, 500 fold, 1000 fold, or more resistant towards degradation by nuclease digestion compared to an oligonucleotide sharing identical nucleic acid sequence, but without any chemical modification, with the oligonucleotide comprising at least one chemical modification.

In some aspects, the oligonucleotide comprising at least one chemical modification induces less immunogenicity compared an oligonucleotide sharing identical nucleic acid sequence, but without any chemical modification, with the oligonucleotide comprising at least one chemical modification. In some aspects, the oligonucleotide comprising the at least chemical modification is at least 10%, 20%, 30%, 40%, 50%, 60%, 70%, 80%, 90%, two fold, three fold, four fold, five fold, six fold, seven fold, eight fold, nine fold, 10 fold, 20 fold, 30 fold, 40 fold, 50 fold, 100 fold, 500 fold, 1000 fold, or more less likely to induce immunogenicity compared to immunogenicity induced by an oligonucleotide sharing identical nucleic acid sequence, but without any chemical modification, with the oligonucleotide comprising at least one chemical modification.

In some aspects, the oligonucleotide comprising at least one chemical modification induces less innate immune response relative to an oligonucleotide sharing identical nucleic acid sequence, but without any chemical modification, with the oligonucleotide comprising at least one chemical modification. In some aspects, the oligonucleotide comprising the at least one chemical modification is at least 10%, 20%, 30%, 40%, 50%, 60%, 70%, 80%, 90%, two fold, three fold, four fold, five fold, six fold, seven fold, eight fold, nine fold, 10 fold, 20 fold, 30 fold, 40 fold, 50 fold, 100 fold, 500 fold, 1000 fold, or more less likely to induce innate immune response compared to innate immune response induced by an oligonucleotide sharing identical nucleic acid sequence, but without any chemical modification, with the oligonucleotide comprising at least one chemical modification.

In some aspects, the oligonucleotide comprising at least one chemical modification, when contacted with the target RNA, is less likely to induce off-target modulating of the target RNA compared to the off-target modulating of the target RNA induced by an oligonucleotide sharing identical nucleic acid sequence, but without any chemical modification, with the oligonucleotide comprising at least one chemical modification. In some aspects, the oligonucleotide comprising the at least one chemical modification is at least 10%, 20%, 30%, 40%, 50%, 60%, 70%, 80%, 90%, two fold, three fold, four fold, five fold, six fold, seven fold, eight fold, nine fold, 10 fold, 20 fold, 30 fold, 40 fold, 50 fold, 100 fold, 500 fold, 1000 fold, or more less likely to induce off-target modulating compared to off-target modulating induced by an oligonucleotide sharing identical nucleic acid sequence, but without any chemical modification, with the oligonucleotide comprising at least one chemical modification.

### Methods of Delivery

Described herein, In some aspects, are methods of delivering the oligonucleotide described herein to a cell. In some aspects, the method comprises delivering directly or indirectly an oligonucleotide to the cell. In some aspects, the method comprises contacting the cell with the composition or the oligonucleotide described herein. In some aspects, the method comprises expressing the composition or the oligonucleotide described herein in the cell. In some aspects, the oligonucleotide or vector encoding the oligonucleotide can be delivered into the cell via any of the transfection methods described herein. In some aspects, the oligonucleotide can be delivered into the cell via the use of expression vectors. In the context of an expression vector, the vector can be readily introduced into the cell described herein by any method in the art. For example, the expression vector can be transferred into the cell by physical, chemical, or biological means.

Physical methods for introducing the oligonucleotide or vector encoding the oligonucleotide into the cell can include calcium phosphate precipitation, lipofection, particle bombardment, microinjection, gene gun, electroporation, and the like. Methods for producing cells comprising vectors and/or exogenous nucleic acids are suitable for methods herein. One method for the introduction of oligonucleotide or vector encoding the oligonucleotide into a host cell is calcium phosphate transfection.

Chemical means for introducing the oligonucleotide or vector encoding the oligonucleotide into the cell can include colloidal dispersion systems, such as macromolecule complexes, nanocapsules, microspheres, beads, and lipid-based systems including oil-in-water emulsions, micelles, mixed micelles, spherical nucleic acid (SNA), liposomes, or lipid nanoparticles. An exemplary colloidal system for use as a delivery vehicle in vitro and in vivo is a liposome (e.g., an artificial membrane vesicle). Other methods of state-of-the-art targeted delivery of nucleic acids are available, such as delivery of oligonucleotide or vector encoding the oligonucleotide with targeted nanoparticles or other suitable sub-micron sized delivery system.

In the case where a non-viral delivery system is utilized, an exemplary delivery vehicle is a liposome. The use of lipid formulations is contemplated for the introduction of the oligonucleotide or vector encoding the oligonucleotide into a cell (in vitro, ex vivo or in vivo). In another aspect, the oligonucleotide or vector encoding the oligonucleotide can be associated with a lipid. The oligonucleotide or vector encoding the oligonucleotide associated with a lipid, In some aspects, is encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, In some aspects, they are present in a bilayer structure, as micelles, or with a “collapsed” structure. Alternately, they are simply be interspersed in a solution, possibly forming aggregates that are not uniform in size or shape. Lipids are fatty substances which are, In some aspects, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use are obtained from commercial sources. Stock solutions of lipids in chloroform or chloroform/methanol are often stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes are often characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, compositions that have different structures in solution than the normal vesicular structure are also encompassed. For example, the lipids, In some aspects, assume a micellar structure or merely exist as nonuniform aggregates of lipid molecules. Also contemplated are lipofectamine-nucleic acid complexes.

In some cases, non-viral delivery method comprises lipofection, nucleofection, microinjection, biolistics, virosomes, liposomes, immunoliposomes, exosomes, polycation or lipid:cargo conjugates (or aggregates), naked polypeptide (e.g., recombinant polypeptides), naked DNA, artificial virions, and agent-enhanced uptake of polypeptide or DNA. In some aspects, the delivery method comprises conjugating or encapsulating the compositions or the oligonucleotides described herein with at least one polymer such as natural polymer or synthetic materials. The polymer can be biocompatible or biodegradable. Non-limiting examples of suitable biocompatible, biodegradable synthetic polymers can include aliphatic polyesters, poly(amino acids), copoly(ether-esters), polyalkylenes oxalates, polyamides, poly(iminocarbonates), polyorthoesters, polyoxaesters, polyamidoesters, polyoxaesters containing amine groups, and poly(anhydrides). Such synthetic polymers can be homopolymers or copolymers (e.g., random, block, segmented, graft) of a plurality of different monomers, e.g., two or more of lactic acid, lactide, glycolic acid, glycolide, epsilon-caprolactone, trimethylene carbonate, p-dioxanone, etc. In an example, the scaffold can be comprised of a polymer comprising glycolic acid and lactic acid, such as those with a ratio of glycolic acid to lactic acid of 90/10 or 5/95. Non-limiting examples of naturally occurring biocompatible, biodegradable polymers can include glycoproteins, proteoglycans, polysaccharides, glycosamineoglycan (GAG) and fragment(s) derived from these components, elastin, laminins, decrorin, fibrinogen/fibrin, fibronectins, osteopontin, tenascins, hyaluronic acid, collagen, chondroitin sulfate, heparin, heparan sulfate, ORC, carboxymethyl cellulose, and chitin.

In some cases, the oligonucleotide or vector encoding the oligonucleotide described herein can be packaged and delivered to the cell via extracellular vesicles. The extracellular vesicles can be any membrane-bound particles. In some aspects, the extracellular vesicles can be any membrane-bound particles secreted by at least one cell. In some instances, the extracellular vesicles can be any membrane-bound particles synthesized in vitro. In some instances, the extracellular vesicles can be any membrane-bound particles synthesized without a cell. In some cases, the extracellular vesicles can be exosomes, microvesicles, retrovirus-like particles, apoptotic bodies, apoptosomes, oncosomes, exophers, enveloped viruses, exomeres, or other very large extracellular vesicles.

In some cases, the oligonucleotide or vector encoding the oligonucleotide described herein can be administered to the subject in need thereof via the use of the transgenic cells generated by introduction of the oligonucleotide or vector encoding the oligonucleotide first into allogeneic or autologous cells. In some cases, the cell can be isolated. In some aspects, the cell can be isolated from the subject.

In some aspects, the oligonucleotide described herein is conjugated. In some aspects, the oligonucleotide is conjugated to with a peptide, antibody, lipid, carbohydrate, or polymer. In some aspects, the oligonucleotide is conjugated to with a peptide, antibody, lipid, carbohydrate, or polymer at the 5′ end of the oligonucleotide. In some aspects, the oligonucleotide is conjugated to with a peptide, antibody, lipid, carbohydrate, or polymer at the 3′ end of the oligonucleotide. In some aspects, the oligonucleotide is conjugated to with a peptide, antibody, lipid, carbohydrate, or polymer at any nucleic acid residue of the oligonucleotide. In some aspects, the peptide, antibody, lipid, carbohydrate, or polymer conjugated to the oligonucleotide confers therapeutic effect. For example, the peptide, antibody, lipid, carbohydrate, or polymer conjugated to the oligonucleotide can be cytotoxic drug or drug for treating cancer. In some aspects, the peptide, antibody, lipid, carbohydrate, or polymer conjugated to the oligonucleotide increases the efficiency of the oligonucleotide binding to the endogenous nucleic acid. In some aspects, the peptide, antibody, lipid, carbohydrate, or polymer conjugated to the oligonucleotide confers targeting specificity of the oligonucleotide to specific types of cells (e.g., cancer cells, etc.). In some aspects, the peptide, antibody, lipid, carbohydrate, or polymer conjugated to the oligonucleotide confers stability of the oligonucleotide in vitro, ex vivo, or in vivo. For example, the oligonucleotide can be conjugated with polyethylene glycol (PEG) or endosomolytic agent to decrease immunogenicity or degradation. In some aspects, the peptide, antibody, lipid, carbohydrate, or polymer conjugated to the oligonucleotide to facilitate the oligonucleotide for entering cell. In some aspects, the peptide, antibody, lipid, carbohydrate, or polymer conjugated to the oligonucleotide to facilitate and release to the oligonucleotide in the cell. In some aspects, the peptide, antibody, lipid, carbohydrate, or polymer conjugated to the oligonucleotide comprises at least one targeting moiety for targeting the cell. Non-limiting examples of the targeting moiety comprises a signaling peptide, a chemokine, a chemokine receptor, an adhesion molecule, an antigen, or an antibody.

The linker for conjugating the oligonucleotide to the peptide, antibody, lipid, or polymer can be any linker that connects biomolecules. In some aspects, a linker described herein is a cleavable linker or a non-cleavable linker. In some instances, the linker is a cleavable linker. In other instances, the linker is a non-cleavable linker. In some cases, the linker is a non-polymeric linker. A non-polymeric linker refers to a linker that does not contain a repeating unit of monomers generated by a polymerization process. In some aspects, the linker comprises a peptide moiety. In some instances, the peptide moiety comprises at least 2, 3, 4, 5, or 6 more amino acid residues. In some aspects, the linker comprises a benzoic acid group, or its derivatives thereof. In some aspects, the linker can comprise nucleic acid linker such as DNA linker. In such case, the peptide, antibody, lipid, or polymer can be conjugated on one end of the nucleic acid linker or intercalated into the nucleic acid base pairing of the nucleic acid linker. In some aspects, the linker can be a peptide linker. The peptide linker can be flexible (e.g., poly-glycine linker) or rigid (e.g., EAAAK repeat linker). In some aspects, the peptide linker can be cleaved (e.g., a disulfide bond). In some aspects, the linker comprises polymers such PEG, polylactic acid (PLA), or polyacrylic acid (PAA).

### Methods of Treatment

Disclosed herein, in some aspects, are methods of modulating KRAS-mediated signaling pathway in a cancer cell by treating or contacting the cancer cell with a composition comprising antisense oligonucleotide, composition, or pharmaceutical composition described herein, thereby reducing expression of KRAS or mutated KRAS protein or mRNA in the cancer cell. In some embodiments, mutated KRAS protein comprising a G12C mutation, a G12V mutation, a G12A mutation, or a G12D mutation.

Also disclosed herein, in some aspects, are methods of treating a subject in need thereof by administrating a therapeutic effective amount of the oligonucleotide, composition, or pharmaceutical composition described herein to the subject. In some aspects, the method treats the subject by modulating gene expression or signaling pathway expression in the subject. In some aspects, the method comprises decreasing gene expression by contacting endogenous nucleic acid (e.g. endogenous mRNA) with the oligonucleotide described herein. In some aspects, the method comprises decreasing KRAS, mutated KRAS, or a combination of KRAS and mutated KRAS in the subject or in the cancer cell by contacting mRNA of KRAS or mutated KRAS with the oligonucleotide described herein, where the binding of the oligonucleotide to the mRNA recruits endogenous nuclease for degradation of the mRNA. In some aspects, the method comprises decreasing expression of signaling pathway such as KRAS-mediated signaling pathway. In some aspects, the method comprises decreasing expression of a gene in or the activity of the KRAS-RAF-MEK-ERK signaling pathway, the PI3K signaling pathway, the MAPK signaling pathway, or the Ral-GEF signaling pathway.

In some aspects, the oligonucleotide, composition, or pharmaceutical composition can be administered to the subject alone (e.g., standalone treatment). In some aspects, the oligonucleotide, composition, or pharmaceutical composition is administered in combination with an additional agent. In some cases, the additional agent as used herein is administered alone. The oligonucleotide, composition, or pharmaceutical composition and the additional agent can be administered together or sequentially. Non-limiting examples of the additional agent comprise N-(2-(4-(4-bis(2-chloroethyl)aminophenyl)butyryl)aminoethyl)-5-(4-amidinophenyl)-2-furanecarboxamide hydrochloride; Allyl isothiocyanate; Benzyl isothiocyanate; Phenethyl isothiocyanate; Belinostat; Berberin; Casticin; Chrysin; Bufalin; Fisetin; Fucoidan; Galic acid; Gemcitabine; Guizhi Fuling Decoction; JOTO1007; Quercetin; Rasfonin; 2,3,7,8-tetrachlorodibenzodioxin; Triptolide; 4-Hydroxybutenolide; or a combination thereof. The combination therapies can be administered within the same day, or can be administered one or more days, weeks, months, or years apart.

In some aspects, the oligonucleotide, composition, or pharmaceutical composition is a first-line treatment for the disease or condition. In some aspects, the oligonucleotide, composition, or pharmaceutical composition is a second-line, third-line, or fourth-line treatment. In some aspects, the oligonucleotide, composition, or pharmaceutical composition comprises at least one, two, three, four, five, six, seven, eight, nine, 10, 20, 30 or more oligonucleotide. In general, method disclosed herein comprises administering the oligonucleotide, composition, or pharmaceutical composition by oral administration. However, in some instances, method comprises administering the oligonucleotide, composition, or pharmaceutical composition by intraperitoneal injection. In some instances, the method comprises administering the pharmaceutical composition in the form of an anal suppository. In some instances, the method comprises administering the oligonucleotide, composition, or pharmaceutical composition by intravenous (“i.v.”) administration. It is conceivable that one can also administer the oligonucleotide, composition, or pharmaceutical composition disclosed herein by other routes, such as subcutaneous injection, intramuscular injection, intradermal injection, transdermal injection percutaneous administration, intranasal administration, intralymphatic injection, rectal administration intragastric administration, or any other suitable parenteral administration. In some aspects, routes for local delivery closer to site of injury or inflammation are preferred over systemic routes. Routes, dosage, time points, and duration of administrating therapeutics can be adjusted. In some aspects, administration of therapeutics is prior to, or after, onset of either, or both, acute and chronic symptoms of the disease or condition.

Suitable dose and dosage administrated to a subject is determined by factors including, but no limited to, the particular the oligonucleotide, composition, or pharmaceutical composition, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject being treated.

In some aspects, the administration of the oligonucleotide, composition, or pharmaceutical composition described herein is hourly, once every 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours 22 hours, 23 hours, 1 day, 2 days, 3 days, 4 days, 5 days, 6 days, 7 days, 8 days, 9 days, 10 days, 11 days, 12 days, 13 days, 14 days, 15 days, 1 month, 2 months, 3 months, 4 months, 5 months, 6 months, 7 months, 8 months, 9 months, 10 months, 11 months, 1 year, 2 years, 3 years, 4 years, or 5 years, or 10 years. The effective dosage ranges can be adjusted based on subject's response to the treatment. Some routes of administration will require higher concentrations of effective amount of therapeutics than other routes.

In some aspects, the administration of the oligonucleotide, composition, or pharmaceutical composition described herein increases survival rate of the subject by at least 1%, 2%, 3%, 4%, 5%, 6%, 7%, 8%, 9%, 10%, 15%, 20%, 30%, 40%, 50%, or more. In some aspects, the administration of the oligonucleotide, composition, or pharmaceutical composition described herein at a dose that increases survival rate of the subject by at least 1%, 2%, 3%, 4%, 5%, 6%, 7%, 8%, 9%, 10%, 15%, 20%, 30%, 40%, 50%, or more. In some aspects, the administration of the oligonucleotide, composition, or pharmaceutical composition described herein at a schedule that increases survival rate of the subject by at least 1%, 2%, 3%, 4%, 5%, 6%, 7%, 8%, 9%, 10%, 15%, 20%, 30%, 40%, 50%, or more. In some aspects, the administration of the oligonucleotide, composition, or pharmaceutical composition described herein at a dose and a schedule that increase survival rate of the subject by at least 1%, 2%, 3%, 4%, 5%, 6%, 7%, 8%, 9%, 10%, 15%, 20%, 30%, 40%, 50%, or more.

In some aspects, the administration of the oligonucleotide, composition, or pharmaceutical composition described herein inhibits growth of the tumor by at least 1%, 2%, 3%, 4%, 5%, 6%, 7%, 8%, 9%, 10, 1%, 20%, 30%, 40%, 50%, or more. In some aspects, the administration of the oligonucleotide, composition, or pharmaceutical composition described herein at a dose that inhibits growth of the tumor by at least 1%, 2%, 3%, 4%, 5%, 6%, 7%, 8%, 9%, 10%, 15%, 20%, 30%, 40%, 50%, or more. In some aspects, the administration of the oligonucleotide, composition, or pharmaceutical composition described herein at a schedule that inhibits growth of the tumor by at least 1%, 2%, 3%, 4%, 5%, 6%, 7%, 8%, 9%, 10%, 15%, 20%, 30%, 40%, 50%, or more. In some aspects, the administration of the oligonucleotide, composition, or pharmaceutical composition described herein at a dose and a schedule that inhibits growth of the tumor by at least 1%, 2%, 3%, 4%, 5%, 6%, 7%, 8%, 9%, 10%, 15%, 20%, 30%, 40%, 50%, or more.

In some aspects, the administration of the oligonucleotide, composition, or pharmaceutical composition described herein to the subject in a dose that is sufficient to inhibit growth of the tumor. In some aspects, the administration of the oligonucleotide, composition, or pharmaceutical composition described herein to the subject in a schedule that is sufficient to inhibit growth of the tumor. In some aspects, the administration of the oligonucleotide, composition, or pharmaceutical composition described herein to the subject in a dose and a schedule that are sufficient to inhibit growth of the tumor.

In certain embodiments, where the subject's condition does not improve, upon the doctor's discretion the administration of the pharmaceutical composition is administered chronically, that is, for an extended period of time, including throughout the duration of the subject's life in order to ameliorate or otherwise control or limit the symptoms of the subject's disease or condition. In certain embodiments wherein a subject's status does improve, the dose of the pharmaceutical composition being administered can be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. In certain embodiments, the dose of the pharmaceutical composition being administered can be temporarily reduced or temporarily suspended for a certain length of time (i.e., a “drug diversion”). In specific embodiments, the length of the pharmaceutical composition diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during the pharmaceutical composition diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

In some aspects, once improvement of the subject's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the subject requires intermittent treatment on a long-term basis upon any recurrence of symptoms.

Toxicity and therapeutic efficacy of such therapeutic regimens are determined by standard pharmaceutical procedures in cell cultures or experimental animals, including, but not limited to, the determination of the LD50 and the ED50. The dose ratio between the toxic and therapeutic effects is the therapeutic index and it is expressed as the ratio between LD50 and ED50. In certain embodiments, the data obtained from cell culture assays and animal studies are used in formulating the therapeutically effective daily dosage range and/or the therapeutically effective unit dosage amount for use in mammals, including humans. In some aspects, the daily dosage amount of the composition described herein lies within a range of circulating concentrations that include the ED50 with minimal toxicity. In certain embodiments, the daily dosage range and/or the unit dosage amount varies within this range depending upon the dosage form employed and the route of administration utilized.

In some aspects, the disease or condition described herein is a cancer. In some aspects, the cancer is associated with KRAS. In some aspects, the cancer is associated with MUTATED KRAS. In some aspects, the cancer is associated with KRAS. In some aspects, the cancer is associated with an abnormality of KRAS-mediated signaling pathway. In some aspects, the cancer is a lung cancer, a pancreatic cancer, or a colon cancer. Other non-limiting examples of the cancer can include Acute Lymphoblastic Leukemia, Acute Lymphocytic Leukemia (ALL), Acute Myeloid Leukemia (AML), Adenoid Cystic Carcinoma, Adrenal Gland Cancer, Adrenocortical Carcinoma, Adult Leukemia, AIDS-Related Lymphoma, Amyloidosis, Anal Cancer, Astrocytomas, Ataxia Telangiectasia, Atypical Mole Syndrome, Atypical Teratoid/Rhabdoid Tumor, Basal Cell Carcinoma, Bile Duct Cancer, Birt Hogg Dube Syndrome, Bladder Cancer, Bone Cancer, Brain Tumor, Breast Cancer, Bronchial Tumors, Burkitt Lymphoma, Carcinoid Tumor (Gastrointestinal), Carcinoma of Unknown Primary, Cardiac (Heart) Tumors, Cervical Cancer, Cholangiocarcinoma, Chordoma, Chronic Lymphocytic Leukemia (CLL), Chronic Myelogenous Leukemia, Chronic Myeloid Leukemia, Chronic Myeloproliferative Neoplasms, Colorectal Cancer, Craniopharyngioma, Cutaneous T-Cell Lymphoma, Ductal Carcinoma, Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Eye Cancer, Fallopian Tube Cancer, Fibrous Histiocytoma of Bone, Malignant, and Osteosarcoma, Gallbladder Cancer, Gastric Cancer, Gastrointestinal Carcinoid Tumor, Gastrointestinal Stromal Tumor (GIST), Germ Cell Tumors, Gestational Trophoblastic Disease, Hairy Cell Leukemia, Head and Neck Cancer, Hepatocellular Cancer, HER2-Positive Breast Cancer, Histiocytosis, Langerhans Cell, Hodgkin's Lymphoma, Hypopharyngeal Cancer, Intraocular Melanoma, Islet Cell Tumor, Juvenile Polyposis Syndrome, Kaposi Sarcoma, Kidney Cancer, Langerhans Cell Histiocytosis, Laryngeal Cancer, Leukemia, Lip and Oral Cavity Cancer, Liver Cancer, Lobular Carcinoma, Lung Cancer (Non-Small Cell and Small Cell), Lymphoma, Malignant Fibrous Histiocytoma of Bone and Osteosarcoma, Malignant Glioma, Melanoma, Intraocular Melanoma, Meningioma, Merkel Cell Carcinoma, Mesothelioma, Malignant, Metastatic Cancer, Metastatic Squamous Neck Cancer with Occult Primary, Midline Tract Carcinoma, Multiple Endocrine Neoplasia Syndromes, Multiple Myeloma, Plasma Cell Neoplasms, Mycosis Fungoides, Myelodysplastic Syndrome (MDS), Myeloproliferative Neoplasms, Chronic, Nasal Cavity and Paranasal Sinus Cancer, Nasopharyngeal Cancer, Neuroblastoma, Neuroendocrine Tumor, Non-Hodgkin Lymphoma, Oral Cancer, Lip and Oral Cavity Cancer and Oropharyngeal Cancer, Oropharyngeal Cancer, Osteosarcoma, Ovarian Cancer, Ovarian Germ Cell Tumors, Pancreatic Cancer, Pancreatic Neuroendocrine Tumors, Papillomatosis, Paraganglioma, Paranasal Sinus and Nasal Cavity Cancer, Parathyroid Cancer, Penile Cancer, Peritoneal Cancer, Peutz-Jeghers Syndrome, Pharyngeal Cancer, Pheochromocytoma, Pituitary Tumor, Plasma Cell Neoplasm/Multiple Myeloma, Pleuropulmonary Blastoma, Polycythemia Vera, Pregnancy and Breast Cancer, Primary Central Nervous System (CNS) Lymphoma, Primary Peritoneal Cancer, Prostate Cancer, Rectal Cancer, Recurrent Cancer, Renal Cell Carcinoma, Retinoblastoma, Rhabdomyosarcoma, Salivary Gland Cancer, Sarcoma, Sezary Syndrome, Skin Cancer, Small Intestine Cancer, Soft Tissue Sarcoma, Solid tumor, Squamous Cell Carcinoma of the Skin, Squamous Neck Cancer with Occult Primary, Metastatic, Stomach Cancer, T-Cell Lymphoma, Testicular Cancer, Throat Cancer, Thymoma, Thymic Carcinoma, Thyroid Cancer, Transitional Cell Cancer of the Renal Pelvis and Ureter, Unusual Cancers of Childhood, Ureter and Renal Pelvis, Transitional Cell Cancer, Urethral Cancer, Uterine (Endometrial) Cancer, Uterine Sarcoma, Vaginal Cancer, Vascular Tumors, Vulvar Cancer, Wilms Tumor, or a combination thereof.

### Pharmaceutical Compositions

Described herein, in some aspects, is a pharmaceutical composition comprising the oligonucleotide or the composition described herein. Pharmaceutical composition, as used herein, refers to a mixture of a pharmaceutical composition, with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. Optionally, the compositions include two or more pharmaceutical composition as discussed herein. In practicing the methods of treatment or use provided herein, therapeutically effective amounts of pharmaceutical compositions described herein are administered in a pharmaceutical composition to a mammal having a disease, disorder, or condition to be treated, e.g., an inflammatory disease, fibrostenotic disease, and/or fibrotic disease. In some aspects, the mammal is a human. A therapeutically effective amount can vary widely depending on the severity of the disease, the age and relative health of the subject, the potency of the pharmaceutical composition used and other factors. The pharmaceutical compositions can be used singly or in combination with one or more pharmaceutical compositions as components of mixtures. The pharmaceutical commotions described herein comprise the oligonucleotide, the compositions, the cells contacted with the oligonucleotide or contacted with the composition comprising the oligonucleotide, or a combination thereof.

The pharmaceutical formulations described herein are administered to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a pharmaceutical composition are manufactured in a conventional manner, such as, by way of example only, by means of conventional mixing, dissolving, granulating, dragee-making, levigating, emulsifying, encapsulating, entrapping or compression processes.

The pharmaceutical compositions may include at least a pharmaceutical composition as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some aspects, pharmaceutical compositions exist in unsolvated form or in solvated forms with pharmaceutically acceptable solvents such as water, ethanol, and the like. The solvated forms of the pharmaceutical compositions are also considered to be disclosed herein.

In some aspects, a pharmaceutical composition exists as a tautomer. All tautomers are included within the scope of the agents presented herein. As such, it is to be understood that a pharmaceutical composition or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they can be regarded as different isomeric forms of the same compound.

In some aspects, a pharmaceutical composition exists as an enantiomer, diastereomer, or other steroisomeric form. The agents disclosed herein include all enantiomeric, diastereomeric, and epimeric forms as well as mixtures thereof.

In some aspects, pharmaceutical compositions described herein can be prepared as prodrugs. A “prodrug” refers to an agent that is converted into the parent drug in vivo. Prodrugs are often useful because, in some situations, they can be easier to administer than the parent drug. They may, for instance, be bioavailable by oral administration whereas the parent is not. The prodrug may also have improved solubility in pharmaceutical compositions over the parent drug. An example, without limitation, of a prodrug would be a pharmaceutical composition described herein, which is administered as an ester (the “prodrug”) to facilitate transmittal across a cell membrane where water solubility is detrimental to mobility but which then is metabolically hydrolyzed to the carboxylic acid, the active enzyme, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the pharmaceutical composition. In certain embodiments, a prodrug is enzymatically metabolized by one or more steps or processes to the biologically, pharmaceutically or therapeutically active form of the pharmaceutical composition.

Prodrug forms of the pharmaceutical compositions, wherein the prodrug is metabolized in vivo to produce an agent as set forth herein are included within the scope of the claims. Prodrug forms of the herein described pharmaceutical compositions, wherein the prodrug is metabolized in vivo to produce an agent as set forth herein are included within the scope of the claims. In some cases, some of the pharmaceutical compositions described herein can be a prodrug for another derivative or active compound. In some embodiments described herein, hydrazones are metabolized in vivo to produce a pharmaceutical composition.

### Kits

Described herein, In some aspects, are kits for using the oligonucleotide, the compositions, or the pharmaceutical compositions described herein. In some aspects, the kits disclosed herein may be used to treat a disease or condition in a subject. In some aspects, the kit comprises an assemblage of materials or components apart from the oligonucleotide, the composition, or the pharmaceutical composition. In some aspects, the kit comprises the components for assaying and selecting for suitable oligonucleotide for treating a disease or a condition. In some aspects, the kit comprises components for performing assays such as enzyme-linked immunosorbent assay (ELISA), single-molecular array (Simoa), PCR, or qPCR. The exact nature of the components configured in the kit depends on its intended purpose. For example, some embodiments are configured for the purpose of treating a disease or condition disclosed herein (e.g., cancer) in a subject. In some aspects, the kit is configured particularly for the purpose of treating mammalian subjects. In some aspects, the kit is configured particularly for the purpose of treating human subjects.

Instructions for use may be included in the kit. In some aspects, the kit comprises instructions for administering the composition to a subject in need thereof. In some aspects, the kit comprises instructions for further engineering the oligonucleotide. In some aspects, the kit comprises instructions thawing or otherwise restoring biological activity of the oligonucleotide, which may have been cryopreserved or lyophilized during storage or transportation. In some aspects, the kit comprises instructions for measuring efficacy for its intended purpose (e.g., therapeutic efficacy if used for treating a subject).

Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit may be provided to the practitioner stored in any convenient and suitable ways that preserve their operability and utility. For example the oligonucleotide, the composition, or the pharmaceutical composition may be in dissolved, dehydrated, or lyophilized form. The components are typically contained in suitable packaging material(s).

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

TABLE 7
Exemplary KRAS antisense oligonucleotide

| | Nucleic acid | | Nucleic acid |
|---|---|---|---|
| SEQ ID NO: | sequence | SEQ ID NO: | sequence |
| SEQ ID NO: 100 | TCCTCTATTGTTGG | SEQ ID NO: 328 | ACTAGGACCATAGG |
| SEQ ID NO: 101 | ATCCTCTATTGTTG | SEQ ID NO: 329 | TACTAGGACCATAG |
| SEQ ID NO: 102 | AATCCTCTATTGTT | SEQ ID NO: 330 | CTACTAGGACCATA |
| SEQ ID NO: 103 | GAATCCTCTATTGT | SEQ ID NO: 331 | CCTACTAGGACCAT |
| SEQ ID NO: 104 | GGAATCCTCTATTG | SEQ ID NO: 332 | TCCTACTAGGACCA |
| SEQ ID NO: 105 | AGGAATCCTCTATT | SEQ ID NO: 333 | TTCCTACTAGGACC |
| SEQ ID NO: 106 | TAGGAATCCTCTAT | SEQ ID NO: 334 | TTTCCTACTAGGAC |
| SEQ ID NO: 107 | GTAGGAATCCTCTA | SEQ ID NO: 335 | ATTTCCTACTAGGA |
| SEQ ID NO: 108 | TGTAGGAATCCTCT | SEQ ID NO: 336 | TATTTCCTACTAGG |
| SEQ ID NO: 109 | CTGTAGGAATCCTC | SEQ ID NO: 337 | TTATTTCCTACTAG |
| SEQ ID NO: 110 | CCTGTAGGAATCCT | SEQ ID NO: 338 | TTTATTTCCTACTA |
| SEQ ID NO: 111 | TCCTGTAGGAATCC | SEQ ID NO: 339 | ATTTATTTCCTACT |
| SEQ ID NO: 112 | TTCCTGTAGGAATC | SEQ ID NO: 340 | CATTTATTTCCTAC |
| SEQ ID NO: 113 | CTTCCTGTAGGAAT | SEQ ID NO: 341 | ACATTTATTTCCTA |
| SEQ ID NO: 114 | GCTTCCTGTAGGAA | SEQ ID NO: 342 | CACATTTATTTCCT |
| SEQ ID NO: 115 | TGCTTCCTGTAGGA | SEQ ID NO: 343 | TCACATTTATTTCC |
| SEQ ID NO: 116 | TTGCTTCCTGTAGG | SEQ ID NO: 344 | ATCACATTTATTTC |
| SEQ ID NO: 117 | CTTGCTTCCTGTAG | SEQ ID NO: 345 | AATCACATTTATTT |
| SEQ ID NO: 118 | ACTTGCTTCCTGTA | SEQ ID NO: 346 | AAATCACATTTATT |
| SEQ ID NO: 119 | TACTTGCTTCCTGT | SEQ ID NO: 347 | CAAATCACATTTAT |
| SEQ ID NO: 120 | CTACTTGCTTCCTG | SEQ ID NO: 348 | GCAAATCACATTTA |
| SEQ ID NO: 121 | ACTACTTGCTTCCT | SEQ ID NO: 349 | GGCAAATCACATTT |
| SEQ ID NO: 122 | TACTACTTGCTTCC | SEQ ID NO: 350 | AGGCAAATCACATT |
| SEQ ID NO: 123 | TTACTACTTGCTTC | SEQ ID NO: 351 | AAGGCAAATCACAT |
| SEQ ID NO: 124 | ATTACTACTTGCTT | SEQ ID NO: 352 | GAAGGCAAATCACA |
| SEQ ID NO: 125 | AATTACTACTTGCT | SEQ ID NO: 353 | AGAAGGCAAATCAC |
| SEQ ID NO: 126 | CAATTACTACTTGC | SEQ ID NO: 354 | TAGAAGGCAAATCA |
| SEQ ID NO: 127 | TCAATTACTACTTG | SEQ ID NO: 355 | CTAGAAGGCAAATC |
| SEQ ID NO: 128 | ATCAATTACTACTT | SEQ ID NO: 356 | TCTAGAAGGCAAAT |
| SEQ ID NO: 129 | CATCAATTACTACT | SEQ ID NO: 357 | TTCTAGAAGGCAAA |
| SEQ ID NO: 130 | CCATCAATTACTAC | SEQ ID NO: 358 | GTTCTAGAAGGCAA |
| SEQ ID NO: 131 | TCCATCAATTACTA | SEQ ID NO: 359 | TGTTCTAGAAGGCA |
| SEQ ID NO: 132 | CTCCATCAATTACT | SEQ ID NO: 360 | CTGTTCTAGAAGGC |
| SEQ ID NO: 133 | TCTCCATCAATTAC | SEQ ID NO: 361 | ACTGTTCTAGAAGG |
| SEQ ID NO: 134 | TTCTCCATCAATTA | SEQ ID NO: 362 | TACTGTTCTAGAAG |
| SEQ ID NO: 135 | TTTCTCCATCAATT | SEQ ID NO: 363 | CTACTGTTCTAGAA |
| SEQ ID NO: 136 | GTTTCTCCATCAAT | SEQ ID NO: 364 | TCTACTGTTCTAGA |
| SEQ ID NO: 137 | GGTTTCTCCATCAA | SEQ ID NO: 365 | GTCTACTGTTCTAG |
| SEQ ID NO: 138 | AGGTTTCTCCATCA | SEQ ID NO: 366 | TGTCTACTGTTCTA |
| SEQ ID NO: 139 | CAGGTTTCTCCATC | SEQ ID NO: 367 | GTGTCTACTGTTCT |
| SEQ ID NO: 140 | ACAGGTTTCTCCAT | SEQ ID NO: 368 | TGTGTCTACTGTTC |
| SEQ ID NO: 141 | GACAGGTTTCTCCA | SEQ ID NO: 369 | TTGTGTCTACTGTT |
| SEQ ID NO: 142 | AGACAGGTTTCTCC | SEQ ID NO: 370 | TTTGTGTCTACTGT |
| SEQ ID NO: 143 | GAGACAGGTTTCTC | SEQ ID NO: 371 | TTTTGTGTCTACTG |
| SEQ ID NO: 144 | AGAGACAGGTTTCT | SEQ ID NO: 372 | GTTTTGTGTCTACT |
| SEQ ID NO: 145 | AAGAGACAGGTTTC | SEQ ID NO: 373 | TGTTTTGTGTCTAC |
| SEQ ID NO: 146 | CAAGAGACAGGTTT | SEQ ID NO: 374 | CTGTTTTGTGTCTA |
| SEQ ID NO: 147 | CCAAGAGACAGGTT | SEQ ID NO: 375 | CCTGTTTTGTGTCT |
| SEQ ID NO: 148 | TCCAAGAGACAGGT | SEQ ID NO: 376 | GCCTGTTTTGTGTC |
| SEQ ID NO: 149 | ATCCAAGAGACAGG | SEQ ID NO: 377 | AGCCTGTTTTGTGT |
| SEQ ID NO: 150 | TATCCAAGAGACAG | SEQ ID NO: 378 | GAGCCTGTTTTGTG |
| SEQ ID NO: 151 | ATATCCAAGAGACA | SEQ ID NO: 379 | TGAGCCTGTTTTGT |
| SEQ ID NO: 152 | AATATCCAAGAGAC | SEQ ID NO: 380 | CTGAGCCTGTTTTG |
| SEQ ID NO: 153 | GAATATCCAAGAGA | SEQ ID NO: 381 | CCTGAGCCTGTTTT |
| SEQ ID NO: 154 | AGAATATCCAAGAG | SEQ ID NO: 382 | TCCTGAGCCTGTTT |
| SEQ ID NO: 155 | GAGAATATCCAAGA | SEQ ID NO: 383 | GTCCTGAGCCTGTT |
| SEQ ID NO: 156 | CGAGAATATCCAAG | SEQ ID NO: 384 | AGTCCTGAGCCTGT |
| SEQ ID NO: 157 | TCGAGAATATCCAA | SEQ ID NO: 385 | AAGTCCTGAGCCTG |
| SEQ ID NO: 158 | GTCGAGAATATCCA | SEQ ID NO: 386 | TAAGTCCTGAGCCT |
| SEQ ID NO: 159 | TGTCGAGAATATCC | SEQ ID NO: 387 | CTAAGTCCTGAGCC |
| SEQ ID NO: 160 | GTGTCGAGAATATC | SEQ ID NO: 388 | GCTAAGTCCTGAGC |
| SEQ ID NO: 161 | TGTGTCGAGAATAT | SEQ ID NO: 389 | TGCTAAGTCCTGAG |
| SEQ ID NO: 162 | CTGTGTCGAGAATA | SEQ ID NO: 390 | TTGCTAAGTCCTGA |
| SEQ ID NO: 163 | GCTGTGTCGAGAAT | SEQ ID NO: 391 | CTTGCTAAGTCCTG |
| SEQ ID NO: 164 | TGCTGTGTCGAGAA | SEQ ID NO: 392 | TCTTGCTAAGTCCT |
| SEQ ID NO: 165 | CTGCTGTGTCGAGA | SEQ ID NO: 393 | TTCTTGCTAAGTCC |
| SEQ ID NO: 166 | CCTGCTGTGTCGAG | SEQ ID NO: 394 | CTTCTTGCTAAGTC |
| SEQ ID NO: 167 | ACCTGCTGTGTCGA | SEQ ID NO: 395 | ACTTCTTGCTAAGT |
| SEQ ID NO: 168 | GACCTGCTGTGTCG | SEQ ID NO: 396 | AACTTCTTGCTAAG |
| SEQ ID NO: 169 | TGACCTGCTGTGTC | SEQ ID NO: 397 | TAACTTCTTGCTAA |
| SEQ ID NO: 170 | TTGACCTGCTGTGT | SEQ ID NO: 398 | ATAACTTCTTGCTA |
| SEQ ID NO: 171 | CTTGACCTGCTGTG | SEQ ID NO: 399 | CATAACTTCTTGCT |
| SEQ ID NO: 172 | TCTTGACCTGCTGT | SEQ ID NO: 400 | CCATAACTTCTTGC |
| SEQ ID NO: 173 | CTCTTGACCTGCTG | SEQ ID NO: 401 | TCCATAACTTCTTG |
| SEQ ID NO: 174 | CCTCTTGACCTGCT | SEQ ID NO: 402 | TTCCATAACTTCTT |
| SEQ ID NO: 175 | TCCTCTTGACCTGC | SEQ ID NO: 403 | ATTCCATAACTTCT |
| SEQ ID NO: 176 | CTCCTCTTGACCTG | SEQ ID NO: 404 | AATTCCATAACTTC |
| SEQ ID NO: 177 | ACTCCTCTTGACCT | SEQ ID NO: 405 | GAATTCCATAACTT |
| SEQ ID NO: 178 | TACTCCTCTTGACC | SEQ ID NO: 406 | GGAATTCCATAACT |
| SEQ ID NO: 179 | GTACTCCTCTTGAC | SEQ ID NO: 407 | AGGAATTCCATAAC |
| SEQ ID NO: 180 | TGTACTCCTCTTGA | SEQ ID NO: 408 | AAGGAATTCCATAA |
| SEQ ID NO: 181 | CTGTACTCCTCTTG | SEQ ID NO: 409 | AAAGGAATTCCATA |
| SEQ ID NO: 182 | ACTGTACTCCTCTT | SEQ ID NO: 410 | AAAAGGAATTCCAT |
| SEQ ID NO: 183 | CACTGTACTCCTCT | SEQ ID NO: 411 | TAAAAGGAATTCCA |
| SEQ ID NO: 184 | GCACTGTACTCCTC | SEQ ID NO: 412 | ATAAAAGGAATTCC |
| SEQ ID NO: 185 | TGCACTGTACTCCT | SEQ ID NO: 413 | AATAAAAGGAATTC |
| SEQ ID NO: 186 | TTGCACTGTACTCC | SEQ ID NO: 414 | CAATAAAAGGAATT |
| SEQ ID NO: 187 | ATTGCACTGTACTC | SEQ ID NO: 415 | TCAATAAAAGGAAT |
| SEQ ID NO: 188 | CATTGCACTGTACT | SEQ ID NO: 416 | TTCAATAAAAGGAA |
| SEQ ID NO: 189 | TCATTGCACTGTAC | SEQ ID NO: 417 | TTTCAATAAAAGGA |
| SEQ ID NO: 190 | CTCATTGCACTGTA | SEQ ID NO: 418 | GTTTCAATAAAAGG |
| SEQ ID NO: 191 | CCTCATTGCACTGT | SEQ ID NO: 419 | TGTTTCAATAAAAG |
| SEQ ID NO: 192 | CCCTCATTGCACTG | SEQ ID NO: 420 | ATGTTTCAATAAAA |
| SEQ ID NO: 193 | TCCCTCATTGCACT | SEQ ID NO: 421 | GATGTTTCAATAAA |
| SEQ ID NO: 194 | GTCCCTCATTGCAC | SEQ ID NO: 422 | TGATGTTTCAATAA |
| SEQ ID NO: 195 | GGTCCCTCATTGCA | SEQ ID NO: 423 | CTGATGTTTCAATA |
| SEQ ID NO: 196 | TGGTCCCTCATTGC | SEQ ID NO: 424 | GCTGATGTTTCAAT |
| SEQ ID NO: 197 | CTGGTCCCTCATTG | SEQ ID NO: 425 | TGCTGATGTTTCAA |
| SEQ ID NO: 198 | ACTGGTCCCTCATT | SEQ ID NO: 426 | TTGCTGATGTTTCA |
| SEQ ID NO: 199 | TACTGGTCCCTCAT | SEQ ID NO: 427 | TTTGCTGATGTTTC |
| SEQ ID NO: 200 | GTACTGGTCCCTCA | SEQ ID NO: 428 | CTTTGCTGATGTTT |
| SEQ ID NO: 201 | TGTACTGGTCCCTC | SEQ ID NO: 429 | TCTTTGCTGATGTT |
| SEQ ID NO: 202 | ATGTACTGGTCCCT | SEQ ID NO: 430 | GTCTTTGCTGATGT |
| SEQ ID NO: 203 | CATGTACTGGTCCC | SEQ ID NO: 431 | TGTCTTTGCTGATG |
| SEQ ID NO: 204 | TCATGTACTGGTCC | SEQ ID NO: 432 | TTGTCTTTGCTGAT |
| SEQ ID NO: 205 | CTCATGTACTGGTC | SEQ ID NO: 433 | CTTGTCTTTGCTGA |
| SEQ ID NO: 206 | CCTCATGTACTGGT | SEQ ID NO: 434 | TCTTGTCTTTGCTG |
| SEQ ID NO: 207 | TCCTCATGTACTGG | SEQ ID NO: 435 | GTCTTGTCTTTGCT |
| SEQ ID NO: 208 | GTCCTCATGTACTG | SEQ ID NO: 436 | TGTCTTGTCTTTGC |
| SEQ ID NO: 209 | AGTCCTCATGTACT | SEQ ID NO: 437 | CTGTCTTGTCTTTG |
| SEQ ID NO: 210 | CAGTCCTCATGTAC | SEQ ID NO: 438 | TCTGTCTTGTCTTT |
| SEQ ID NO: 211 | CCAGTCCTCATGTA | SEQ ID NO: 439 | CTCTGTCTTGTCTT |
| SEQ ID NO: 212 | CCCAGTCCTCATGT | SEQ ID NO: 440 | TCTCTGTCTTGTCT |
| SEQ ID NO: 213 | CCCCAGTCCTCATG | SEQ ID NO: 441 | CTCTCTGTCTTGTC |
| SEQ ID NO: 214 | TCCCCAGTCCTCAT | SEQ ID NO: 442 | ACTCTCTGTCTTGT |
| SEQ ID NO: 215 | CTCCCCAGTCCTCA | SEQ ID NO: 443 | CACTCTCTGTCTTG |
| SEQ ID NO: 216 | CCTCCCCAGTCCTC | SEQ ID NO: 444 | CCACTCTCTGTCTT |
| SEQ ID NO: 217 | CCCTCCCCAGTCCT | SEQ ID NO: 445 | TCCACTCTCTGTCT |
| SEQ ID NO: 218 | GCCCTCCCCAGTCC | SEQ ID NO: 446 | CTCCACTCTCTGTC |
| SEQ ID NO: 219 | AGCCCTCCCCAGTC | SEQ ID NO: 447 | CCTCCACTCTCTGT |
| SEQ ID NO: 220 | AAGCCCTCCCCAGT | SEQ ID NO: 448 | TCCTCCACTCTCTG |
| SEQ ID NO: 221 | AAAGCCCTCCCCAG | SEQ ID NO: 449 | ATCCTCCACTCTCT |
| SEQ ID NO: 222 | GAAAGCCCTCCCCA | SEQ ID NO: 450 | CATCCTCCACTCTC |
| SEQ ID NO: 223 | AGAAAGCCCTCCCC | SEQ ID NO: 451 | GCATCCTCCACTCT |
| SEQ ID NO: 224 | AAGAAAGCCCTCCC | SEQ ID NO: 452 | AGCATCCTCCACTC |
| SEQ ID NO: 225 | AAAGAAAGCCCTCC | SEQ ID NO: 453 | AAGCATCCTCCACT |
| SEQ ID NO: 226 | CAAAGAAAGCCCTC | SEQ ID NO: 454 | AAAGCATCCTCCAC |
| SEQ ID NO: 227 | ACAAAGAAAGCCCT | SEQ ID NO: 455 | AAAAGCATCCTCCA |
| SEQ ID NO: 228 | CACAAAGAAAGCCC | SEQ ID NO: 456 | AAAAAGCATCCTCC |
| SEQ ID NO: 229 | ACACAAAGAAAGCC | SEQ ID NO: 457 | TAAAAAGCATCCTC |
| SEQ ID NO: 230 | TACACAAAGAAAGC | SEQ ID NO: 458 | ATAAAAAGCATCCT |
| SEQ ID NO: 231 | ATACACAAAGAAAG | SEQ ID NO: 459 | TATAAAAAGCATCC |
| SEQ ID NO: 232 | AATACACAAAGAAA | SEQ ID NO: 460 | GTATAAAAAGCATC |
| SEQ ID NO: 233 | AAATACACAAAGAA | SEQ ID NO: 461 | TGTATAAAAAGCAT |
| SEQ ID NO: 234 | CAAATACACAAAGA | SEQ ID NO: 462 | ATGTATAAAAAGCA |
| SEQ ID NO: 235 | GCAAATACACAAAG | SEQ ID NO: 463 | AATGTATAAAAAGC |
| SEQ ID NO: 236 | GGCAAATACACAAA | SEQ ID NO: 464 | CAATGTATAAAAAG |
| SEQ ID NO: 237 | TGGCAAATACACAA | SEQ ID NO: 465 | CCAATGTATAAAAA |
| SEQ ID NO: 238 | ATGGCAAATACACA | SEQ ID NO: 466 | ACCAATGTATAAAA |
| SEQ ID NO: 239 | TATGGCAAATACAC | SEQ ID NO: 467 | CACCAATGTATAAA |
| SEQ ID NO: 240 | TTATGGCAAATACA | SEQ ID NO: 468 | TCACCAATGTATAA |
| SEQ ID NO: 241 | TTTATGGCAAATAC | SEQ ID NO: 469 | CTCACCAATGTATA |
| SEQ ID NO: 242 | ATTTATGGCAAATA | SEQ ID NO: 470 | TCTCACCAATGTAT |
| SEQ ID NO: 243 | TATTTATGGCAAAT | SEQ ID NO: 471 | CTCTCACCAATGTA |
| SEQ ID NO: 244 | TTATTTATGGCAAA | SEQ ID NO: 472 | TCTCTCACCAATGT |
| SEQ ID NO: 245 | ATTATTTATGGCAA | SEQ ID NO: 473 | CTCTCTCACCAATG |
| SEQ ID NO: 246 | TATTATTTATGGCA | SEQ ID NO: 474 | TCTCTCTCACCAAT |
| SEQ ID NO: 247 | GTATTATTTATGGC | SEQ ID NO: 475 | ATCTCTCTCACCAA |
| SEQ ID NO: 248 | AGTATTATTTATGG | SEQ ID NO: 476 | GATCTCTCTCACCA |
| SEQ ID NO: 249 | TAGTATTATTTATG | SEQ ID NO: 477 | GGATCTCTCTCACC |
| SEQ ID NO: 250 | TTAGTATTATTTAT | SEQ ID NO: 478 | CGGATCTCTCTCAC |
| SEQ ID NO: 251 | TTTAGTATTATTTA | SEQ ID NO: 479 | TCGGATCTCTCTCA |
| SEQ ID NO: 252 | ATTTAGTATTATTT | SEQ ID NO: 480 | GTCGGATCTCTCTC |
| SEQ ID NO: 253 | GATTTAGTATTATT | SEQ ID NO: 481 | TGTCGGATCTCTCT |
| SEQ ID NO: 254 | TGATTTAGTATTAT | SEQ ID NO: 482 | TTGTCGGATCTCTC |
| SEQ ID NO: 255 | ATGATTTAGTATTA | SEQ ID NO: 483 | ATTGTCGGATCTCT |
| SEQ ID NO: 256 | AATGATTTAGTATT | SEQ ID NO: 484 | TATTGTCGGATCTC |
| SEQ ID NO: 257 | AAATGATTTAGTAT | SEQ ID NO: 485 | GTATTGTCGGATCT |
| SEQ ID NO: 258 | CAAATGATTTAGTA | SEQ ID NO: 486 | TGTATTGTCGGATC |
| SEQ ID NO: 259 | TCAAATGATTTAGT | SEQ ID NO: 487 | CTGTATTGTCGGAT |
| SEQ ID NO: 260 | TTCAAATGATTTAG | SEQ ID NO: 488 | TCTGTATTGTCGGA |
| SEQ ID NO: 261 | CTTCAAATGATTTA | SEQ ID NO: 489 | ATCTGTATTGTCGG |
| SEQ ID NO: 262 | TCTTCAAATGATTT | SEQ ID NO: 490 | AATCTGTATTGTCG |
| SEQ ID NO: 263 | ATCTTCAAATGATT | SEQ ID NO: 491 | CAATCTGTATTGTC |
| SEQ ID NO: 264 | TATCTTCAAATGAT | SEQ ID NO: 492 | TCAATCTGTATTGT |
| SEQ ID NO: 265 | ATATCTTCAAATGA | SEQ ID NO: 493 | TTCAATCTGTATTG |
| SEQ ID NO: 266 | AATATCTTCAAATG | SEQ ID NO: 494 | TTTCAATCTGTATT |
| SEQ ID NO: 267 | GAATATCTTCAAAT | SEQ ID NO: 495 | TTTTCAATCTGTAT |
| SEQ ID NO: 268 | TGAATATCTTCAAA | SEQ ID NO: 496 | TTTTTCAATCTGTA |
| SEQ ID NO: 269 | GTGAATATCTTCAA | SEQ ID NO: 497 | TTTTTTCAATCTGT |
| SEQ ID NO: 270 | GGTGAATATCTTCA | SEQ ID NO: 498 | TTTTTTTCAATCTG |
| SEQ ID NO: 271 | TGGTGAATATCTTC | SEQ ID NO: 499 | ATTTTTTTCAATCT |
| SEQ ID NO: 272 | ATGGTGAATATCTT | SEQ ID NO: 500 | GATTTTTTTCAATC |
| SEQ ID NO: 273 | AATGGTGAATATCT | SEQ ID NO: 501 | TGATTTTTTTCAAT |
| SEQ ID NO: 274 | TAATGGTGAATATC | SEQ ID NO: 502 | CTGATTTTTTTCAA |
| SEQ ID NO: 275 | ATAATGGTGAATAT | SEQ ID NO: 503 | GCTGATTTTTTTCA |
| SEQ ID NO: 276 | TATAATGGTGAATA | SEQ ID NO: 504 | TGCTGATTTTTTTC |
| SEQ ID NO: 277 | CTATAATGGTGAAT | SEQ ID NO: 505 | TTGCTGATTTTTTT |
| SEQ ID NO: 278 | TCTATAATGGTGAA | SEQ ID NO: 506 | TTTGCTGATTTTTT |
| SEQ ID NO: 279 | CTCTATAATGGTGA | SEQ ID NO: 507 | CTTTGCTGATTTTT |
| SEQ ID NO: 280 | TCTCTATAATGGTG | SEQ ID NO: 508 | TCTTTGCTGATTTT |
| SEQ ID NO: 281 | TTCTCTATAATGGT | SEQ ID NO: 509 | TTCTTTGCTGATTT |
| SEQ ID NO: 282 | GTTCTCTATAATGG | SEQ ID NO: 510 | CTTCTTTGCTGATT |
| SEQ ID NO: 283 | TGTTCTCTATAATG | SEQ ID NO: 511 | TCTTCTTTGCTGAT |
| SEQ ID NO: 284 | TTGTTCTCTATAAT | SEQ ID NO: 512 | TTCTTCTTTGCTGA |
| SEQ ID NO: 285 | TTTGTTCTCTATAA | SEQ ID NO: 513 | TTTCTTCTTTGCTG |
| SEQ ID NO: 286 | ATTTGTTCTCTATA | SEQ ID NO: 514 | TTTTCTTCTTTGCT |
| SEQ ID NO: 287 | AATTTGTTCTCTAT | SEQ ID NO: 515 | CTTTTCTTCTTTGC |
| SEQ ID NO: 288 | TAATTTGTTCTCTA | SEQ ID NO: 516 | TCTTTTCTTCTTTG |
| SEQ ID NO: 289 | TTAATTTGTTCTCT | SEQ ID NO: 517 | GTCTTTTCTTCTTT |
| SEQ ID NO: 290 | TTTAATTTGTTCTC | SEQ ID NO: 518 | AGTCTTTTCTTCTT |
| SEQ ID NO: 291 | TTTTAATTTGTTCT | SEQ ID NO: 519 | GAGTCTTTTCTTCT |
| SEQ ID NO: 292 | CTTTTAATTTGTTC | SEQ ID NO: 520 | GGAGTCTTTTCTTC |
| SEQ ID NO: 293 | TCTTTTAATTTGTT | SEQ ID NO: 521 | AGGAGTCTTTTCTT |
| SEQ ID NO: 294 | CTCTTTTAATTTGT | SEQ ID NO: 522 | CAGGAGTCTTTTCT |
| SEQ ID NO: 295 | ACTCTTTTAATTTG | SEQ ID NO: 523 | CCAGGAGTCTTTTC |
| SEQ ID NO: 296 | AACTCTTTTAATTT | SEQ ID NO: 524 | GCCAGGAGTCTTTT |
| SEQ ID NO: 297 | TAACTCTTTTAATT | SEQ ID NO: 525 | AGCCAGGAGTCTTT |
| SEQ ID NO: 298 | TTAACTCTTTTAAT | SEQ ID NO: 526 | CAGCCAGGAGTCTT |
| SEQ ID NO: 299 | CTTAACTCTTTTAA | SEQ ID NO: 527 | ACAGCCAGGAGTCT |
| SEQ ID NO: 300 | CCTTAACTCTTTTA | SEQ ID NO: 528 | CACAGCCAGGAGTC |
| SEQ ID NO: 301 | TCCTTAACTCTTTT | SEQ ID NO: 529 | ACACAGCCAGGAGT |
| SEQ ID NO: 302 | GTCCTTAACTCTTT | SEQ ID NO: 530 | CACACAGCCAGGAG |
| SEQ ID NO: 303 | AGTCCTTAACTCTT | SEQ ID NO: 531 | TCACACAGCCAGGA |
| SEQ ID NO: 304 | GAGTCCTTAACTCT | SEQ ID NO: 532 | TTCACACAGCCAGG |
| SEQ ID NO: 305 | AGAGTCCTTAACTC | SEQ ID NO: 533 | TTTCACACAGCCAG |
| SEQ ID NO: 306 | CAGAGTCCTTAACT | SEQ ID NO: 534 | TTTTCACACAGCCA |
| SEQ ID NO: 307 | TCAGAGTCCTTAAC | SEQ ID NO: 535 | ATTTTCACACAGCC |
| SEQ ID NO: 308 | TTCAGAGTCCTTAA | SEQ ID NO: 536 | AATTTTCACACAGC |
| SEQ ID NO: 309 | CTTCAGAGTCCTTA | SEQ ID NO: 537 | TAATTTTCACACAG |
| SEQ ID NO: 310 | TCTTCAGAGTCCTT | SEQ ID NO: 538 | TTAATTTTCACACA |
| SEQ ID NO: 311 | ATCTTCAGAGTCCT | SEQ ID NO: 539 | TTTAATTTTCACAC |
| SEQ ID NO: 312 | CATCTTCAGAGTCC | SEQ ID NO: 540 | TTTTAATTTTCACA |
| SEQ ID NO: 313 | ACATCTTCAGAGTC | SEQ ID NO: 541 | TTTTTAATTTTCAC |
| SEQ ID NO: 314 | TACATCTTCAGAGT | SEQ ID NO: 542 | TTTTTTAATTTTCA |
| SEQ ID NO: 315 | GTACATCTTCAGAG | SEQ ID NO: 543 | ATTTTTTAATTTTC |
| SEQ ID NO: 316 | GGTACATCTTCAGA | SEQ ID NO: 544 | CATTTTTTAATTTT |
| SEQ ID NO: 317 | AGGTACATCTTCAG | SEQ ID NO: 545 | GCATTTTTTAATTT |
| SEQ ID NO: 318 | TAGGTACATCTTCA | SEQ ID NO: 546 | TGCATTTTTTAATT |
| SEQ ID NO: 319 | ATAGGTACATCTTC | SEQ ID NO: 547 | ATGCATTTTTTAAT |
| SEQ ID NO: 320 | CATAGGTACATCTT | SEQ ID NO: 548 | AATGCATTTTTTAA |
| SEQ ID NO: 321 | CCATAGGTACATCT | SEQ ID NO: 549 | TAATGCATTTTTTA |
| SEQ ID NO: 322 | ACCATAGGTACATC | SEQ ID NO: 550 | ATAATGCATTTTTT |
| SEQ ID NO: 323 | GACCATAGGTACAT | SEQ ID NO: 551 | TATAATGCATTTTT |
| SEQ ID NO: 324 | GGACCATAGGTACA | SEQ ID NO: 552 | TTATAATGCATTTT |
| SEQ ID NO: 325 | AGGACCATAGGTAC | SEQ ID NO: 553 | ATTATAATGCATTT |
| SEQ ID NO: 326 | TAGGACCATAGGTA | SEQ ID NO: 554 | CATTATAATGCATT |
| SEQ ID NO: 327 | CTAGGACCATAGGT | SEQ ID NO: 555 | ACATTATAATGCAT |
| | | SEQ ID NO: 556 | TACATTATAATGCA |

TABLE 8
Exemplary selected KRAS antisense oligonucleotide

| | Nucleic acid | | Nucleic acid |
|---|---|---|---|
| SEQ ID NO | sequence | SEQ ID NO | sequence |
| SEQ ID NO: 129 | CATCAATTACTACT | SEQ ID NO: 439 | CTCTGTCTTGTCTT |
| SEQ ID NO: 213 | CCCCAGTCCTCATG | SEQ ID NO: 440 | TCTCTGTCTTGTCT |
| SEQ ID NO: 214 | TCCCCAGTCCTCAT | SEQ ID NO: 441 | CTCTCTGTCTTGTC |
| SEQ ID NO: 215 | CTCCCCAGTCCTCA | SEQ ID NO: 494 | TTTCAATCTGTATT |
| SEQ ID NO: 216 | CCTCCCCAGTCCTC | SEQ ID NO: 495 | TTTTCAATCTGTAT |
| SEQ ID NO: 217 | CCCTCCCCAGTCCT | SEQ ID NO: 496 | TTTTTCAATCTGTA |
| SEQ ID NO: 250 | TTAGTATTATTTAT | SEQ ID NO: 497 | TTTTTTCAATCTGT |
| SEQ ID NO: 251 | TTTAGTATTATTTA | SEQ ID NO: 503 | GCTGATTTTTTTCA |
| SEQ ID NO: 252 | ATTTAGTATTATTT | SEQ ID NO: 504 | TGCTGATTTTTTTC |
| SEQ ID NO: 253 | GATTTAGTATTATT | SEQ ID NO: 505 | TTGCTGATTTTTTT |
| SEQ ID NO: 254 | TGATTTAGTATTAT | SEQ ID NO: 506 | TTTGCTGATTTTTT |
| SEQ ID NO: 255 | ATGATTTAGTATTA | SEQ ID NO: 507 | CTTTGCTGATTTTT |
| SEQ ID NO: 256 | AATGATTTAGTATT | SEQ ID NO: 508 | TCTTTGCTGATTTT |
| SEQ ID NO: 392 | TCTTGCTAAGTCCT | SEQ ID NO: 509 | TTCTTTGCTGATTT |
| SEQ ID NO: 393 | TTCTTGCTAAGTCC | SEQ ID NO: 510 | CTTCTTTGCTGATT |
| SEQ ID NO: 394 | CTTCTTGCTAAGTC | SEQ ID NO: 511 | TCTTCTTTGCTGAT |
| SEQ ID NO: 399 | CATAACTTCTTGCT | SEQ ID NO: 512 | TTCTTCTTTGCTGA |
| SEQ ID NO: 400 | CCATAACTTCTTGC | SEQ ID NO: 513 | TTTCTTCTTTGCTG |
| SEQ ID NO: 401 | TCCATAACTTCTTG | SEQ ID NO: 514 | TTTTCTTCTTTGCT |
| SEQ ID NO: 402 | TTCCATAACTTCTT | SEQ ID NO: 515 | CTTTTCTTCTTTGC |
| SEQ ID NO: 427 | TTTGCTGATGTTTC | SEQ ID NO: 516 | TCTTTTCTTCTTTG |
| SEQ ID NO: 428 | CTTTGCTGATGTTT | SEQ ID NO: 517 | GTCTTTTCTTCTTT |
| SEQ ID NO: 429 | TCTTTGCTGATGTT | SEQ ID NO: 518 | AGTCTTTTCTTCTT |
| SEQ ID NO: 430 | GTCTTTGCTGATGT | SEQ ID NO: 519 | GAGTCTTTTCTTCT |
| SEQ ID NO: 433 | CTTGTCTTTGCTGA | SEQ ID NO: 520 | GGAGTCTTTTCTTC |
| SEQ ID NO: 434 | TCTTGTCTTTGCTG | SEQ ID NO: 521 | AGGAGTCTTTTCTT |
| SEQ ID NO: 435 | GTCTTGTCTTTGCT | SEQ ID NO: 522 | CAGGAGTCTTTTCT |
| SEQ ID NO: 436 | TGTCTTGTCTTTGC | SEQ ID NO: 018 | TATCGTCAAGGCACTC |
| SEQ ID NO: 437 | CTGTCTTGTCTTTG | SEQ ID NO: 019 | CCTCATTGCACTGTAC |
| SEQ ID NO: 438 | TCTGTCTTGTCTTT | SEQ ID NO: 020 | TATCCAAACTGCCCTA |

TABLE 9
Exemplary KRAS antisense oligonucleotide targeting mutated KRAS

| | Nucleic acid | | Nucleic acid |
|---|---|---|---|
| SEQ ID NO | sequence | SEQ ID NO | sequence |
| SEQ ID NO: | TGCCTACGCCACAAGC | SEQ ID NO: | GCCATCAGCTCCAAC |
| 024 | | 042 | T |
| SEQ ID NO: | GCCTACGCCACAAGCT | SEQ ID NO: | CCATCAGCTCCAACT |
| 025 | | 043 | A |
| SEQ ID NO: | CCTACGCCACAAGCTC | SEQ ID NO: | ACGCCACAAGCTCCA |
| 026 | | 065 | |
| SEQ ID NO: | CTACGCCACAAGCTCC | SEQ ID NO: | CGCCACAAGCTCCA |
| 027 | | 066 | |
| SEQ ID NO: | TACGCCACAAGCTCCA | SEQ ID NO: | ACGCCACAAGCTCC |
| 028 | | 067 | |
| SEQ ID NO: | ACGCCACAAGCTCCA | SEQ ID NO: | CTACGCCATCAGCTC |
| 029 | A | 068 | |
| SEQ ID NO: | CGCCACAAGCTCCAA | SEQ ID NO: | CTACGCCATCAGCT |
| 030 | C | 069 | |
| SEQ ID NO: | GCCACAAGCTCCAACT | SEQ ID NO: | TACGCCATCAGCTC |
| 031 | | 070 | |
| SEQ ID NO: | CCACAAGCTCCAACTA | SEQ ID NO: | ACGCCATCAGCTCC |
| 032 | | 071 | |
| SEQ ID NO: | CACAAGCTCCAACTAC | SEQ ID NO: | CCTACGCCAGCAGCT |
| 033 | | 072 | C |
| SEQ ID NO: | TTGCCTACGCCATCAG | SEQ ID NO: | CTACGCCAGCAGCTC |
| 034 | | 073 | |
| SEQ ID NO: | TGCCTACGCCATCAGC | SEQ ID NO: | CTACGCCAGCAGCT |
| 035 | | 074 | |
| SEQ ID NO: | GCCTACGCCATCAGCT | SEQ ID NO: | TACGCCAGCAGCTC |
| 036 | | 075 | |
| SEQ ID NO: | CCTACGCCATCAGCTC | SEQ ID NO: | ACGCCAGCAGCTCC |
| 037 | | 076 | |
| SEQ ID NO: | CTACGCCATCAGCTCC | SEQ ID NO: | CCTACGCCAACAGCT |
| 038 | | 077 | C |
| SEQ ID NO: | TACGCCATCAGCTCCA | SEQ ID NO: | CTACGCCAACAGCTC |
| 039 | | 078 | |
| SEQ ID NO: | ACGCCATCAGCTCCAA | SEQ ID NO: | CTACGCCAACAGCT |
| 040 | | 079 | |
| SEQ ID NO: | CGCCATCAGCTCCAAC | SEQ ID NO: | TACGCCAACAGCTC |
| 041 | | 080 | |
| | | SEQ ID NO: | ACGCCAACAGCTCC |
| | | 081 | |
| SEQ ID NO: | CGCCACAAGCTCCA | SEQ ID NO: | CTACGCCAGCAGCTC |
| 082 | | 087 | C |

TABLE 10
Negative control sequences used in the present
application

| SEQ ID NO | Nucleic acid sequence |
|---|---|
| SEQ ID NO: 1 | CACGTCTATACACCAC |
| SEQ ID NO: 17 | TTTCTGAGGATTATCG |

### EXAMPLES

The following illustrative examples are representative of embodiments of the stimulation, systems, and methods described herein and are not meant to be limiting in any way.

### Example 1. Knockdown of KRAS mRNA

### Cell Culture Condition and In Vitro Transfection

NCI-H358 cell line with KRAS G12C mutation was plated at a density of 20,000 cells per well in a 96-well plates and were treated with both 5 nM and 20 nM of antisense oligonucleotide by transfection with Lipofectamine (Life Technology, USA). The transfection was conducted according to vendor's recommendation, with 0.3 μL Lipofectamine per well and incubated for 3 hours. After 2 days, cells were harvested and subjected to Quantigene assay for relative mRNA quantitation analysis (Life Technology, USA) and followed the specification from vendor. The catalog numbers of KRAS and PPIB (reference gene to normalize the expression) probes are SA-50338 and SA-50155 respectively. The percent reduction of mRNA against a non-targeting control oligonucleotide were calculated and summarized in Table 1.

TABLE 1
Knockdown of KRAS mRNA by antisense oligonucleotide

| Cell | Target KRAS | | % Inhibition | |
|---|---|---|---|---|
| lines | mRNA | Sequence | 20 nM | 5 nM |
| NCI- | Negative | CACGTCTATACACCAC | 0 | 0 |
| H358 | control | (SEQ ID NO: 1) | | |
| NCI- | Negative | TTTCTGAGGATTATCG | −9.3 | −18.2 |
| H358 | control | (SEQ ID NO: 17) | | |
| NCI- | hKRAS | TATCGTCAAGGCACTC | 21.1 | 10.8 |
| H358 | | (SEQ ID NO: 18) | | |
| NCI- | hKRAS | CCTCATTGCACTGTAC | 36.5 | 33.4 |
| H358 | | (SEQ ID NO: 19) | | |
| NCI- | hKRAS | TATCCAAACTGCCCTA | 14.3 | 10.8 |
| H358 | | (SEQ ID NO: 20) | | |

### Example 2. Inhibition of pERK

NCI-H358 cell line with KRAS G12C mutation was plated at a density of 20,000 cells per well in a 96-well plates and were treated with both 5 nM and 20 nM of antisense oligonucleotide by transfection with Lipofectamine (Life Technology, USA). The transfection was conducted according to vendor's recommendation, with 0.3 μL Lipofectamine per well and incubated for 3 hours. After 4 days, cells were harvested and subjected to pERK AlphaLISA assay (Cat. ALSU-PERK-A10K, Perkin Elmer, USA). The pERK inhibition for each treatment was calculated by normalizing with a non-targeting control oligo and summarized in the table below.

TABLE 2
pERK inhibition induced by KRAS knockdown

| Cell | Target KRAS | | % Inhibition | |
|---|---|---|---|---|
| lines | mRNA | Sequence | 20 nM | 5 nM |
| NCI- | Negative | CACGTCTATACACCAC | 0 | 0 |
| H358 | control | (SEQ ID NO: 1) | | |
| NCI- | Negative | TTTCTGAGGATTATCG | 5.1 | 11.3 |
| H358 | control | (SEQ ID NO: 17) | | |
| NCI- | hKRAS | TATCGTCAAGGCACTC | 49 | 34.5 |
| H358 | | (SEQ ID NO: 18) | | |
| NCI- | hKRAS | CCTCATTGCACTGTAC | 65 | 66.6 |
| H358 | | (SEQ ID NO: 19) | | |
| NCI- | hKRAS | TATCCAAACTGCCCTA | 41 | 30.7 |
| H358 | | (SEQ ID NO: 20) | | |
| NCI- | hKRAS G12C | TGCCTACGCCACAAGC | 59.1 | 49.4 |
| H358 | | (SEQ ID NO: 24) | | |
| NCI- | hKRAS G12C | GCCTACGCCACAAGCT | 59.6 | 45.3 |
| H358 | | (SEQ ID NO: 25) | | |
| NCI- | hKRAS G12C | CCTACGCCACAAGCTC | 49.6 | 49.1 |
| H358 | | (SEQ ID NO: 26) | | |
| NCI- | hKRAS G12C | CTACGCCACAAGCTCC | 55.5 | 47.7 |
| H358 | | (SEQ ID NO: 27) | | |
| NCI- | hKRAS G12C | TACGCCACAAGCTCCA | 72.4 | 69.8 |
| H358 | | (SEQ ID NO: 28) | | |
| NCI- | hKRAS G12C | ACGCCACAAGCTCCAA | 53.6 | 52.1 |
| H358 | | (SEQ ID NO: 29) | | |
| NCI- | hKRAS G12C | CGCCACAAGCTCCAAC | 43.1 | 53.6 |
| H358 | | (SEQ ID NO: 30) | | |
| NCI- | hKRAS G12C | GCCACAAGCTCCAACT | 36.9 | 44.1 |
| H358 | | (SEQ ID NO: 31) | | |
| NCI- | hKRAS G12C | CCACAAGCTCCAACTA | 48.3 | 40.7 |
| H358 | | (SEQ ID NO: 32) | | |
| NCI- | hKRAS G12C | CACAAGCTCCAACTAC | 47.3 | 36.4 |
| H358 | | (SEQ ID NO: 33) | | |

### Example 3. Antisense Oligonucleotide Mediated Growth Inhibition of Various Cancer Cells

Various tumor cell lines carrying KRAS mutations (NCI-H358 and LCLC97TM1) and wildtype KRAS (NCI-H1975 and A375) were plated at a density of 800 cells per well in 384 well plates and were treated with both 5 uM and 1 uM of antisense oligonucleotide by coincubation. After 7 days, Cell viability was measured by CellTiter-Glo® 2.0 assay (Promega, USA) according to vendor protocol, and calculated the growth inhibition against a non-targeting control oligo. The results were summarized in the following tables. Pan KRAS ASOs and KRAS mutation matched ASOs displayed growth inhibition in NCI-H358 and LCLC97TM1 cells. KRAS ASOs has limited effect on NCI-H1975 (EGFR mutant) and no or little effect on A375 (BRAF mutant).

TABLE 3
Antisense oligonucleotide mediated growth inhibition in bronchioalveolar
carcinoma

| Cell lines | | | % Inhibition | |
|---|---|---|---|---|
| (G12C) | Target KRAS mRNA | Sequence | 5 μM | 1 μM |
| NCI-H358 | Negative control | CACGTCTATACACCAC | 0 | 0 |
| | | (SEQ ID NO: 1) | | |
| NCI-H358 | hKRAS | GCTATTAGGAGTCTTT | 56.3 | 27.1 |
| | | (SEQ ID NO: 44) | | |
| NCI-H358 | hKRAS G12C | CTACGCCACAAGCTCC | 60.6 | 33 |
| | | (SEQ ID NO: 27) | | |
| NCI-H358 | hKRAS G12C | TACGCCACAAGCTCCA | 65.8 | 46.3 |
| | | (SEQ ID NO: 28) | | |
| NCI-H358 | hKRAS G12D | CCTACGCCATCAGCTC | −14.6 | −8.7 |
| | | (SEQ ID NO: 37) | | |
| NCI-H358 | hKRAS | CCTCATTGCACTGTAC | 56.9 | 32.7 |
| | | (SEQ ID NO: 19) | | |
| NCI-H358 | hKRAS G12C | ACGCCACAAGCTCCA | 63.5 | 38 |
| | | (SEQ ID NO: 65) | | |
| NCI-H358 | hKRAS G12C | CGCCACAAGCTCCA | 55.6 | 39.2 |
| | | (SEQ ID NO: 66) | | |
| NCI-H358 | hKRAS G12C | ACGCCACAAGCTCC | 55.7 | 37.8 |
| | | (SEQ ID NO: 67) | | |
| NCI-H358 | hKRAS G12D | CTACGCCATCAGCTC | −3.8 | −3.5 |
| | | (SEQ ID NO: 68) | | |
| NCI-H358 | hKRAS G12D | CTACGCCATCAGCT | −6.3 | 11.2 |
| | | (SEQ ID NO: 69) | | |
| NCI-H358 | hKRAS G12D | TACGCCATCAGCTC | −16.4 | −1.7 |
| | | (SEQ ID NO: 70) | | |
| NCI-H358 | hKRAS G12D | ACGCCATCAGCTCC | 2.6 | 1.4 |
| | | (SEQ ID NO: 71) | | |
| NCI-H358 | hKRAS G12A | CCTACGCCAGCAGCTC | 5.2 | 1.8 |
| | | (SEQ ID NO: 72) | | |
| NCI-H358 | hKRAS G12A | CTACGCCAGCAGCTC | 8.4 | 3.7 |
| | | (SEQ ID NO: 73) | | |
| NCI-H358 | hKRAS G12A | CTACGCCAGCAGCT | 1 | −7.9 |
| | | (SEQ ID NO: 74) | | |
| NCI-H358 | hKRAS G12A | TACGCCAGCAGCTC | 0.6 | −9.1 |
| | | (SEQ ID NO: 75) | | |
| NCI-H358 | hKRAS G12A | ACGCCAGCAGCTCC | 7.6 | 1.7 |
| | | (SEQ ID NO: 76) | | |
| NCI-H358 | hKRAS G12V | CCTACGCCAACAGCTC | 3.3 | −1.6 |
| | | (SEQ ID NO: 77) | | |
| NCI-H358 | hKRAS G12V | CTACGCCAACAGCTC | −6.2 | −2.5 |
| | | (SEQ ID NO: 78) | | |
| NCI-H358 | hKRAS G12V | CTACGCCAACAGCT | 10.2 | 7.6 |
| | | (SEQ ID NO: 79) | | |
| NCI-H358 | hKRAS G12V | TACGCCAACAGCTC | −15.4 | −15.9 |
| | | (SEQ ID NO: 80) | | |
| NCI-H358 | hKRAS G12V | ACGCCAACAGCTCC | 3.5 | 0.1 |
| | | (SEQ ID NO: 81) | | |

TABLE 4
Antisense oligonucleotide mediated growth inhibition in lung large
cell carcinoma

| Cell lines | | | % Inhibition | |
|---|---|---|---|---|
| (G12V) | Target KRAS mRNA | Sequence | 5 μM | 1 μM |
| LCLC-97TM1 | Negative control | CACGTCTATACACCAC | 0 | 0 |
| | | (SEQ ID NO: 1) | | |
| LCLC-97TM1 | hKRAS | GCTATTAGGAGTCTTT | 90.9 | 84.7 |
| | | (SEQ ID NO: 44) | | |
| LCLC-97TM1 | hKRAS G12C | CTACGCCACAAGCTCC | 21.8 | 7.9 |
| | | (SEQ ID NO: 27) | | |
| LCLC-97TM1 | hKRAS G12C | TACGCCACAAGCTCCA | 45.8 | 41.3 |
| | | (SEQ ID NO: 28) | | |
| LCLC-97TM1 | hKRAS G12D | CCTACGCCATCAGCTC | 57.4 | 17.3 |
| | | (SEQ ID NO: 37) | | |
| LCLC-97TM1 | hKRAS | CCTCATTGCACTGTAC | 88.3 | 77.2 |
| | | (SEQ ID NO: 19) | | |
| LCLC-97TM1 | hKRAS G12C | ACGCCACAAGCTCCA | 29.1 | 19.3 |
| | | (SEQ ID NO: 65) | | |
| LCLC-97TM1 | hKRAS G12C | CGCCACAAGCTCCA | 0.1 | 8.5 |
| | | (SEQ ID NO: 66) | | |
| LCLC-97TM1 | hKRAS G12C | ACGCCACAAGCTCC | 13.9 | 13.6 |
| | | (SEQ ID NO: 67) | | |
| LCLC-97TM1 | hKRAS G12D | CTACGCCATCAGCTC | 22.7 | 20.7 |
| | | (SEQ ID NO: 68) | | |
| LCLC-97TM1 | hKRAS G12D | CTACGCCATCAGCT | −21.5 | 13.4 |
| | | (SEQ ID NO: 69) | | |
| LCLC-97TM1 | hKRAS G12D | TACGCCATCAGCTC | 37.4 | 28.2 |
| | | (SEQ ID NO: 70) | | |
| LCLC-97TM1 | hKRAS G12D | ACGCCATCAGCTCC | 62.3 | 32.7 |
| | | (SEQ ID NO: 71) | | |
| LCLC-97TM1 | hKRAS G12A | CCTACGCCAGCAGCTC | 49.3 | 38.8 |
| | | (SEQ ID NO: 72) | | |
| LCLC-97TM1 | hKRAS G12A | CTACGCCAGCAGCTC | 61.7 | 34.3 |
| | | (SEQ ID NO: 73) | | |
| LCLC-97TM1 | hKRAS G12A | CTACGCCAGCAGCT | 22.3 | 18.3 |
| | | (SEQ ID NO: 74) | | |
| LCLC-97TM1 | hKRAS G12A | TACGCCAGCAGCTC | 50 | 22.7 |
| | | (SEQ ID NO: 75) | | |
| LCLC-97TM1 | hKRAS G12A | ACGCCAGCAGCTCC | 80.5 | 48.8 |
| | | (SEQ ID NO: 76) | | |
| LCLC-97TM1 | hKRAS G12V | CCTACGCCAACAGCTC | 78.7 | 66.9 |
| | | (SEQ ID NO: 77) | | |
| LCLC-97TM1 | hKRAS G12V | CTACGCCAACAGCTC | 84.1 | 70.1 |
| | | (SEQ ID NO: 78) | | |
| LCLC-97TM1 | hKRAS G12V | CTACGCCAACAGCT | 76.8 | 54.2 |
| | | (SEQ ID NO: 79) | | |
| LCLC-97TM1 | hKRAS G12V | TACGCCAACAGCTC | 87.2 | 65.1 |
| | | (SEQ ID NO: 80) | | |
| LCLC-97TM1 | hKRAS G12V | ACGCCAACAGCTCC | 85 | 71.3 |
| | | (SEQ ID NO: 81) | | |

TABLE 5
Antisense oligonucleotide mediated growth inhibition in lung adenocarcinoma cells

| Cell lines | | | % Inhibition | |
|---|---|---|---|---|
| (KRAS WT) | Target KRAS mRNA | Sequence | 5 μM | 1 μM |
| NCI-H1975 | Negative control | CACGTCTATACACCAC | 0 | 0 |
| | | (SEQ ID NO: 1) | | |
| NCI-H1975 | hKRAS | GCTATTAGGAGTCTTT | 12.6 | 10.4 |
| | | (SEQ ID NO: 44) | | |
| NCI-H1975 | hKRAS G12C | CTACGCCACAAGCTCC | 29 | 12.4 |
| | | (SEQ ID NO: 27) | | |
| NCI-H1975 | hKRAS G12C | TACGCCACAAGCTCCA | 32.5 | 12.8 |
| | | (SEQ ID NO: 28) | | |
| NCI-H1975 | hKRAS G12D | CCTACGCCATCAGCTC | −4.8 | −2.4 |
| | | (SEQ ID NO: 37) | | |
| NCI-H1975 | hKRAS | CCTCATTGCACTGTAC | 9.6 | 9 |
| | | (SEQ ID NO: 19) | | |
| NCI-H1975 | hKRAS G12C | ACGCCACAAGCTCCA | 16.8 | 8 |
| | | (SEQ ID NO: 65) | | |
| NCI-H1975 | hKRAS G12C | CGCCACAAGCTCCA | 7.7 | 12 |
| | | (SEQ ID NO: 66) | | |
| NCI-H1975 | hKRAS G12C | ACGCCACAAGCTCC | 10.7 | 17 |
| | | (SEQ ID NO: 67) | | |
| NCI-H1975 | hKRAS G12D | CTACGCCATCAGCTC | −3.1 | 8.1 |
| | | (SEQ ID NO: 68) | | |
| NCI-H1975 | hKRAS G12D | CTACGCCATCAGCT | −6.1 | 5.9 |
| | | (SEQ ID NO: 69) | | |
| NCI-H1975 | hKRAS G12D | TACGCCATCAGCTC | −13.3 | 2.8 |
| | | (SEQ ID NO: 70) | | |
| NCI-H1975 | hKRAS G12D | ACGCCATCAGCTCC | −6.5 | 8.6 |
| | | (SEQ ID NO: 71) | | |
| NCI-H1975 | hKRAS G12A | CCTACGCCAGCAGCTC | 6.3 | 9.1 |
| | | (SEQ ID NO: 72) | | |
| NCI-H1975 | hKRAS G12A | CTACGCCAGCAGCTC | 2.7 | 14.1 |
| | | (SEQ ID NO: 73) | | |
| NCI-H1975 | hKRAS G12A | CTACGCCAGCAGCT | −1.9 | 8.5 |
| | | (SEQ ID NO: 74) | | |
| NCI-H1975 | hKRAS G12A | TACGCCAGCAGCTC | 3 | 13.5 |
| | | (SEQ ID NO: 75) | | |
| NCI-H1975 | hKRAS G12A | ACGCCAGCAGCTCC | 8.1 | 7.9 |
| | | (SEQ ID NO: 76) | | |
| NCI-H1975 | hKRAS G12V | CCTACGCCAACAGCTC | 8.9 | 11.8 |
| | | (SEQ ID NO: 77) | | |
| NCI-H1975 | hKRAS G12V | CTACGCCAACAGCTC | 6.9 | 8.4 |
| | | (SEQ ID NO: 78) | | |
| NCI-H1975 | hKRAS G12V | CTACGCCAACAGCT | −6.6 | 1.7 |
| | | (SEQ ID NO: 79) | | |
| NCI-H1975 | hKRAS G12V | TACGCCAACAGCTC | 10 | 8.8 |
| | | (SEQ ID NO: 80) | | |
| NCI-H1975 | hKRAS G12V | ACGCCAACAGCTCC | 17.8 | 7.1 |
| | | (SEQ ID NO: 81) | | |

TABLE 6
Antisense oligonucleotide mediated growth inhibition in melanoma cells

| Cell lines | | | % Inhibition | |
|---|---|---|---|---|
| (KRAS WT) | Target KRAS mRNA | Sequence | 5 μM | 1 μM |
| A375 | Negative control | CACGTCTATACACCAC | 0 | 0 |
| | | (SEQ ID NO: 1) | | |
| A375 | hKRAS | GCTATTAGGAGTCTTT | −2.2 | −4.6 |
| | | (SEQ ID NO: 44) | | |
| A375 | hKRAS G12C | CTACGCCACAAGCTCC | 3.2 | −2.9 |
| | | (SEQ ID NO: 27) | | |
| A375 | hKRAS G12C | TACGCCACAAGCTCCA | −11.1 | −2.6 |
| | | (SEQ ID NO: 28) | | |
| A375 | hKRAS G12D | CCTACGCCATCAGCTC | −2.7 | −3.2 |
| | | (SEQ ID NO: 37) | | |
| A375 | hKRAS | CCTCATTGCACTGTAC | −5.3 | −13.6 |
| | | (SEQ ID NO: 19) | | |
| A375 | hKRAS G12C | ACGCCACAAGCTCCA | −4 | −10.4 |
| | | (SEQ ID NO: 65) | | |
| A375 | hKRAS G12C | CGCCACAAGCTCCA | 16.8 | 9.9 |
| | | (SEQ ID NO: 66) | | |
| A375 | hKRAS G12C | ACGCCACAAGCTCC | 12.2 | 6.2 |
| | | (SEQ ID NO: 67) | | |
| A375 | hKRAS G12D | CTACGCCATCAGCTC | 11.9 | 0 |
| | | (SEQ ID NO: 68) | | |
| A375 | hKRAS G12D | CTACGCCATCAGCT | 14.6 | 6.5 |
| | | (SEQ ID NO: 69) | | |
| A375 | hKRAS G12D | TACGCCATCAGCTC | 11.8 | 8 |
| | | (SEQ ID NO: 70) | | |
| A375 | hKRAS G12D | ACGCCATCAGCTCC | 7.8 | 6.1 |
| | | (SEQ ID NO: 71) | | |
| A375 | hKRAS G12A | CCTACGCCAGCAGCTC | −7.2 | −3.5 |
| | | (SEQ ID NO: 72) | | |
| A375 | hKRAS G12A | CTACGCCAGCAGCTC | −6.7 | −2.2 |
| | | (SEQ ID NO: 73) | | |
| A375 | hKRAS G12A | CTACGCCAGCAGCT | 6.7 | 0.3 |
| | | (SEQ ID NO: 74) | | |
| A375 | hKRAS G12A | TACGCCAGCAGCTC | 5.7 | 1.7 |
| | | (SEQ ID NO: 75) | | |
| A375 | hKRAS G12A | ACGCCAGCAGCTCC | 11 | 1.5 |
| | | (SEQ ID NO: 76) | | |
| A375 | hKRAS G12V | CCTACGCCAACAGCTC | 4.8 | −3.6 |
| | | (SEQ ID NO: 77) | | |
| A375 | hKRAS G12V | CTACGCCAACAGCTC | 2 | −1.5 |
| | | (SEQ ID NO: 78) | | |
| A375 | hKRAS G12V | CTACGCCAACAGCT | 2.3 | −4.7 |
| | | (SEQ ID NO: 79) | | |
| A375 | hKRAS G12V | TACGCCAACAGCTC | 4 | −3.8 |
| | | (SEQ ID NO: 80) | | |
| A375 | hKRAS G12V | ACGCCAACAGCTCC | 9.8 | 3.4 |
| | | (SEQ ID NO: 81) | | |

### Example 4. KRAS G12C Mutated mRNA and Protein Knockdown, KRAS Pathway Biomarker Modulation and 3D Growth Inhibition by ASO Treatment

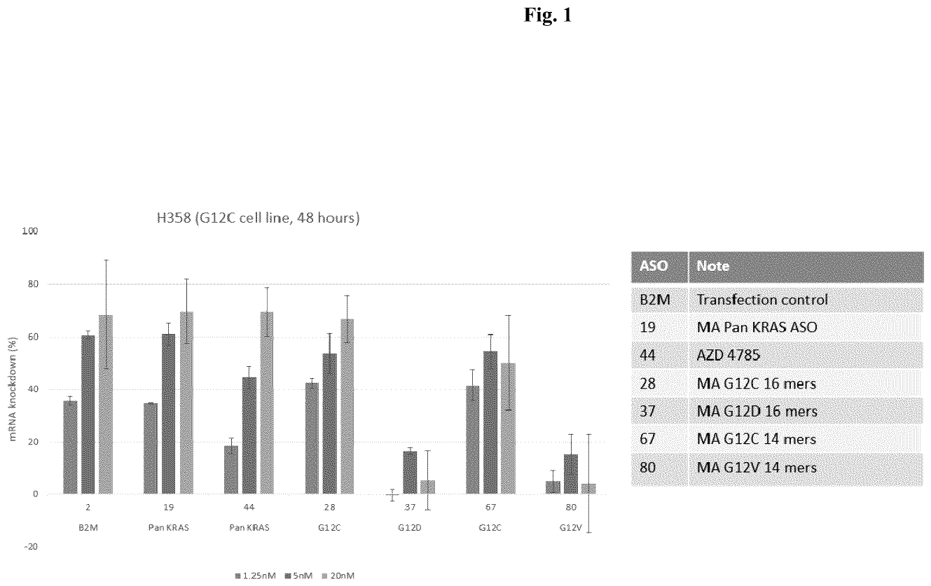

Tumor cells were treated with various ASOs for mRNA knockdown by Quantigene assay, KRAS protein and KRAS pathway downstream biomarkers analysis by Western, and cell growth inhibition by CellTiter-Glo® 2.0 assay. The mRNA knockdown and cell growth inhibition procedures followed the methods described in Example 1 and Example 3, respectively. Western blotting were performed using cells transfected with method described in Example 1, and harvested 3 days post transfection for protein analysis. FIG. 1 illustrates mRNA knockdown of mutated KRAS having the G12C mutation (H358 cell) with ASO SEQ ID NO: 19, 44, 28, 37, 67, or 80. The knockdown of the mRNA of mutated KRAS having the G12C mutation was more pronounced when mediated by ASO SEQ ID NO: 28 and ASO SEQ ID NO: 67, similar to ASO targeting wildtype KRAS (ASO SEQ ID NO: 19 and ASO SEQ ID NO:44). The ASO designed to target KRAS G12D (ASO SEQ ID NO: 12) and targeting KRAS G12V (ASO SEQ ID NO: 80) has no significant mRNA knockdown detected.

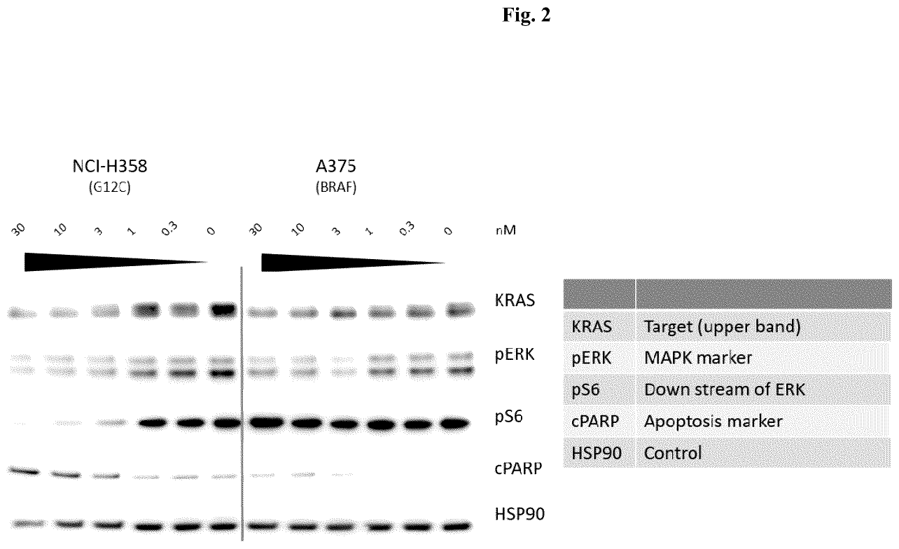

For protein level analysis, cells were seeded in 12 well plate at 10,000 cell/well and transfected with ASO/Lipofectamine (3 ul lipofectamine). Cells were harvested 3 days post transfection for protein expression analysis. The ASO used in the study was ASO SEQ ID NO:28, and the cell lines used were H358 (G12C, heterozygote for KRAS mutation) and A375 (KRAS wild type). FIG. 2 illustrates knockdown of protein expression of both KRAS and protein downstream of the KRAS-RAF-MEK-ERK signaling pathway (pERK, a MAPK marker; and pS6, downstream marker of ERK). FIG. 2 also illustrates increased apoptosis marker (cPARP) expression mediated by increased amount of the ASO SEQ ID NO:28 used for transfection.

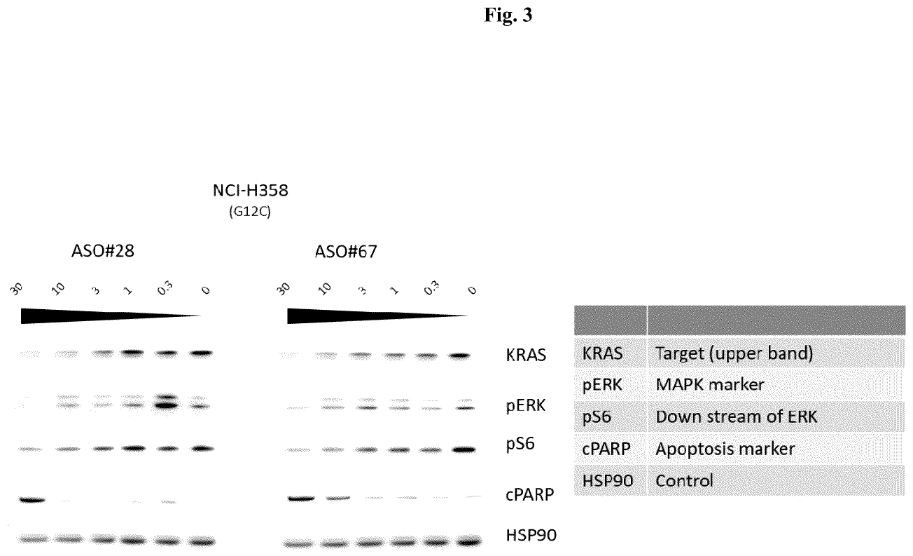

FIG. 3 illustrates another experiment of knockdown of protein expression of both KRAS and protein downstream of the KRAS-RAF-MEK-ERK signaling pathway (pERK, a MAPK marker; and pS6, downstream marker of ERK). Cells were seeded in 6-well plate at 100,000 cell/well and transfected with ASO/Lipofectamine (3 ul lipofectamine). Cells were harvested 3 days post transfection for protein expression analysis. The ASO used in the study was ASO SEQ ID NO:28 (G12C ASO, 16 mers) and ASO SEQ ID NO:67 (G12C ASO, 14 mers). The cell line used was H358 (G12C, heterozygote for KRAS mutation). Protein expression of both KRAS and protein downstream of the KRAS-RAF-MEK-ERK signaling pathway (pERK, a MAPK marker; and pS6, downstream marker of ERK) was decreased in cells transfected with ASO SEQ ID NO:28 or ASO SEQ ID NO:67 in a dose dependent manner. FIG. 3 also illustrates increased apoptosis marker (cPARP) expression mediated by increased amount of the ASO (ASO SEQ ID NO:28 and ASO SEQ ID NO:67) used for transfection.

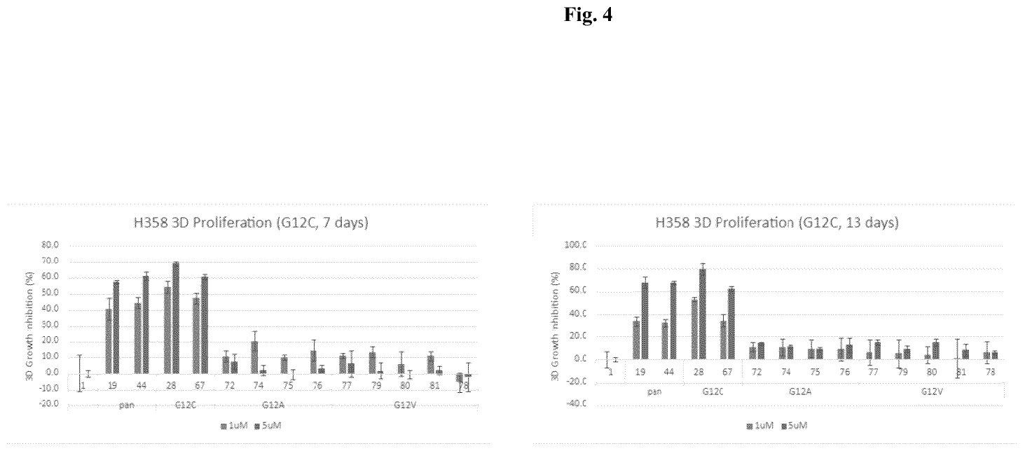

FIG. 4 illustrates 3D cell proliferation inhibition due to inhibiting expression of mutated KRAS by contacting the cell harboring the KRAS mutation (H358 cell having a KRAS G12C mutation) with the oligonucleotide described herein. H358 cell (having G12C KRAS mutation) 3D growth was measured at 7 days (left) and at 13 days (right) after ASO treatment.

### Example 5. KRAS G12V Mutated mRNA and Protein Knockdown, KRAS Pathway Biomarker Modulation and 3D Growth Inhibition by ASO Treatment

All procedure followed methods described in Example 4. The cell lines used were NCI-H441 and LCLC97TM1 (KRAS G12V) and A375 (KRAS wild type).

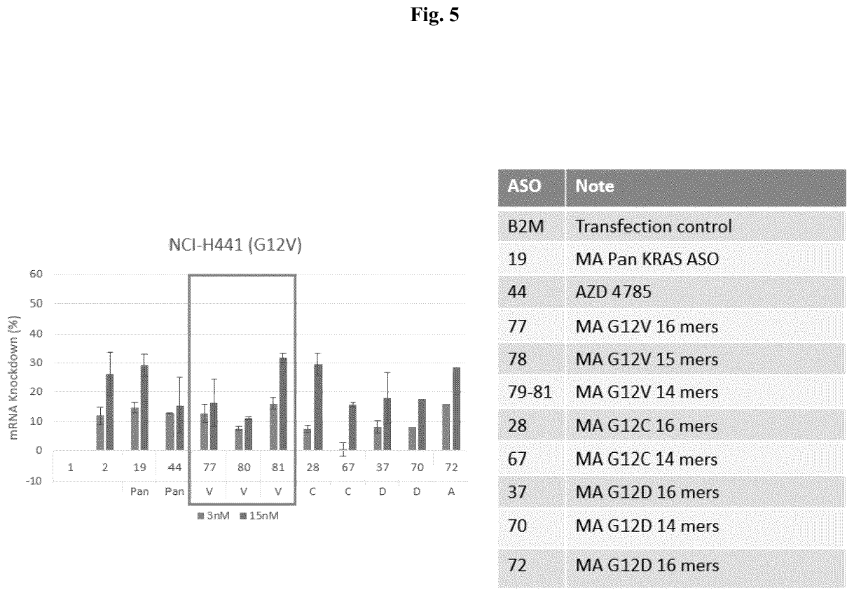

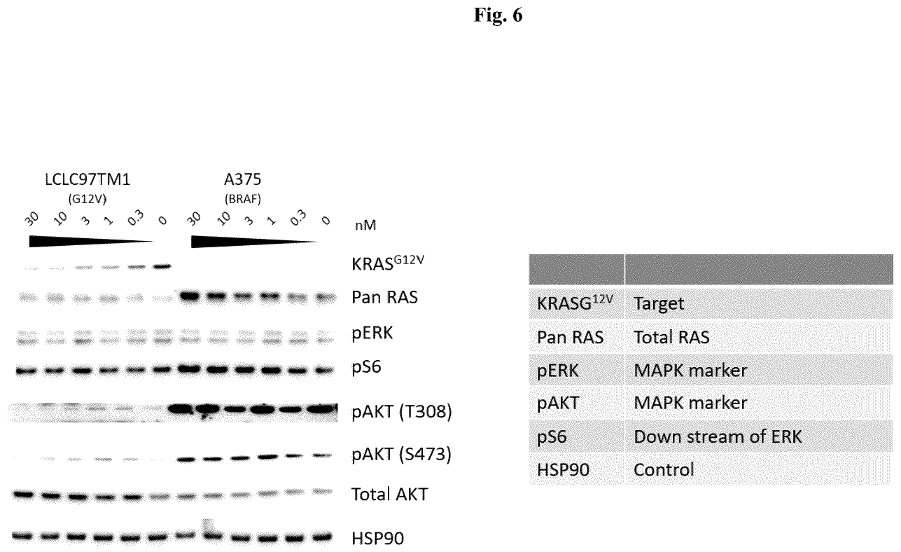

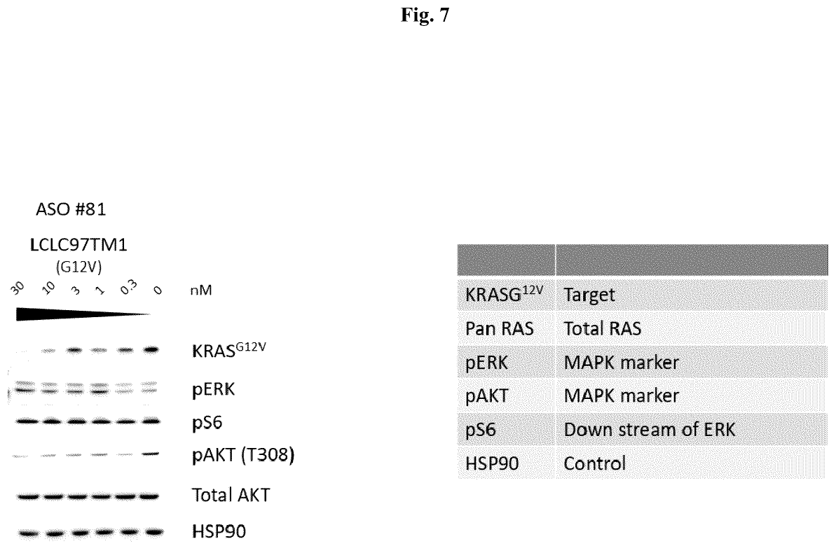

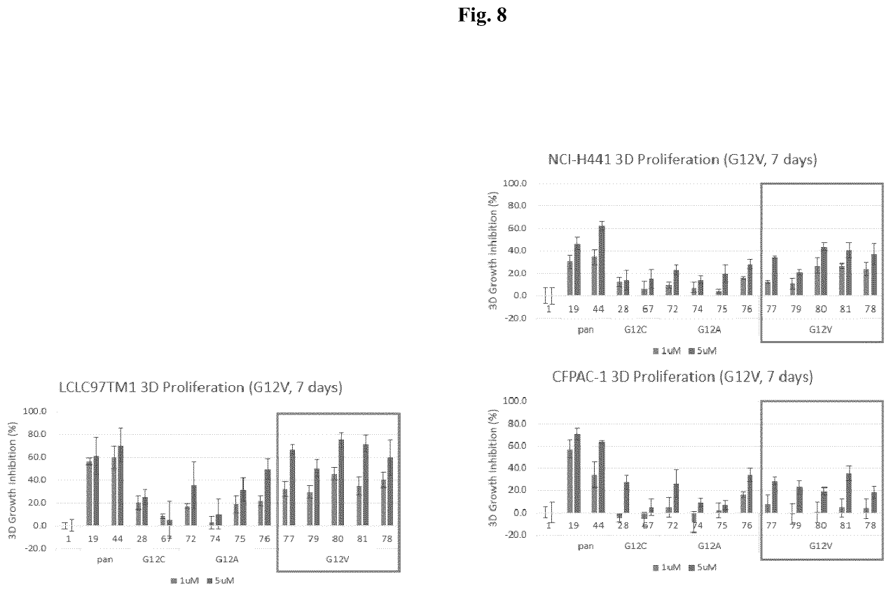

FIG. 5 illustrates mRNA knockdown of mutated KRAS having the G12V mutation (NCI-H441 cell) with ASOs noted herein. FIG. 6 illustrates KRAS protein knockdown (mediated by ASO SEQ ID NO:80) of protein expression of both G12V mutant KRAS and protein downstream of the KRAS-RAF-MEK-ERK signaling pathway (pERK and pAKT, both MAPK marker; and pS6, downstream marker of ERK). FIG. 7 illustrates KRAS protein knockdown (mediated by ASO SEQ ID NO:81) of protein expression of both G12V mutant KRAS and protein downstream of the KRAS-RAF-MEK-ERK signaling pathway (pERK and pAKT, both MAPK marker; and pS6, downstream marker of ERK). FIG. 8 illustrates 3D cell proliferation inhibition due to inhibiting expression of mutated KRAS by contacting the cell harboring the KRAS mutation (LCLC97TM1 cell, NCI-H441 cell, or CFPAC-1 cell) with the oligonucleotide described herein.

### Example 6. KRAS G12A Tumor Cell Growth Inhibition by ASO Treatment

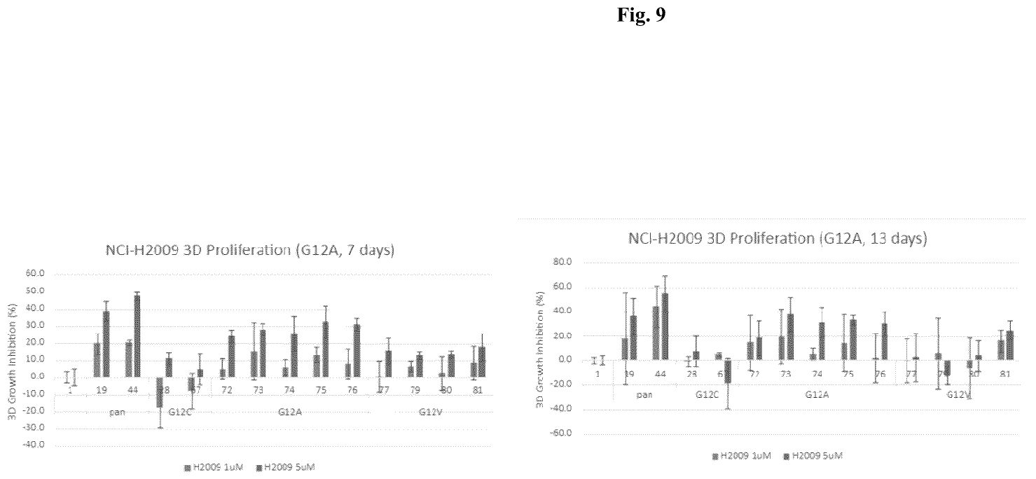

For cell growth inhibition, cells were treated as described in Example 3. NCI-H2009 was used as KRAS G12A mutated tumor cell line. The results are illustrated in FIG. 9. FIG. 9 illustrates 3D cell proliferation inhibition due to inhibiting expression of mutated KRAS by contacting the cell harboring the KRAS mutation (NCI-H2009 cell) with the oligonucleotide described herein. 3D growth was measured at 7 days (left) or 13 days (right) after ASO treatment.

### Example 7. Mutated KRAS (G12D Mutation) mRNA Knockdown by ASO Treatment

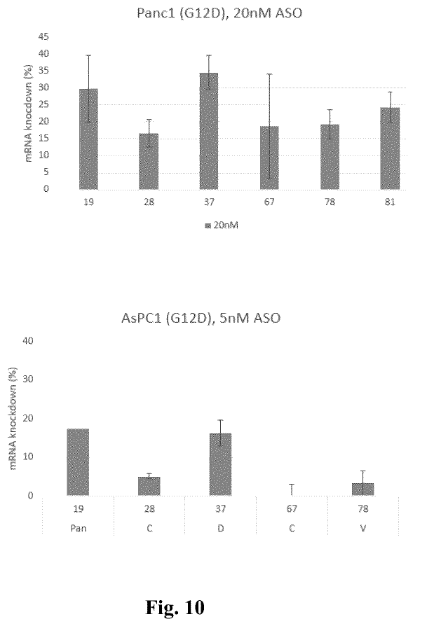

Follow the procedure in Example 1, KRAS G12D tumor cells were treated with ASO and detected for KRAS mRNA by Quantigene assay 48 hours post transfection. FIG. 10 illustrates mRNA knockdown of mutated KRAS having the G12D mutation Panc1 and AsPC1 cells with ASOs noted herein.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

1. A composition comprising an antisense oligonucleotide capable of binding to KRAS mRNA.

2. The composition of claim 1, wherein the KRAS mRNA is a mutated KRAS mRNA.

3. The composition of claim 1, wherein the antisense oligonucleotide comprises a sequence that is at least 80%, 85%, or 90% identical to one of the following sequences: SEQ ID NOs: 100-556.

4. The composition of claim 1, wherein the antisense oligonucleotide comprises a nucleic acid sequence that is at least 80%, 85%, or 90% identical to any one of the following sequences: SEQ ID NOs: 24-43, 65-82, or 87.

5. The composition of claim 1, wherein the antisense oligonucleotide comprises a sequence that is at least 80%, 85%, or 90% identical to any one of the following sequences
SEQ ID NO: 129, SEQ ID NO: 213, SEQ ID NO: 214, SEQ ID NO: 215, SEQ ID NO: 216, SEQ ID NO: 217, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 392, SEQ ID NO: 393, SEQ ID NO: 394, SEQ ID NO: 399, SEQ ID NO: 400, SEQ ID NO: 401, SEQ ID NO: 402, SEQ ID NO: 427, SEQ ID NO: 428, SEQ ID NO: 429, SEQ ID NO: 430, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 494, SEQ ID NO: 495, SEQ ID NO: 496, SEQ ID NO: 497, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 18, SEQ ID NO: 19, or SEQ ID NO: 20.

6. The composition of claim 1, wherein the antisense oligonucleotide comprises a nucleic acid sequence that is at least 80%, 85%, or 90% identical to any one of SEQ ID NO: 18-20, SEQ ID NOs: 24-43, SEQ ID NO: 65-82, or SEQ ID NO: 87.

7. The composition of claim 1, wherein the antisense oligonucleotide comprises 12-30 nucleotides in length.

8. The composition of claim 1, wherein the antisense oligonucleotide comprises a gap segment and a wing segment.

9. The composition of claim 8, wherein the antisense oligonucleotide comprises a 5′-wing segment and a 3′-wing segment.

10. (canceled)

11. The composition of claim 1, wherein the antisense oligonucleotide comprises at least one 2′-modified nucleoside, at least one modified internucleotide linkage, or at least one inverted abasic moiety.

12. The composition of claim 11, wherein the at least one 2′ modified nucleotide: comprises 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleotide, locked nucleic acid (LNA), constrained ethyl (cEt) sugar, ethylene nucleic acid (ENA), or a combination thereof.

13. The composition of claim 11, wherein the at least one modified internucleotide linkage comprises a phosphorothioate linkage or a phosphorodithioate linkage.

14. The composition of claim 1, wherein the antisense oligonucleotide comprises a phosphorodiamidate morpholino oligomer (PMO), locked nucleic acid (LNA), a thiomorpholino, constrained ethyl (cEt) sugar, or a combination thereof.

15. The composition of claim 1, wherein the antisense oligonucleotide is conjugated with a peptide, antibody, lipid, carbohydrates, aptamer or a polymer.

16.-21. (canceled)

22. The composition of claim 1, wherein the antisense oligonucleotide comprises a nucleic acid sequence that is at least 80%, 85%, or 90% identical to any one of SEQ ID NOs: 19, 27, 28, 37, 44, or 65-81.

23. The composition of claim 22, wherein the antisense oligonucleotide comprises a nucleic acid sequence that is at least 80%, 85%, or 90% identical to any one of SEQ ID NOs: 19, 28, 44, 67, 72-77, or 79-81.

24. The composition of claim 1, wherein the antisense oligonucleotide comprises a nucleic acid sequence that is any one of SEQ ID NOs: 19, 27, 28, 37, 44, or 65-81.

25. A method of modulating KRAS-mediated signaling pathway in a cancer cell, comprising: treating the cancer cell with a composition comprising antisense oligonucleotide capable of binding to KRAS mRNA or mutated KRAS mRNA, thereby reducing expression of KRAS mRNA or mutated KRAS mRNA in the cancer cell.

26. (canceled)

27. The method of claim 25, wherein the antisense oligonucleotide comprises a sequence having at least 80%, 85%, or 90% similarity to one of the following sequences: SEQ ID NOs: 100-556.

28. The method of claim 25, wherein the antisense oligonucleotide comprises a sequence having at least 80%, 85%, or 90% similarity to one of the following sequences: SEQ ID NOs: 24-43, 65-82, or 87.

29. The method of claim 25, wherein the antisense oligonucleotide comprises a sequence having at least 80%, 85%, or 90% similarity to one of the following sequences:
SEQ ID NO: 129, SEQ ID NO: 213, SEQ ID NO: 214, SEQ ID NO: 215, SEQ ID NO: 216, SEQ ID NO: 217, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 392, SEQ ID NO: 393, SEQ ID NO: 394, SEQ ID NO: 399, SEQ ID NO: 400, SEQ ID NO: 401, SEQ ID NO: 402, SEQ ID NO: 427, SEQ ID NO: 428, SEQ ID NO: 429, SEQ ID NO: 430, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 494, SEQ ID NO: 495, SEQ ID NO: 496, SEQ ID NO: 497, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 18, SEQ ID NO: 19, or SEQ ID NO: 20.

30.-33. (canceled)

34. The method of claim 25, wherein the expression of KRAS protein, mutated KRAS protein, KRAS mRNA, or mutated KRAS mRNA is decreased by at least 30%, at least 40%, at least 50% after the treatment.

35.-45. (canceled)
